SEQUENCE LISTING (103 sequences)

SEQ ID NO: 1 - DNA, 20 bp, Artificial sequence
  Synthetic primer
cccatcacagaccagcacat

SEQ ID NO: 2 - DNA, 20 bp, Artificial sequence
  Synthetic primer
ctcgtaggccagcttcacaa

SEQ ID NO: 3 - DNA, 18 bp, Artificial sequence
  Synthetic primer
ccccctgacgacccttta

SEQ ID NO: 4 - DNA, 22 bp, Artificial sequence
  Synthetic primer
ggcagtggtttcttacgaagga

SEQ ID NO: 5 - DNA, 24 bp, Artificial sequence
  Synthetic primer
agtgatggcatggactgtggtcat

SEQ ID NO: 6 - DNA, 24 bp, Artificial sequence
  Synthetic primer
catgttcgtcatgggtgtgaacca

SEQ ID NO: 7 - DNA, 20 bp, Artificial sequence
  Synthetic primer
acccgtttggctccaccctt

SEQ ID NO: 8 - DNA, 20 bp, Artificial sequence
  Synthetic primer
aatggggtgaggccggtgct

SEQ ID NO: 9 - DNA, 20 bp, Artificial sequence
  Synthetic primer
ggagactctcagggtcgaaa

SEQ ID NO: 10 - DNA, 20 bp, Artificial sequence
  Synthetic primer
ggattagggcttcctcttgg

SEQ ID NO: 11 - DNA, 21 bp, Artificial sequence
  Synthetic primer
ccctcggtgtcctacttcaaa

SEQ ID NO: 12 - DNA, 21 bp, Artificial sequence
  Synthetic primer
cacctcctcctcctcctcttc

SEQ ID NO: 13 - DNA, 23 bp, Artificial sequence
  Synthetic primer
ccaggagttacttctatgcctga

SEQ ID NO: 14 - DNA, 21 bp, Artificial sequence
  Synthetic primer
ttcatccaggggaggtacaac

SEQ ID NO: 15 - DNA, 22 bp, Artificial sequence
  Synthetic primer
atggctacctctcgatatgagc

SEQ ID NO: 16 - DNA, 21 bp, Artificial sequence
  Synthetic primer
cattggggactctcacactct

SEQ ID NO: 17 - DNA, 21 bp, Artificial sequence
  Synthetic primer
ccagatggctctaacctcagt

SEQ ID NO: 18 - DNA, 22 bp, Artificial sequence
  Synthetic primer
aacttccacgaaaaagaggctt

SEQ ID NO: 19 - DNA, 21 bp, Artificial sequence
  Synthetic primer
gagctggtggttgactttctc

SEQ ID NO: 20 - DNA, 21 bp, Artificial sequence
  Synthetic primer
tccatctccgattcagtccct

SEQ ID NO: 21 - DNA, 21 bp, Artificial sequence
  Synthetic primer
tacaggctggctcaggactat

SEQ ID NO: 22 - DNA, 22 bp, Artificial sequence
  Synthetic primer
cgcaacattttgtagcactctg

SEQ ID NO: 23 - DNA, 23 bp, Artificial sequence
  Synthetic primer
tttccgtggctcttattcaaact

SEQ ID NO: 24 - DNA, 23 bp, Artificial sequence
  Synthetic primer
gcacagtggtaggaacttctcat

SEQ ID NO: 25 - DNA, 22 bp, Artificial sequence
  Synthetic primer
ccattgagcggaagattcaact

SEQ ID NO: 26 - DNA, 20 bp, Artificial sequence
  Synthetic primer
ctgctggtcccgatatgagg

SEQ ID NO: 27 - DNA, 21 bp, Artificial sequence
  Synthetic primer
aacctgcctacacccctatac

SEQ ID NO: 28 - DNA, 21 bp, Artificial sequence
  Synthetic primer
caccacagcaatatggagagg

SEQ ID NO: 29 - DNA, 21 bp, Artificial sequence
  Synthetic primer
tgaaggcaaagtgacctgtga

SEQ ID NO: 30 - DNA, 20 bp, Artificial sequence
  Synthetic primer
aggcgtccagataggagagc

SEQ ID NO: 31 - DNA, 21 bp, Artificial sequence
  Synthetic primer
tctggttcttacgtctgttgc

SEQ ID NO: 32 - DNA, 21 bp, Artificial sequence
  Synthetic primer
ctgtgcagtccctagctttcc

SEQ ID NO: 33 - DNA, 19 bp, Artificial sequence
  Synthetic primer
agggagggctgacccagat

SEQ ID NO: 34 - DNA, 20 bp, Artificial sequence
  Synthetic primer
ggcggaaaacccaaaacttc

SEQ ID NO: 35 - DNA, 22 bp, Artificial sequence
  Synthetic primer
gcccttggtgtaaattacctgc

SEQ ID NO: 36 - DNA, 19 bp, Artificial sequence
  Synthetic primer
ggacatcatgcgccacttt

SEQ ID NO: 37 - DNA, 20 bp, Artificial sequence
  Synthetic primer
gcaggaaaacgtggatgatt

SEQ ID NO: 38 - DNA, 20 bp, Artificial sequence
  Synthetic primer
catttcttcacaaccgcaaa

SEQ ID NO: 39 - DNA, 24 bp, Artificial sequence
  Synthetic primer
atttatcggagttgcagttgcgcc

SEQ ID NO: 40 - DNA, 24 bp, Artificial sequence
  Synthetic primer
acaaacactacggtaggctgcgaa

SEQ ID NO: 41 - DNA, 21 bp, Artificial sequence
  Synthetic primer
gcatactgagaccctgccttt

SEQ ID NO: 42 - DNA, 26 bp, Artificial sequence
  Synthetic primer
ctcacagaattctttataaaaacacc

SEQ ID NO: 43 - DNA, 21 bp, Artificial sequence
  Synthetic primer
cgggctctctgctgtacatat

SEQ ID NO: 44 - DNA, 20 bp, Artificial sequence
  Synthetic primer
agcatcaggccgtccaatct

SEQ ID NO: 45 - DNA, 30 bp, Artificial sequence
  Synthetic primer
ttctagaccagacactgaccatccactcta

SEQ ID NO: 46 - DNA, 31 bp, Artificial sequence
  Synthetic primer
ggccggccttcactgagtaaacacagtttat

SEQ ID NO: 47 - DNA, 32 bp, Artificial sequence
  Synthetic primer
ttctagagaagtttgaacctaaatctggctgg

SEQ ID NO: 48 - DNA, 37 bp, Artificial sequence
  Synthetic primer
ggccggcctatagagaaaaatacatacaatttattca

SEQ ID NO: 49 - DNA, 26 bp, Artificial sequence
  Synthetic primer
ttctagagaagaaccaaaacatcgtc

SEQ ID NO: 50 - DNA, 30 bp, Artificial sequence
  Synthetic primer
ggccggcctaagagtaatttttgttgcttt

SEQ ID NO: 51 - DNA, 29 bp, Artificial sequence
  Synthetic primer
ttctagatgccagaggaggggcactgggt

SEQ ID NO: 52 - DNA, 32 bp, Artificial sequence
  Synthetic primer
ggccggccgaggctcaaaaactcatctttatt

SEQ ID NO: 53 - DNA, 29 bp, Artificial sequence
  Synthetic primer
ttctagagggggatgggggggcagatctt

SEQ ID NO: 54 - DNA, 32 bp, Artificial sequence
  Synthetic primer
ggccggcctatggtacaaaaatagtttattac

SEQ ID NO: 55 - DNA, 30 bp, Artificial sequence; identical to SEQ ID NO: 45
  Synthetic primer

SEQ ID NO: 56 - DNA, 34 bp, Artificial sequence
  Synthetic primer
aaatacagacacgcttaacaaaaatagtatatca

SEQ ID NO: 57 - DNA, 26 bp, Artificial sequence
  Synthetic primer
ttctagagaagaaccaaaacatcgtc

SEQ ID NO: 58 - DNA, 27 bp, Artificial sequence
  Synthetic primer
ccttcgatgtataggacactgatcaaa

SEQ ID NO: 59 - DNA, 32 bp, Artificial sequence
  Synthetic primer
atgtgtgaggagacattggcttgcagtgcgcg

SEQ ID NO: 60 - DNA, 32 bp, Artificial sequence
  Synthetic primer
atgtgtgaggagtcaatggcttgcagtgcgcg

SEQ ID NO: 61 - DNA, 29 bp, Artificial sequence
  Synthetic primer
tgtgcatatattcggaatgacattttagg

SEQ ID NO: 62 - DNA, 38 bp, Artificial sequence
  Synthetic primer
tgtgcatatattcggaattgaattttagggacatggtg

SEQ ID NO: 63 - DNA, 22 bp, Artificial sequence
  Synthetic primer
ugcauauauguugaaugacauu

SEQ ID NO: 64 - DNA, 22 bp, Artificial sequence
  Synthetic primer
ugcauauauucggaaugacauu

SEQ ID NO: 65 - DNA, 19 bp, Artificial sequence
  Synthetic primer
ugaggagcugcuggcuugc

SEQ ID NO: 66 - DNA, 19 bp, Artificial sequence
  Synthetic primer
ugaggagacauuggcuugc

SEQ ID NO: 67 - DNA, 76 bp, Homo sapiens
taagcgtgtctgtatttatgtgtgaggagctgctggcttgcagtgcgcgtgcacgtggagagctggtgcccggaga

SEQ ID NO: 68 - DNA, 76 bp, Pan troglodytes
taagcgtgtctgtatttatgtgtgaggagctgctggcttgcagtgcgcgtgcacatggagagctggtgcccggaga

SEQ ID NO: 69 - DNA, 76 bp, Macaca mulatta; identical to SEQ ID NO: 67

SEQ ID NO: 70 - DNA, 76 bp, Equus caballus
taagcgtgtctgtatttatgtgtgaggagctgctggcttgctgtgcgtgtgcacgtggagagctggtgcccggaga

SEQ ID NO: 71 - DNA, 76 bp, Bos taurus
taagcgtgtctgtatttatgtgtgaggagctgctggcttgcagtgcgtgtgcacgtggagagctggtgcccggaga

SEQ ID NO: 72 - DNA, 76 bp, Canis lupus; identical to SEQ ID NO: 70

SEQ ID NO: 73 - DNA, 76 bp, Mesocricetus auratus
taagcgtgtctgtatttatgtgtgaggagctgctggcttgctgtgcgcgtgcacgtggagagctggtgcccagaga

SEQ ID NO: 74 - DNA, 76 bp, Mus musculus
taagcgtgtctgtatttatgtgtgaggagctgctggcttgctgtgcgcgtgcccgtggagagctggtgcccagaga

SEQ ID NO: 75 - DNA, 76 bp, Rattus norvegicus
taagcgtgtctgtatttatgtatgaggagctgctggcttgctgtgcgcgtgcccgtggagagctggtgcccagaga

SEQ ID NO: 76 - DNA, 76 bp, Artificial sequence
  Synthetic consensus
  misc_feature (22)..(22): n is a, c, g, t or u
  misc_feature (42)..(42): n is a, c, g, t or u
  misc_feature (48)..(48): n is a, c, g, t or u
  misc_feature (53)..(53): n is a, c, g, t or u
  misc_feature (55)..(55): n is a, c, g, t or u
  misc_feature (72)..(72): n is a, c, g, t or u
taagcgtgtctgtatttatgtntgaggagctgctggcttgcngtgcgngtgcncntggagagctggtgcccngaga

SEQ ID NO: 77 - DNA, 77 bp, Homo sapiens
cgaaggtgtgcatatatgttgaatgacattttagggacatggtgtttttataaagaattctgtgagaaaaaatttaa

SEQ ID NO: 78 - DNA, 77 bp, Pan troglodytes
caaaggtgtgcatatatgttgaatgacattttagggacatggtgtttttataaagaattctgtgagaaaaaatttaa

SEQ ID NO: 79 - DNA, 77 bp, Macaca fascicularis
tgaaggtgtgcatatatgttgaatgacattttagggacatggtatttttataaagaattctgtgagaaaaaatttaa

SEQ ID NO: 80 - DNA, 77 bp, Gorilla gorilla
cgaaggtgtgcatatatgttgaaggacattttagggacatggtgtttttataaagaattctgtgagaaaaaatttaa

SEQ ID NO: 81 - DNA, 75 bp, Mus musculus
agaaattcctgttatttattggatgacattttagggatatgaaatttttataaagaatttgtgagaaaaagttaa

SEQ ID NO: 82 - DNA, 75 bp, Rattus norvegicus
agaaattcccgttatttattggatgacattttagggatatggaacttttataaagaattcgtgaggaaaaattaa

SEQ ID NO: 83 - DNA, 77 bp, Artificial sequence
  Synthetic consensus
  misc_feature (1)..(2): n is a, c, g, t or u
  misc_feature (5)..(6): n is a, c, g, t or u
  misc_feature (8)..(12): n is a, c, g, t or u
  misc_feature (16)..(16): n is a, c, g, t or u
  misc_feature (18)..(18): n is a, c, g, t or u
  misc_feature (22)..(22): n is a, c, g, t or u
  misc_feature (24)..(24): n is a, c, g, t or u
  misc_feature (38)..(38): n is a, c, g, t or u
  misc_feature (42)..(44): n is a, c, g, t or u
  misc_feature (60)..(61): n is a, c, g, t or u
  misc_feature (67)..(67): n is a, c, g, t or u
  misc_feature (72)..(72): n is a, c, g, t or u
  misc_feature (75)..(75): n is a, c, g, t or u
nnaanntnnnnntatntnttgnangacattttaggganatgnnntttttataaagaattnngtgagnaaaanttnaa

SEQ ID NO: 84 - DNA, 63 bp, Homo sapiens
atgatgtaacttgaccttccagagttatggaaattttgtccccatgtaatgaataaattgtat

SEQ ID NO: 85 - DNA, 62 bp, Pan troglodytes
agatgtaacttgaccttccagagttatggaaattttgtccccatgtaatgaataaattgtat

SEQ ID NO: 86 - DNA, 60 bp, Mus musculus
tgaaacttgtctttccagagttgcagatattttgtttcaactttaatgaataaattacta

SEQ ID NO: 87 - DNA, 64 bp, Rattus norvegicus
acaatgaaacttgactttccagagttgcagatattttgtttctactttaatgaataaactacta

SEQ ID NO: 88 - DNA, 62 bp, Equus caballus
atgatgtaacgacattccagagttaccgaaattgtgtccccattttaatgaataaattgcat

SEQ ID NO: 89 - DNA, 64 bp, Artificial sequence
  Synthetic consensus
  misc_feature (1)..(4): n is a, c, g, t or u
  misc_feature (7)..(7): n is a, c, g, t or u
  misc_feature (11)..(12): n is a, c, g, t or u
  misc_feature (14)..(14): n is a, c, g, t or u
  misc_feature (16)..(16): n is a, c, g, t or u
  misc_feature (27)..(29): n is a, c, g, t or u
  misc_feature (32)..(32): n is a, c, g, t or u
  misc_feature (36)..(36): n is a, c, g, t or u
  misc_feature (40)..(41): n is a, c, g, t or u
  misc_feature (43)..(43): n is a, c, g, t or u
  misc_feature (45)..(46): n is a, c, g, t or u
  misc_feature (48)..(48): n is a, c, g, t or u
  misc_feature (59)..(59): n is a, c, g, t or u
  misc_feature (61)..(64): n is a, c, g, t or u
nnnntgnaacnngncnttccagagttnnnganattntgtnncnanntnaatgaataaantnnnn

SEQ ID NO: 90 - DNA, 62 bp, Homo sapiens
gggtttccccttgagcccattttacagatgaggaaactgagtccggagaggaaaagggacat

SEQ ID NO: 91 - DNA, 62 bp, Pan troglodytes; identical to SEQ ID NO: 90

SEQ ID NO: 92 - DNA, 61 bp, Canis lupus
cggtttcccctgagcccattttacaaatgagaaaaccgagtccggagaggaaaaggggctt

SEQ ID NO: 93 - DNA, 60 bp, Bos taurus
cggtttctcctgagctcctttacaaatgagaaaactgagtctggagaggaaagggggctt

SEQ ID NO: 94 - DNA, 56 bp, Mus musculus
ccggttctctttgagcccattttacagaatgctgagtccgaagaggaaaaggggct

SEQ ID NO: 95 - DNA, 56 bp, Rattus norvegicus
cctgttctctttgagcccattaaacagaatgcggagtccggagaggaaaaggggct

SEQ ID NO: 96 - DNA, 62 bp, Artificial sequence
  Synthetic consensus
  misc_feature (1)..(4): n is a, c, g, t or u
  misc_feature (8)..(8): n is a, c, g, t or u
  misc_feature (10)..(10): n is a, c, g, t or u
  misc_feature (12)..(12): n is a, c, g, t or u
  misc_feature (17)..(17): n is a, c, g, t or u
  misc_feature (19)..(20): n is a, c, g, t or u
  misc_feature (22)..(23): n is a, c, g, t or u
  misc_feature (27)..(31): n is a, c, g, t or u
  misc_feature (33)..(33): n is a, c, g, t or u
  misc_feature (35)..(36): n is a, c, g, t or u
  misc_feature (38)..(38): n is a, c, g, t or u
  misc_feature (44)..(44): n is a, c, g, t or u
  misc_feature (46)..(46): n is a, c, g, t or u
  misc_feature (55)..(55): n is a, c, g, t or u
  misc_feature (59)..(59): n is a, c, g, t or u
  misc_feature (61)..(62): n is a, c, g, t or u
nnnnttcncntngagcncnntnnacannnnngnanncngagtcngnagaggaaangggncnn

SEQ ID NO: 97 - DNA, 54 bp, Homo sapiens
gggggcagatcttggactcatgaggaggggcccccctgccctgtggggtcaacc

SEQ ID NO: 98 - DNA, 54 bp, Pan troglodytes; identical to SEQ ID NO: 97

SEQ ID NO: 99 - DNA, 53 bp, Macaca mulatta
gggggcagatcttggactcatgaggaggggccccctgccctgtggggtcaacc

SEQ ID NO: 100 - DNA, 52 bp, Mus musculus
gggggcagacccggactcatgaggaggggcctccctgcctgtggggaccact

SEQ ID NO: 101 - DNA, 53 bp, Rattus norvegicus
gggagcagacccggactcatgaggaagggcttccctgccctgtggagaccact

SEQ ID NO: 102 - DNA, 54 bp, Artificial sequence
  Synthetic consensus
  misc_feature (4)..(4): n is a, c, g, t or u
  misc_feature (10)..(10): n is a, c, g, t or u
  misc_feature (12)..(13): n is a, c, g, t or u
  misc_feature (27)..(27): n is a, c, g, t or u
  misc_feature (32)..(33): n is a, c, g, t or u
  misc_feature (36)..(36): n is a, c, g, t or u
  misc_feature (41)..(41): n is a, c, g, t or u
  misc_feature (47)..(47): n is a, c, g, t or u
  misc_feature (49)..(49): n is a, c, g, t or u
  misc_feature (51)..(51): n is a, c, g, t or u
  misc_feature (54)..(54): n is a, c, g, t or u
gggngcagancnnggactcatgaggangggcnnccntgccntgtggngncnacn

SEQ ID NO: 103 - protein, 599 aa, Homo sapiens
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

(19) United States
(12) Patent Application Publication
(10) Pub. No.: US 2018/0112198 A1
(43) Pub. Date: Apr. 26, 2018

(54) TUMOR SUPPRESSION BY MCPIP1

(71) Applicant: Saint Louis University, St. Louis, MO (US)

(72) Inventors: Jianguo LIU, Fenton, MO (US); Huan NING, Maplewood, MO (US); Wenbao LU, St. Louis, MO (US)

(73) Assignee: Saint Louis University, St. Louis, MO (US)

(21) Appl. No.: 15/566,626

(22) PCT Filed: Mar. 30, 2016
(86) PCT No.: PCT/US16/24829

§ 371 Date: Oct. 13, 2017

Related U.S. Application Data
(60) Provisional application No. 62/148,080, filed on Apr. 15, 2015.

(51) Int. Cl. C12N 9/16; A61K 48/00; A61K 45/06; A61K 38/46; A61P 35/00; C12Q 1/44; C12Q 1/6886

(52) U.S. Cl. CPC C12N 9/16; A61K 48/0058; A61K 45/06; C12Q 2600/158; A61P 35/00; C12Q 1/44; C12Q 1/6886; A61K 38/465

(57) ABSTRACT

The present disclosure relates to the identification of MCPIP1 as tumor suppressor. Methods of employing MCPIP1 to treat cancer are described.

### PRIORITY CLAIM

This application claims benefit of priority to U.S. Provisional Application Ser. No. 62/148,080, filed Apr. 15, 2015, the entire contents of which are hereby incorporated by reference.

### GOVERNMENT SUPPORT CLAUSE

This invention was made with government support under CA163808 awarded by the National Cancer Institute. The government has certain rights in invention.

### BACKGROUND

### I. Field

The present disclosure relates to the fields of oncology, genetics and molecular biology. More particular the disclosure relates to the use of of MCPIP1 as a tumor suppressive cancer therapy.

### II. Related Art

A hallmark of cancer is that cancer cells can evade apoptosis and proliferate uncontrollably, as well as many diseases (Hanahan and Weinberg, 2011, Lowe and Lin, 2000, Steller, 1995 and Thompson, 1995). Apoptosis plays an important role in resistance to conventional therapeutic regimens (Igney and Krammer, 2002 and Kang and Reynolds, 2009). Though the mechanisms of apoptosis are complex and involve many pathways, the ratio of pro-apoptotic to anti-apoptotic genes determines whether cancer cells undergo apoptosis or survival (Kumar, 2000). Most tumor cells evade apoptosis by either increasing the expression of anti-apoptotic genes or decreasing the expression of pro-apoptotic genes. Overexpression of anti-apoptotic proteins in the BCL2 family is associated with a poor cancer prognosis (Yip and J. C. Reed, 2008 and Placzek et al., 2010). Current efforts are ongoing to interfere with BCL2 and its fellow pro-survival family members to help restore the sensitivity of cancer cells to pro-apoptotic signals. While recent progress has broadened the understanding of the apoptosis-evasion mechanisms by cancer cells, how apoptosis resistance develops in cancer cells through posttranscriptional mechanisms remains unknown.

The inventors have previously shown that overexpression of the chemokine MCPIP1 sensitizes mouse macrophages for apoptosis in response to stress signals (Qi et al., 2011). Treatment of HeLa and HepG2 cells with proteasome inhibitor MG-132 reduces cell viability along with MCPIP1 expression (Skalniak et al., 2013). In human neuroblastoma cells, MCPIP1 overexpression decreases cell viability and proliferation (Skalniak et al., 2014). MCPIP1 also stabilizes RGS2 protein through its deubiquitinase activity to suppress breast cancer cell growth (Lyu et al., 2014). However, a role for MCPIP1 in regulation of cancer proliferation remains unclear.

### SUMMARY

Thus, in accordance with the present disclosure, there is provided an isolated polynucleotide encoding MCPIP1, wherein said isolated polynucleotide is operably connected to a heterologous promoter active in a mammalian cancer cell. The heterologous promoter may operable in human cancer cell, or a non-human mammal cancer cell. The promoter may be selected from the group consisting of hsp68, SV40, CMV IE, MKC, GAL4_{UAS}, HSV and β-actin. The promoter may be a tissue specific promoter or an inducible promoter. The promoter may be active in a human breast cancer cell or a human melanoma cell. The polynucleotide may be contained in a replicable expression vector, such as a viral vector, for example selected from the group consisting of a retroviral vector, an adenoviral vector, and adeno-associated viral vector, a vaccinia viral vector, and a herpesviral vector.

Also provided is a method for suppressing the growth, proliferation, migration or metastasis of a cancer cell comprising contacting said cells with an expression cassette comprising a polynucleotide encoding MCPIP1, wherein said polynucleotide is under the control of a promoter operable in eukaryotic cells. The promoter may be heterologous to the polynucleotide sequences. The heterologous promoter may operable in a human cancer cell, or a non-human mammal cancer cell. The promoter may be selected from the group consisting of hsp68, SV40, CMV IE, MKC, GAL4_{UAS}, HSV and β-actin. The promoter may be a tissue specific promoter or an inducible promoter. The promoter may be active in a human breast cancer cell or a human melanoma cell. The expression cassette may further comprise a polyadenylation signal. The polynucleotide may be contained in a replicable expression vector, such as a viral vector, for example selected from the group consisting of a retroviral vector, an adenoviral vector, and adeno-associated viral vector, a vaccinia viral vector, and a herpesviral vector. The replicable expression vector is a non-viral vector, such as one encapsulated in a lipid delivery vehicle or a nanoparticle.

The method may further comprise contacting said cancer cell with a second anti-cancer therapy, such as radiation therapy, gene therapy, hormonal therapy, immunotherapy, toxin therapy or surgery. The cancer cell may be derived from a tissue selected from the group consisting of brain, lung, liver, spleen, kidney, lymph node, small intestine, blood, pancreas, colon, rectal, stomach, breast, endometrium, prostate, testicle, ovary, skin (e.g., melanoma), head and neck, esophagus, bone marrow and blood tissue cancers (leukemia, lymphoma) and multiple myeloma. The cancer cell may be located in a human subject, and the expression cassette may be administered systemically, administered local or regional to a tumor site, administered by intra-tumoral injection or to a resected tumor bed, and/or administered more than once. The method may further comprising assessing MCPIP1 structure, expression and/or function in a sample from said subject.

In still another embodiment, there is provided a method for suppressing the growth, proliferation, migration or metastasis of a cancer cell comprising the step of contacting a cancer cell with MCPIP1 polypeptide. The MCPIP1 polypeptide may be encapsulated in a lipid delivery vehicle or a nanoparticle. The cancer cell may be derived from a tissue selected from the group consisting of brain, lung, liver, spleen, kidney, lymph node, small intestine, blood, pancreas, colon, rectal, stomach, breast, endometrium, prostate, testicle, ovary, skin (e.g., melanoma), head and neck, esophagus, bone marrow and blood tissue cancers (leukemia, lymphoma) and multiple myeloma. The cancer cell may be located in a human subject, and the MCPIP1 polypeptide may be administered systemically, administered local or regional to a tumor site, administered by intra-tumoral injection or to a resected tumor bed, and/or administered more than once. The method may further comprising assessing MCPIP1 structure, expression and/or function in a sample from said subject. The method may further comprise contacting said cancer cell with a second anti-cancer therapy, such as radiation therapy, gene therapy, hormonal therapy, immunotherapy, toxin therapy or surgery.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions and/or rearrangements may be made within the scope of the disclosure without departing from the spirit thereof, and the disclosure includes all such substitutions, modifications, additions and/or rearrangements, including methods, compositions and combinations of such.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein:

FIGS. 1A-I. MCPIP1 expression is impaired in breast tumors and breast tumor cell lines. (FIG. 1A) MCPIP1 expression was measured by qRT-PCR in human breast tumor specimens (n=9) compared to surrounding ‘normal’ breast tissue (n=3; p<0.0001). See also FIGS. 8A-C. (FIG. 1B) MCPIP1 mRNA levels were measured in triple negative breast tumors (n=5) and compared to triple-positive breast tumors (n=14; p=0.0254). (FIG. 1C) Immunofluorescence staining of MCPIP1 with anti-MCPIP1 antibody (red color) in human breast tumor tissues and normal mammary gland tissues. See also FIGS. 8A-C. (FIGS. 1D-E) MCPIP1 protein (FIG. 1D) and mRNA (FIG. 1E) levels were measured respectively by western blot and qRT-PCR in human breast tumor cell lines (MDA-MB-231 and MDA-MB-453) and human normal mammary gland epithelial cell lines (MCF-10A and MCF-12A). (FIG. 1F-G) MCPIP1 protein (FIG. 1F) and mRNA (FIG. 1G) expression were measured respectively by western blot and qRT-PCR in mouse breast tumor cell lines (4T1 and Ts/A) and mouse normal mammary gland epithelial cell lines (Fsk4 and CommD). (FIGS. 1H-I) Immunoblotting analysis of MCPIP1 protein with anti-MCPIP1 antibody with whole cell lysates extracted from mouse breast tumor cell lines with different metastatic potential (FIG. 1H) and their characteristics (FIG. 1I).

FIGS. 2A-F. MCPIP1 induces apoptosis of breast tumor cells. (FIG. 2A) MDA-MB-231 or 4T1 breast tumor cells were generated to express GFP/MCPIP1 fusion protein with a Tet-on inducible system. GFP/MCPIP1 fusion protein was measured by Immunoblotting with anti-GFP antibody after addition of different amounts of Doxycycline. See also FIG. 9A. (FIG. 2B) Caspase 3 and its cleaved products were measured by Immunoblotting with anti-caspase3 antibody at different times after adding 500 ng/ml of Dox to the above two Tet-On cells. See also FIG. 9D. (FIG. 2C) Poly (ADP-ribose) polymerase (PARP)-1 and its cleaved product were measured by Immunoblotting with anti-PARP1 antibody after addition of different amounts of Dox to the above cells. (FIG. 2D) Apoptosis was measured by flow cytometry in the above cells at 48 hours after adding different amounts of Dox. (FIGS. 2E-F) TUNEL staining was performed in MDA-MB-231/Tet-On cells at 48 hours after adding 500 ng/ml of Dox (Dox+). The ratio of TUNEL-positive cells were calculated and plotted on the histogram (FIG. 2F). See also FIG. 9E.

FIGS. 3A-K MCPIP1 selectively inhibits the mRNA expression of anti-apoptotic gene. (FIG. 3A) Clustergram of the apoptosis related genes affected differentially in MDA-MB-231/Tet-On cells at 32 hours after adding Dox (500 ng/ml). MCPIP1: cells with Dox; Control: cells without Dox. See also Table S1. (FIGS. 3B-E) MDA-MB-231/Tet-On cells were induced to express MCPIP1 by Dox (500 ng/ml) for different times. Then total RNA was collected to detect mRNA levels of selected anti-apoptotic genes by qRT-PCR, including bcl2l1 (FIG. 3B), bric3 (FIG. 3C), bcl3 (FIG. 3D), and relb (FIG. 3E). See also FIGS. 10A-B. (FIGS. 3F-I) The mRNA levels of pro-apoptotic genes in the above cells were measured by qRT-PCR, including bad (FIG. 3F), ripk2 (FIG. 3G), fas (FIG. 3H), and dedd2 (FIG. 3I). (FIGS. 3J-K) MDA-MB-231/Tet-On cells were induced to express GFP/MCPIP1 fusion protein by Dox (500 ng/ml) for 36 h, followed by lysate extraction, immunoprecipitation with anti-GFP antibody or IgG, and RNA extraction. RT-PCR was performed to detect anti-apoptotic gene transcripts (FIG. 3J) and pro-apoptotic gene transcripts (FIG. 3K). IL-6 transcript was used as positive control and GAPDH served as negative control.

FIGS. 4A-J. MCPIP1 destabilizes the mRNAs of anti-apoptotic genes via the PIN domain. (FIGS. 4A-D) MDA-MB-231/Tet-On cells were treated with 500 ng/ml of Dox for 36 hours to induce MCPIP1 expression and then ActD and DRB added for different times. The remained anti-apoptotic gene transcripts, bcl2l1 (FIG. 4A), bcl3 (FIG. 4B), birc3 (FIG. 4C), and relb (FIG. 4D), were measured by qRT-PCR. See also FIGS. 11A-G. (FIG. 4E) HEK293 cells were co-transfected with luciferase reporter constructs containing full-length of 3′UTRs of the anti-apoptotic genes and either MCPIP1 or control vector. Luciferase activities were measured in cell lysates after 36 h. β-actin 3′UTR was used as negative control. Results shown represent the mean±SD of four independent experiments (*: p<0.05). (FIG. 4F) Schematic representation of the RNase and zinc finger domains in MCPIP1, and their mutant, D141N and ΔZF. (FIG. 4G) HEK293 cells were co-transfected with luciferase reporters containing 3′UTRs of the anti-apoptotic genes and WT MCPIP1 as well as the two mutants. After 36 h, luciferase activities were measured in cell lysates and compared to empty vector transfected cells. (FIG. 4H) MDA-MB-231 cells were transiently transfected with MCPIP1 and two mutants, respectively. 36 h later, total RNA was extracted to measure the mRNA expression of different genes as indicated by qRT-PCR. (FIG. 4I) MDA-MB-231 cells were transiently transfected with MCPIP1 and two mutants. 48 h later, floating dead cells were counted in each group. n=3 (FIG. 4J) MDA-MB-231 cells were transiently transfected with MCPIP1 and two mutants. 48 h later, whole cell lysates were collected to measure total PARP1 and cleaved PARP1 by western blot.

FIGS. 5A-H. Knocking-down MCPIP1 enhances anti-apoptotic gene transcript stability and increases cell proliferation. (FIG. 5A) Cell lystaes were isolated from the MDA-MB-231 cells infected with scramble-shRNA/lentivirus and three MCPIP1-shRNAs/lentivirus, followed by western blot analysis with MCPIP1 and β-actin antibodies. (FIG. 5B) Total numbers of parental MDA-MB-231 cells and cells infected with scramble/lentivirus or MCPIP1-shRNA#3/lentivirus were counted at the indicated time (n=3). ***: p<0.0001. (FIG. 5C) MTT was performed in the above cells to measure cell proliferation (n=3). ***: p<0.0001. (FIG. 5D) Anti-apoptotic gene mRNAs were measured in the parental MDA-MB-231 cells and cells infected with scramble/lentivirus or MCPIP1-shRNA#3/lentivirus by qRT-PCR. *: p<0.05. (FIGS. 5E-H) MDA-MB-231 cells were infected with scramble/lentivirus or MCPIP1-shRNA#3/lentivirus and then treated with ActD and DRB, followed by collecting RNA at the indicated times to detect the remaining mRNAs of anti-apoptotic genes by qRT-PCR.

FIGS. 6A-J. MCPIP1 binds to stem-loot structure in the 3′UTR of anti-apoptotic genes for mRNA degradation. (FIG. 6A) Schematic representation of the luciferase reporter constructs of bcl2l1 and birc3 containing truncated 3′UTRs without the stem-loop (Δstem-loop). See also FIGS. 12A-E. (FIG. 6B) HEK293 cells were co-transfected with luciferase reporters containing full-length 3′UTRs or truncated 3′UTRs (Δstem-loop) of bcl2l1 and birc3 in the presence of MCPIP1 or control vector. 48 hour later, luciferase activity was measured in cell lysates and shown as relative levels compared to cells transfected with empty vector. β-actin 3′UTR was used as negative control. Results shown represent the mean±SD of four independent experiments. *: p<0.05. **: p<0.01 between two groups. (FIG. 6C) The predicted stem-loop structures of Bcl2l1 (upper) and Birc3 (lower) in their 3′UTRs and mutation strategy (asterisks indicate base substitution). Mutant1 becomes unable to form stem-loop structure (middle) and Mutant2 still forms a stem-loop structure (left). (FIG. 6D) HEK293 cells were co-transfected with luciferase reporters containing full-length 3′UTRs or mutant 3′UTRs of bcl2l1 and birc3 in the presence of either MCPIP1 or control vector. Luciferase activity was measured in cell lysates and shown as relative levels compared to cells transfected with empty vector. Results shown represent the mean±SD of four independent experiments (p<0.05). (FIG. 6E) RNA-EMSA was performed with biotin-labeled probes corresponding to the BCL2L1 3′UTR, including WT probe, mutant probe and linearized probe, in the presence of whole cell lysates (WCL) extracted from MDA-MB-231/Tet-on cells after adding Dox. (FIG. 6F) WCL of MCPIP1-expressed MDA-MB-231/Tet-on cells were incubated with 1 μg of anti-MCPIP1 and anti-GFP antibody and IgG for 30 min, followed by performing RNA-EMSA assay as described above. (FIGS. 6G-H) RNA-EMSA and supershift EMSA were respectively performed with biotin-labeled probes corresponding to the BIRC3 3′UTR, including WT probe, mutant probe and linearized probe, in the presence of whole cell lysates (WCL) extracted from MDA-MB-231/Tet-on cells after adding Dox. (FIGS. 6I-J) RNA-ChIP was performed with genome fragments from MDA-MB-231/Tet-On cells 24 h after GFP/MCPIP1 induction. The pull-down 3′UTR sequences were amplified by PCR with primers flanking the putative stem-loop sequences.

FIGS. 7A-I. MCPIP1 suppresses breast tumor growth and metastasis and inversely correlate with survival of breast cancer patients. (FIG. 7A) Tumor growth curves in NSG mice received 2×10⁶ MDA-MB-231/Tet-On cells (n=8/group). The day of mice fed with Dox-containing water (1 μg/ml) to induce MCPIP1 was indicated with arrow. The x-axis represents days after tumor cell injection. The y-axis represents tumor volume (mm₃=L×W×H; V, volume; W, width; H, high). See also FIGS. 13B, 14B, and 15A. (FIG. 7B) Quantification of the number of nodules on lungs of each mouse 12 days after fed with Dox water. See also FIGS. 13C and 15B. (FIG. 7C) H&E staining of lung sections of tumor-bearing mice fed with Dox water or normal water. Lung tumor colonization was shown with dot lines and the metastatic lesions enlarged in upper-right box. Scale bar, 200 μm and 50 μm, respectively. (FIG. 7D) Tumor growth curves in NSG mice received parental MDA-MB-231 and MDA-MB-231/Tet-On cells (n=3/group). Dox-water was provided at the same time as tumor cell inoculation (day 0). The x-axis represents days after tumor injection. The y-axis represents tumor volume (mm₃=L×W×H; V, volume; W, width; H, high). (FIG. 7E) Total protein was extracted from tumors of mice 4 days after fed with dox-water and used to detect exogenous GFP/MCPIP1 expression by immunoblotting with anti-MCPIP1 antibody. (FIGS. 7F-G) TUNEL staining on tumor tissues of mice 4 days after fed with dox-water or normal water (FIG. 7F). Quantification of the percentage of TUNEL-positive cells per slide (FIG. 7G). (FIG. 7H) Kaplan-Meier survival curve of breast tumor patients with low and high tumor MCPIP1 transcripts (n=251; p=0.025). (FIG. 71) All patients were grouped into MCPIP1 lower (n=164) and higher (n=72) groups based on the levels of MCPIP1 transcript in tumors (p<0.0001).

FIGS. 8A-C. MCPIP1 expression is reduced in breast tumors and tumor cells. (FIG. 8A) MCPIP1 expression was measured by real-time PCR in tumor specimens of nude mice received MDA-MB-231 cells compared to normal mammary gland tissues of the same mouse (n=5). Immunofluorescence staining of MCPIP1 protein with anti-MCPIP1 antibody in MCF-12A and MCF-7 cells (FIG. 8B), and CommD and 4T1 cells (FIG. 8C). Bars: 50 μm (FIG. 8B) and 10 μm (FIG. 8C).

FIGS. 9A-E. MCPIP1 induces tumor cell apoptosis. (FIG. 9A) Exogenous GFP/MCPIP1 fusion protein and endogenous MCPIP1 expression were detected by immunoblotting with anti-MCPIP1 antibody in MDA-MB-231/Tet-On cells (upper panel) and 4T1/Tet-On cells (lower panel). Cell numbers were counted at different times after MCPIP1 induction in MDA-MB-231/Tet-On (FIG. 9B) and 4T1/Tet-On cells (FIG. 9C). (FIG. 9D) Detection of apoptosis-induced protein caspase-3 by immunoblotting in 4T1 and MDA-MB-231 cells at 48 h after transient transfection with Flag-tagged MCPIP1 and its control vector. (FIG. 9E) TUNEL staining of apoptotic 4T1/Tet-On breast tumor cells at 48 h after adding Dox (500 ng/ml).

FIGS. 10A-B. MCPIP1 inhibits BCL2A1 expression. The expression levels of BCL2A1 (FIG. 10A) and IL-6 (FIG. 10B) mRNA were analyzed by real-time PCR in MDA-MB-231/Tet-on cells at different times after adding Dox (500 ng/ml).

FIGS. 11A-G. MCPIP1 does not affect mRNA stability of pro-apoptotic genes. The half-life of BCL2A1 transcript (FIG. 11A) and the half-lives of pro-apoptotic gene transcripts, including Dedd2 (FIG. 11B), Bad (FIG. 11C), Fas (FIG. 11D), Lta (FIG. 11E), and Ripk2 (FIG. 11F), were measured by real-time PCR in MDA-MB-231/Tet-On cells after adding Dox for 36 h. (FIG. 11G) Schematic of 3′UTRs of bcl2l1 and birc3 (upper panel). The results of RNA-IP with HEK293 cells (lower panel). The 3′UTRs reporter plasmids of birc3 and bcl2l1 were transfected into HEK293 cells, followed by immunoprecipitation with anti-GFP antibody or IgG and RNA extraction. Then RT-PCR was performed to detect luciferase mRNA.

FIGS. 12A-E. Putative stem-loon structures in the 3′UTRs of anti-apoptotic genes. The 3′UTR sequences of anti-apoptotic genes, including bcl2l1 (FIG. 12A), birc3 (FIG. 12B), bcl2a1 (FIG. 12C), relb (FIG. 12D), and bcl3 (FIG. 12E), were obtained from different species as indicated with accession number and aligned. The stem-loop sequences (indicated by green box) located in the 3′UTRs of indicated genes were evolutionally conserved and can fold a secondary stem-loop structure (right). Some AU-rich elements (ARE) are also indicated by red box. For conservation analyses of Bcl2L1, Bcl2A1, RELB, BIRC3 and Bcl3 stem-loop structure, the 3′UTR sequences from different species were extracted from the National Center for Biotechnology Information (NCBI) database: Bcl2L1 3′UTR: human (Homo sapiens; accession number NM_001191), mouse (Mus musculus; NM_001289716), chimpanzee (Pan troglodytes; XM_003316898), macaque (Macaca mulatta; NM_001260717), horse (Equus caballus; XM_005604554), cow (Bos taurus; XM_005214498), dog (Canis lupus familiaris; XM_005634675), mesocricetus (Mesocricetus auratus; XM_005086059), rat (Rattus; XM_006235265); Bcl2A1 3′UTR: human (Homo sapiens; NM_004049), mouse (Mus musculus; NM_009742), chimpanzee (Pan troglodytes; XM_003314817), horse (Equus caballus; XM_001487956), rat (Rattus; NM_133416); RELB 3′UTR: human (Homo sapiens; NM_006509), mouse (Mus musculus; NM_009046), chimpanzee (Pan troglodytes; XM_512742), dog (Canis lupus familiaris; XM_005616459), cow (Bos taurus; XM_002695167.2), rat (Rattus; XM_006223312); BIRC3 3′UTR: human (Homo sapiens; NM_182962.1), mouse (Mus musculus; XM_006509829.1), chimpanzee (Pan troglodytes; XM_001151965), macaque (Macaca fascicularis; XM_005579456.1), gorilla (Gorilla gorilla; XM_004052026), rat (Rattus; NM_023987); Bcl3 3′UTR: human (Homo sapiens; NM_005178), mouse (Mus musculus; NM_003601), chimpanzee (Pan troglodytes; XM_003953521.1), macaque (Macaca mulatta; XM_001109319.2), dog (Canis lupus familiaris; XM_005616874.1), swine (Sus scrofa; XM_003481883.2), rat (Rattus; NM_001109422). Stem-loop sequence conservation analysis was performed by using DNAMAN software. The stem-loop structure was predicted through RNAfold web server (//mrna.tbi.univie.ac.at/).

FIGS. 13A-D. MCPIP1 inhibits MDA-MB-231 tumor progression. (FIG. 13A) The flowchart of in vivo tumor study. 5×10⁶ cells were injected s.c. into mammary glands of NSG mice (day 0). Dox water was provided from day 38 to day 54. (FIG. 13B) Tumors isolated from NSG mice received MDA-MB-231/Tet-On cells at 16 days after drinking dox water (n=8/group). (FIG. 13C) Representative images of excised lungs from NSG mice received dox water or control group (n=8). The metastatic foci were indicated with arrow. (FIG. 13D) After drinking dox water for 4 days (day 42), several tumor-bearing NSG mice were sacrificed and tumors collected for H&E staining. Scale bar: 100 μm.

FIGS. 14A-D. MCPIP1 suppresses 4T1 tumor growth and metastasis in NSG mice. (FIG. 14A) Flowchart for 4T1 tumor study. 0.05×10⁶ 4T1/Tet-On cells were inoculated s.c. into mammary glands of NSG mice (day 0). Dox water was given 12 days later (day 12) and continued until the end of study (day 20). (FIG. 14B) 4T1 tumor growth in NSG mice (n=5/group). The x-axis represents days after tumor injection. The y-axis represents tumor volume (mm₃n=L×W×H; V, volume; W, width; H, high). (FIG. 14C) Survival of 4T1 tumor-bearing mice. On the 20th day, all mice drinking normal water were dead or defined as dead based on syndrome, such as breathe weak, unable to drink or eat. All mice fed with dox water at either day 0 or day 12 were survived. (FIG. 14D) H&E staining of tumor sections harvested at day 20 from mice drinking dox water (lower, Dox+) and normal water group (upper, Dox−). Scale bar, 100 im.

FIGS. 15A-G. MCPIP1 suppresses 4T1 breast tumor growth and metastasis in syngeneic WT Balb/c mice. (FIG. 15A) 0.1×10⁶ 4T1/Tet-On tumor cells were inoculated s.c. into mammary glands of wild-type female Balb/c (n=5/group). The day fed Dox water (1 mg/ml) is indicated with an arrow. (FIG. 15B) Representative images of excised lungs from Balb/c mice received dox water or normal water. The metastatic foci were indicated with arrow. (FIG. 15C) Quantification of the number of metastatic cells in the lungs at 20 days after tumor cell inoculation. The lungs were cut to small pieces and digested with collagenase IV and selected with 6-thioguanine for the metastatic 4T1 tumor cell clones for 2 weeks. (FIG. 15D) Survived 6-thioguanine-resistant 4T1 clones from lungs of the mice fed with Dox water were treated with Dox (500 ng/ml) for 24 hours, followed by taking GFP imaging. (FIG. 15E) H&E staining of tumor sections of mice at day 4 and 10 after providing with Dox water. Scale bar, 100 im. TUNEL staining of tumor tissues of the Balb/c mice fed with or without dox water (FIG. 15F). Quantification of the percentage of TUNEL-positive cells per slides (FIG. 15G).

FIGS. 16A-D. Serum from Dox-drinking mice can induce GFP/MCPIP1 expression in vitro. (FIG. 16A) Body weight of mice that were given dox water and normal water (n=5/group). Serum of mice fed with dox water can induce GFP/MCPIP1 expression in vitro. After fed with dox water for 2-3 days, blood were harvested from caudal vein and serum obtained. The serum was then added into MDA-MB-231/Tet-On cells for GFP expression (FIG. 16B). The percentage of GFP-expressing MDA-MB-231/Tet-On cells after adding serum from mice fed with dox water (FIG. 16C). (FIG. 16D) Confocal fluorescence microscope observation of frozen tissue sections of tumors isolated from NSG mice received MDA-MB-231/Tet-On cells 4 days after drinking dox water.

FIG. 17. Model of MCPIP1-induced apoptosis. Anti-apoptotic genes, including bcl2l1, birc3, bcl2a1, bcl3 and relb, are transcripted in tumor cells and make tumor cells resistant to apoptosis. MCPIP1 preferentially binds to the stem-loop structure located in the 3′UTR of the anti-apoptotic gene mRNAs via the PIN-like domain. This causes mRNA degradation of the anti-apoptotic genes and tips the balance towards apoptosis, resulting in cell death and tumor suppression.

FIG. 18. Protein sequence of MCPIP1. (SEQ ID NO: 103)

FIG. 19A-F. Expressing MCPIP1 by adenovirus inhibits tumor cell growth. (FIG. 19A) MCPIP1 expression was measured by qRT-PCR in various types of human tumor cells (DU145, prostate cancer cells; Huh7, liver cancer cells; SW620, colon cancer cells; NCI-H23 lung cancer cells; and T47D, breast cancer cells), and compared to their respect normal cells (PZ-HPV-7, normal prosatate cells; IHH, normal liver cells; CCD-18Co, normal colon cells; MRC5, normal lung cells, MCF10A, normal breast cells). (FIG. 19B) MCPIP1/Adenovirus (MCPIP1/Ad) and its control empty adenovirus (Ctrl/Ad) were used to infect various types of tumor cells with the same amount of virus, respectively. The numbers of live cells were counted at different times as indicated. The levels of MCPIP1 expressed by the adenovirus were inserted in each figure. (FIG. 19C) MDA-MB-231 tumor cells were inolculated into mammary glands of the NSG mice. When tumor sizes reached ˜5 mm in diameter, 3×10⁸ pfu of MCPIP1/Ad or Control/Ad were injected into tumors for contineously four days, with 1.2×10⁹ pfu adenovirus in total. The sizes of tumors were measured. Each group has five mice. (FIG. 19D) human T lymphoma cells CEM were used to test the effects of MCPIP1 on cell survival. CEM cells were infected with MCPIP1-shRNA/lentivirus and Sramble-shRNA/lentivirus, followed by measuring live cells at different times as indicated. (FIG. 19E) CEM tumor cells were generated to express MCPIP1 protein with a Tet-on inducible system. The numbers of live CEM cells were counted after adding 500 ng/ml of Dox. (FIG. 19F) CEM/Tet-On cells were injected s.c. into the NSG mice. Dox water was provided 20 days after tumor cell injection. Tumor volume (mm₃=L×W×H; V, volume; W, width; H, high) was recorded. Each group has five mice.

### DETAILED DESCRIPTION OF THE EMBODIMENTS

The ability to evade apoptosis is a hallmark of cancer. Here, the inventors identify monocyte chemotactic protein induced protein 1 (MCPIP1) as a potent tumor suppressor that induces apoptosis of breast tumor cells by selectively enhancing mRNA decay of anti-apoptotic gene transcripts, including Bcl2L1, Bcl2A1, RelB, Birc3, and Bcl3. MCPIP1 functions through physical interaction between the PIN domain of MCPIP1 and a stem-loop structure in the 3′UTRs of these anti-apoptotic transcripts. By surveying a gene array data set derived from the excised breast tumors of 251 patients (Miller et al., 2005), the inventors found that low MCPIP1 levels correlated strongly with poor survival of breast cancer patients over 13 years of follow up. These results suggest that MCPIP1 is a biomarker for prognosis and a potent tumor suppressor involved in regulating apoptotic pathway through suppression of anti-apoptotic gene expression. Moreover, ectopic expression of MCPIP1 causes tumor cell apoptosis while knocking-down MCPIP1 expression enhances tumor cell proliferation. Furthermore, MCPIP1 induction in vivo completely abrogates established tumors and significantly reduced metastasis. Importantly, low MCPIP1 levels in tumors are strongly associated with poor survival of breast cancer patients over 13 years of follow up. Moreover, expressing MCPIP1 by adenovirus inhibits growth of various types of tumor cells. Collectively, these results indicate that MCPIP1 is a new tumor suppressor through inducing tumor apoptosis by tipping the balance of pro-apoptotic and anti-apoptotic genes. These and other aspects of the disclosure are discussed below.

### I. MCPIP1

A. MCPIP1 Function

According to the present disclosure, MCPIP1 will be utilized as a tumor suppressor. This molecule is capable of suppressing tumor phenotypes and oncogenic functions in various cancers. In addition to the entire MCPIP1 molecule, the present disclosure also relates to fragments of the polypeptide that may retain the tumor suppressing activity, including synthetic peptides. MCPIP1 and fragments thereof may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

MCPIP1, a Cys-Cys-Cys-His-type zinc finger protein encoded by the zc3h12a gene, was initially discovered as the most highly induced mRNA by monocyte chemotactic protein-1 (MCP-1) in human peripheral blood monocytes (Zhou et al., 2006). MCPIP1 is rapidly induced in macrophages upon stimulation with proinflammatory molecules, such as TNF, IL-1β, and LPS (Liang et al., 2008, Huang et al., 2013 and Matsushita et al., 2009) MCPIP1 has RNase activity and inhibits the expression of proinflammatory cytokines (IL-1, IL-6, and IL-12) by binding to their 3′UTRs for mRNA degradation.

MCPIP1 is also named as Regnase-1 based on the RNase activity (Matsushita et al., 2009). In addition, MCPIP1 can act like a brake for T cell activation (Uehata et al., 2013). Therefore, MCPIP1 is believed to be a key negative regulator involved in the control of inflammation and maintenance of homeostasis. Mice deficient of MCPIP1 develop a complex phenotype, including autoimmune disorders, anemia, and a severe inflammatory response (Matsushita et al., 2009 and Liang et al., 2010). It has been reported recently that MCPIP1 degrades viral RNA and thus acts as a host defense against virus infection (Lin et al., 2013, Liu et al., 2013 and Lin et al., 2014). MCPIP1 also involves in controlling cytokines-induced endothelial inflammation (Qi et al., 2010) and inducing endothelial dysfunction (He et al., 2013). However, prior to this report, it was unknown whether MCPIP1 plays a role in cancer progression and apoptosis evasion.

B. Purification of Proteins

It will be desirable to purify MCPIP1 or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present disclosure concern the purification, and in particular embodiments, the substantial purification, of protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A particular method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present disclosure is discussed below.

### II. NUCLEIC ACIDS

The present disclosure also provides, in another embodiment, genes encoding MCPIP1. A gene for the human MCPIP1 molecule has been identified. The present disclosure is not limited in scope to this gene, however, as one of ordinary skill in the art could readily identify related homologs in various other species (e.g., mouse, rat, rabbit, dog. monkey, gibbon, chimp, ape, baboon, cow, pig, horse, sheep, cat and other species).

In addition, it should be clear that the present disclosure is not limited to the specific nucleic acids disclosed herein. As discussed below, a “MCPIP1 gene” may contain a variety of different bases and yet still produce a corresponding polypeptide that is functionally indistinguishable from, and in some cases structurally identical to, the human gene disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present disclosure may prove useful in the context of screening for agents that induce, repress, inhibit, augment, interfere with, block, abrogate, stimulate or enhance the function of MCPIP1.

A. Nucleic Acids Encoding MCPIP1

Nucleic acids according to the present disclosure may encode an entire MCPIP1 protein, a domain of MCPIP1 that expresses a tumor suppressing function, or any other fragment of the MCPIP1 sequences set forth herein. The nucleic acid may be derived from genomic DNA, i.e., cloned directly from the genome of a particular organism. In particular embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present disclosure may be used as molecular weight standards in, for example, gel electrophoresis. The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. It also is contemplated that a given MCPIP1 from a given species may be represented by natural variants that have slightly different nucleic acid sequences but, nonetheless, encode the same protein.

As used in this application, the term “a nucleic acid encoding a MCPIP1” refers to a nucleic acid molecule that has been isolated free of total cellular nucleic acid. The term “functionally equivalent codon” is used herein to refer to codons that encode the same amino acid, such as the six codons for arginine or serine, and also refers to codons that encode biologically equivalent amino acids, as discussed in the following pages.

The DNA segments of the present disclosure include those encoding biologically functional equivalent MCPIP1 proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present disclosure also encompasses DNA segments that are complementary, or essentially complementary, to an MCPIP1 encoding sequence. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the coding region of MCPIP1 under relatively stringent conditions such as those described herein. Such sequences may encode the entire MCPIP1 protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present disclosure is in the search for genes related to MCPIP1 or, more particularly, homologs of MCPIP1 from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present disclosure is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is used, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double-stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Vectors for Cloning, Gene Transfer and Expression

Within certain embodiments, expression vectors are employed to express the MCPIP1 polypeptide product, which can then be purified for various uses. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. One example is the native MCPIP1 promoter. Those of skill in the art of molecular biology generally know the use of promoters, enhancers, and cell type combinations for protein expression, for example, see Sambrook et al. (1989), incorporated herein by reference. The promoters employed may be constitutive, tissue-specific (including for particular type sof cancer cells), inducible, and/or useful under the appropriate conditions to direct high level expression of the introduced DNA segment. The promoter may be heterologous or endogenous.

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present disclosure. Some such promoters are set forth in Tables 1 and 2.

TABLE 1
Candidate Tissue-Specific Promoters for Cancer Gene Therapy

| | | Normal cells in |
|---|---|---|
| Tissue-specific | Cancers in which | which promoter is |
| promoter | promoter is active | active |
| Carcinoembryonic | Most colorectal | Colonic mucosa; gastric |
| antigen (CEA)* | carcinomas; 50% of | mucosa; lung epithelia; |
| | lung carcinomas; | eccrine sweat glands; |
| | 40-50% of gastric | cells in testes |
| | carcinomas; most | |
| | pancreatic | |
| | carcinomas; many | |
| | breast carcinomas | |
| Prostate-specific | Most prostate | Prostate epithelium |
| antigen (PSA) | carcinomas | |
| Vasoactive | Majority of | Neurons; lymphocytes; |
| intestinal | non-small cell | mast cells; eosinophils |
| peptide (VIP) | lung cancers | |
| Surfactant protein | Many lung | Type II pneumocytes; |
| A (SP-A) | adenocarcinomas | Clara |
| | cells | |
| Human achaete-scute | Most small cell | Neuroendocrine cells |
| homolog (hASH) | lung cancers | in lung |
| Mucin-1 (MUC1)** | Most | Glandular epithelial |
| | adenocarcinomas | cells in breast and in |
| | (originating from | respiratory, |
| | any tissue) | gastrointestinal, and |
| | | genitourinary tracts |
| Alpha-fetoprotein | Most hepatocellular | Hepatocytes (under |
| | carcinomas; | certain conditions); |
| | possibly many | testis |
| | testicular | |
| | cancers | |
| Albumin | Most hepatocellular | Hepatocytes |
| | carcinomas | |
| Tyrosinase | Most melanomas | Melanocytes; astrocytes; |
| | | Schwann cells; some |
| | | neurons |
| Tyrosine-binding | Most melanomas | Melanocytes; astrocytes, |
| protein (TRP) | | Schwann cells; some |
| | | neurons |
| Keratin 14 | Presumably many | Keratinocytes |
| | squamous cell | |
| | carcinomas | |
| | (e.g . . . , | |
| | Head and neck | |
| | cancers) | |
| EBV LD-2 | Many squamous cell | Keratinocytes of upper |
| | carcinomas of head | digestive Keratinocytes |
| | and neck | of upper digestive |
| | | tract |
| Glial fibrillary | Many astrocytomas | Astrocytes |
| acidic protein | | |
| (GFAP) | | |
| Myelin basic | Many gliomas | Oligodendrocytes |
| protein (MBP) | | |
| Testis-specific | Possibly many | Spermatazoa |
| angiotensin- | testicular cancers | |
| converting enzyme | | |
| (Testis-specific | | |
| ACE) | | |
| Osteocalcin | Possibly many | Osteoblasts |
| | osteosarcomas | |

TABLE 2
Candidate Promoters for Tissue-Specific Targeting of Tumors

| | Cancers in which | Normal cells in which |
|---|---|---|
| Promoter | Promoter is active | Promoter is active |
| E2F-regulated | Almost all cancers | Proliferating cells |
| promoter | | |
| HLA-G | Many colorectal | Lymphocytes; monocytes; |
| | carcinomas; many | spermatocytes; trophoblast |
| | melanomas; possibly | |
| | many other cancers | |
| FasL | Most melanomas; many | Activated leukocytes: |
| | pancreatic carcinomas; | neurons; endothelial cells; |
| | most astrocytomas | keratinocytes; cells in |
| | possibly many | immunoprivileged tissues; |
| | other cancers | some cells in lungs, |
| | | ovaries, |
| | | liver, and prostate |
| Myc-regulated | Most lung carcinomas | Proliferating cells (only |
| promoter | (both small cell and | some cell-types): mammary |
| | non-small cell); | epithelial cells (including |
| | most colorectal | non-proliferating) |
| | carcinomas | |
| MAGE-1 | Many melanomas; some | Testis |
| | non-small cell lung | |
| | carcinomas; some | |
| | breast carcinomas | |
| VEGF | 70% of all cancers | Cells at sites of |
| | (constitutive | neovascularization (but |
| | overexpression in | unlike in tumors, |
| | many cancers) | expression is transient, |
| | | less strong, and never |
| | | constitutive) |
| bFGF | Presumably many | Cells at sites of ischemia |
| | different cancers, | (but unlike tumors, |
| | since bFGF | expression is transient, |
| | expression is | less strong, and never |
| | induced by ischemic | constitutive) |
| | conditions | |
| COX-2 | Most colorectal | Cells at sites of |
| | carcinomas; many | inflammation |
| | lung carcinomas; | |
| | possibly many other | |
| | cancers | |
| IL-10 | Most colorectal | Leukocytes |
| | carcinomas; many | |
| | lung carcinomas; | |
| | many squamous cell | |
| | carcinomas of head | |
| | and neck; possibly | |
| | many other cancers | |
| GRP78/BiP | Presumably many | Cells at sites of ishemia |
| | different cancers, | |
| | since GRP7S | |
| | expression is | |
| | induced by | |
| | tumor-specific | |
| | conditions | |
| CarG elements | Induced by | Cells exposed to ionizing |
| from Egr-1 | ionization radiation, | radiation; leukocytes |
| | so conceivably most | |
| | tumors upon | |
| | irradiation | |

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

2. Splice Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference.)

3. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, the terminator may comprise a signal for the cleavage of the RNA, and the terminator signal may promote polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

4. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Particular embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

5. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

6. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

7. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors.

In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present disclosure comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a qp sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The ψ sequence is required for the packaging of the adenoviral genome.

A common approach for generating adenoviruses for use as a gene transfer vector is the deletion of the E1 gene (E1-), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1-, replication-deficient virus is then proliferated in a “helper” cell line that provides the E1 polypeptides in trans (e.g., the human embryonic kidney cell line 293). Thus, in the present disclosure it may be convenient to introduce the transforming construct at the position from which the E1-coding sequences have been removed. However, the position of insertion of the construct within the adenovirus sequences is not critical to the disclosure. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. No. 5,670,488; U.S. Pat. No. 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1997) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al. (1990), describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the disclosure. The adenovirus may be of any of the 42 different known serotypes and/or subgroups A-F. Adenovirus type 5 of subgroup C is a particular starting material in order to obtain the conditional replication-defective adenovirus vector for use in the present disclosure. This is because adenovirus type 5 is a human adenovirus about which a great deal of biochemical and genetic information is known, and it has historically been used for most constructions employing adenovirus as a vector.

As stated above, the typical vector according to the present disclosure is replication defective and will not have an adenovirus E1 region. Adenovirus growth and manipulation is known to those of skill in the art, and exhibits broad host range in vitro and in vivo (U.S. Pat. No. 5,670,488; U.S. Pat. No. 5,932,210; U.S. Pat. No. 5,824,544). This group of viruses can be obtained in high titers, e.g., 10⁹ to 10¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic, bladder (Irie et al., 1999), head and neck (Blackwell et al., 1999), breast (Stewart et al., 1999), lung (Batra et al., 1999) and ovarian (Vanderkwaak et al., 1999).

Retroviral Vectors.

In certain embodiments of the disclosure, the use of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present disclosure may be genetically modified in such a way that some of the structural, infectious genes of the native virus have been removed and replaced instead with a nucleic acid sequence to be delivered to a target cell (U.S. Pat. No. 5,858,744; U.S. Pat. No. 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present disclosure are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. No. 5,955,331; U.S. Pat. No. 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present disclosure (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery include a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; White et al., 1999; Case et al., 1999). For example, HIV-based vectors have been used to infect non-dividing cells such as neurons (Miyatake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado et al., 1999; Engel and Kohn, 1999), cancer (Clay et al., 1999), cerebrovascular disease (Weihl et al., 1999) and hemophilia (Kay, 1998).

Herpesviral Vectors.

Herpes simplex virus (HSV) type I and type II contain a double-stranded, linear DNA genome of approximately 150 kb, encoding 70-80 genes. Wild-type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Garrido et al., 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or alpha genes, Early (E) or beta genes and Late (L) or gamma genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. No. 5,879,934; U.S. Pat. No. 5,851,826, each specifically incorporated herein by reference in its entirety). One IE protein, Infected Cell Polypeptide 4 (ICP4), also known as alpha 4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B-cell malignancies (Suzuki et al., 1998), ovarian cancer (Wang et al., 1998) and Duchenne muscular dystrophy (Huard et al., 1997).

Adeno-Associated Viral Vectors.

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

Lentiviral Vectors.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR contains all other cis-acting sequences necessary for viral replication. Lentiviruses have additional genes including vif vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as the STAT-1α gene in this disclosure, into that packaging cell yields a producer cell which releases infectious viral particles carrying the foreign gene of interest. The env is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1α encoding polynucleotide sequence herein, is linked operably to a regulatory nucleic acid sequence. The heterologous sequence may be linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc., and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gin synthetase or ADA, followed by selection in the presence of the appropriate drug and isolation of clones. The selectable marker gene can be linked physically to the packaging genes in the construct.

Lentiviral transfer vectors Naldini et al. (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., 1997). Thus, in the present disclosure, one may graft or transplant cells infected with the recombinant lentivirus ex vivo, or infect cells in vivo.

Other Viral Vectors.

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

In certain embodiments, vaccinia viral vectors are contemplated for use in the present disclosure. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g., P-450 (U.S. Pat. No. 5,506,138). The preparation, growth and manipulation of vaccinia viruses are described in U.S. Pat. No. 5,849,304 and U.S. Pat. No. 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al., 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, it has a wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

Chimeric Viral Vectors.

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1999; Caplen et al., 1999) and adenoviralladeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

8. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection:

In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation.

In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

Calcium Phosphate.

In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading.

Additional embodiments of the present disclosure include the introduction of a nucleic acid by direct sonic loading. LTK-fibroblasts have been transfected with the thymidine kinase gene by sonication loading (Fechheimer et al., 1987).

Liposome-Mediated Transfection.

In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor Mediated Transfection:

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

D. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr that confers resistance to; gpt, that confers resistance to mycophenolic acid; neo, that confers resistance to the aminoglycoside G418; and hygro, that confers resistance to hygromycin.

### III. DIAGNOSING CANCERS WITH DEFICIENCIES IN MCPIP1

MCPIP1 antibodies or MCPIP1 nucleic acids may be employed as a diagnostic or prognostic indicator of cancer. More specifically, point mutations, deletions, insertions or regulatory pertubations relating to the reduction in expression or activity of MCPIP1 may cause or promote cancer development, cause or promote tumor progression at a primary site, and/or cause or promote tumore metastasis.

A. Genetic Diagnosis

One embodiment of the instant disclosure comprises a method for detecting variation in the expression of MCPIP1, or in the structure of the MCPIP1 gene or gene product. Also contemplated are epigenetic modifications, such as methylation of promoter regions. This may comprises determining that level of MCPIP1 or determining specific alterations in the expressed product. Such cancers with MCPIP1 defects may include brain (glioblastomas, medulloblastoma, astrocytoma, oligodendroglioma, ependymomas), lung, liver, spleen, kidney, pancreas, small intestine, blood cells, lymph node, colon, breast, endometrium, stomach, prostate, testicle, ovary, skin, head and neck, esophagus, bone marrow, blood or other tissue.

The biological sample can be any tissue or fluid that can contain cells. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, nipple aspirates, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have MCPIP1-related pathologies. In this way, it is possible to correlate the amount or structure of MCPIP1 detected with various clinical states.

“Alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations or epigenetic modifications in and outside the coding region also may affect the amount of MCPIP1 produced, both by altering the transcription of the gene or in destabilizing or otherwise altering the processing of either the transcript (mRNA) or protein.

A cell takes a genetic step toward oncogenic transformation when one allele of a tumor suppressor gene is inactivated due to inheritance of a germline lesion or acquisition of a somatic mutation. The inactivation of the other allele of the gene usually involves a somatic micromutation or chromosomal allelic deletion that results in loss of heterozygosity (LOH). Alternatively, both copies of a tumor suppressor gene may be lost by homozygous deletion.

It is contemplated that other mutations in the MCPIP1 gene may be identified in accordance with the present disclosure. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

B. Immunodiagnosis

Antibodies of the present disclosure can be used in characterizing the MCPIP1 content of healthy and diseased tissues, through techniques such as immunohistochemistry, ELISAs and Western blotting. This may provide a screen for the presence or absence of malignancy or as a predictor of future cancer.

The use of antibodies of the present disclosure, in an ELISA assay is contemplated. For example, anti-MCPIP1 antibodies are immobilized onto a selected surface, in particular a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface. After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for MCPIP1 that differs the first antibody. Appropriate conditions include diluting the sample with diluents such as BSA, bovine gamma globulin (BGG) and phosphate buffered saline (PBS)/Tween®. These added agents also tend to assist in the reduction of nonspecific background. The layered antiserum is then allowed to incubate for from about 2 to about 4 hr, at temperatures on the order of about 25° to about 27° C. Following incubation, the antisera-contacted surface is washed so as to remove non-immunocomplexed material. A particular washing procedure includes washing with a solution such as PBS/Tween®, or borate buffer.

To provide a detecting means, the second antibody may have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present disclosure will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

Immunohistochemistry may be useful according to the present disclosure as well. Such a detection method would require a biopsy of a sample or mastectomy from an individual suffering or suspectd of suffering from cancer. This technique involves testing of both fresh-frozen and formalin-fixed, paraffin-embedded tissue blocks prepared from study by immunohistochemistry (IHC). For example, each tissue block consists of 50 mg of residual “pulverized” tissue. The method of preparing tissue blocks from these particulate specimens has been successfully used in previous IHC studies of various diagnostic and prognostic factors is well known to those of skill in the art.

Briefly, frozen-sections may be prepared by rehydrating 50 ng of frozen “pulverized” sample at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections containing an average of about 500 intact cells.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

### IV. METHODS OF THERAPY

The present disclosure also involves, in another embodiment, the treatment of cancer. Any type of cancer may be treated, but in particular those where defects in MCPIP1 expression or activity exist. Thus, it is contemplated that a wide variety of tumors may be treated using MCPIP1 therapy, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

It is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Genetic Based Therapies

One of the therapeutic embodiments contemplated by the present inventors is the intervention, at the molecular level, in the events involved in tumorigenesis. Specifically, the present inventors intend to provide, to a cancer cell, an expression construct capable of providing MCPIP1 to that cell. Because the sequence homology between the human, mouse and dog genes, any of these nucleic acids could be used in human therapy, as could any of the gene sequence variants discussed above which would encode the same, or a biologically equivalent polypeptide. The lengthy discussion of expression vectors and the genetic elements employed therein is incorporated into this section by reference. Particular expression vectors are viral vectors such as adenovirus, adeno-associated virus, herpesvirus, vaccinia virus and retrovirus. Also contemplated are liposomally-encapsulated expression vectors.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized. For blood borne cancers, such as leukemias, a systemic route will be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

Autologous bone marrow transplant (ABMT) is an example of ex vivo gene therapy. Basically, the notion behind ABMT is that the patient will serve as his or her own bone marrow donor. Thus, a normally lethal dose of irradiation or chemotherapeutic may be delivered to the patient to kill tumor cells, and the bone marrow repopulated with the patients own cells that have been maintained (and perhaps expanded) ex vivo. Because, bone marrow often is contaminated with tumor cells, it is desirable to purge the bone marrow of these cells. Use of gene therapy to accomplish this goal is yet another way MCPIP1 may be utilized according to the present disclosure.

B. Protein Therapy

Another therapy approach is the provision, to a subject, of MCPIP1 polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

C. Combined Therapy with Immunotherapy, Traditional Chemo- or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present disclosure, it is contemplated that MCPIP1 replacement therapy could be used similarly in conjunction with chemo- or radiotherapeutic intervention. It also may prove effective to combine MCPIP1 gene therapy with immunotherapy, such as with an adjuvant therapy, anti-CTLA4 therapy, or PD1/PDL1 checkpoint blockage, IL-2, Trastuzumab, IDO inhibitors or PLX3397.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with a MCPIP1 expression construct and at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other, within about 6-12 hours of each other, or about 12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either MCPIP1 or the other agent will be desired. Various combinations may be employed, where MCPIP1 is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A
B/A/B/A B/A/A/B B/B/B/A A/A/A/B B/A/A/A
A/B/A/A A/A/B/A A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The disclosure also encompasses the use of a combination of one or more DNA damaging agents, whether radiation-based or actual compounds, such as the use of X-rays with cisplatin or the use of cisplatin with etoposide. In certain embodiments, the use of cisplatin in combination with a MCPIP1 expression construct is particularly contemplated.

In treating cancer according to the disclosure, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or, cisplatin. The agent may be prepared and used as a combined therapeutic composition, or kit, by combining it with a MCPIP1 expression construct, as described above.

Agents that directly cross-link nucleic acids, specifically DNA, are envisaged to facilitate DNA damage leading to a synergistic, antineoplastic combination with MCPIP1. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the local or regional delivery of MCPIP1 expression constructs to patients with cancer will be a very efficient method for treating the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining MCPIP1 therapies with chemo- and radiotherapies, it also is contemplated that combination with other gene therapies will be advantageous. For example, targeting of MCPIP1 and p53 mutations at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf erb, src, fms, jun, trk, ret, gsp, hst, bcl and abl.

It also should be pointed out that any of the foregoing therapies may prove useful by themselves in treating a MCPIP1-related cancer. In this regard, reference to chemotherapeutics and non-MCPIP1 gene therapy in combination should also be read as a contemplation that these approaches may be employed separately.

E. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the specific methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. KITS

According to the present disclosure, there are provided kits for detecting MCPIP1 mutations and MCPIP1 expression. The kit of the present disclosure can be prepared by known materials and techniques which are conventionally used in the art. Generally, kits comprises separate vials or containers for the various reagents, such as probes, primers, enzymes, antibodies, etc. The reagents are also generally prepared in a form suitable for preservation by dissolving it in a suitable solvent. Examples of a suitable solvent include water, ethanol, various buffer solutions, and the like. The various vials or containers are often held in blow-molded or injection-molded plastics.

### VI. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Materials & Methods

Mice.

6˜8 week old female Balb/c mice and NSG mice were obtained from The Jackson Laboratories and respectively housed in cages with filter tops in a laminar flow hood, fed food and acid water ad libitum and in pathogen-free condition. All experimental procedures were performed with the approval of the IACUC at Saint Louis University.

Cells and Plasmids.

MDA-MB-231, MDA-MB-453, MCF-10A, MCF-12A, 4T1, Ts/A, and HEK293 cells, as well as DU145, Huh7, SW620, NCI-H23, T47D, PZ-HPV-7, IHH, CCD-18Co, and CEM-ss cells were obtained from ATCC and maintained in DMEM with 10% FBS. Mouse mammary gland epithelial cells FSK4 and CommD were kindly provided by Janet S Butel and cultured as described (Hollmann et al., 2001). Isogenic tumorigenic line 67NR, 168FARN, 4TO7, 66cl4 and 4T1 were kindly provided by Yibin Kang and cultured as described (Lu et al., 2010). Retro-X Tet-On 3G inducible MCPIP1 cell lines were established in 4T1 and MDA-MB-231 cells according to the manufacturer's instruction (Clontech). A set of luciferase-expressing 3′UTR reporter plasmids (BCL2L1_{3′UTR}, BCL2A1_{3′UTR}, BIRC3_{3′UTR}, RELB3_{3′UTR}, BCL3_{3′UTR}, and β-ACTIN_{3′UTR}) were cloned by inserting their 3′UTRs into the pGL3 control vector (Promega) between XbaI and FseI sites. For stem-loop deletion constructs, mutated and truncated BCL2L1 3′UTR (1-1559 bp) and BIRC3 3′UTR (1-544 bp) were amplified, sequenced, and inserted into pGL3 control vector as described above.

qPCR and PCR Array.

The TaqMan® Human Apoptosis PCR Array (Life Technologies) was used to analyze apoptosis-related gene expression. Total RNA was extracted with Trizol from MCPIP1-expressed and -unexpressed MDA-MB-231 cells, purified using the RNA Cleanup Kit (Fisher Scientific), reverse-transcribed to cDNA, and added to each well of the PCR Array plates combined with TaqMan Master Mix according to the manufacturer's instructions. Data analysis was based on the ΔΔCt method, with normalization to three different housekeeping genes. The genes in the Array and primer sequences used are described in Supplemental Tables 1 and 2.

RNA Immunoprecipitation.

GFP/MCPIP1 fusion protein was induced by Dox for 30 h in MDA-MB-231/Tet-On cells. Then cells were lysed and whole cell lysates pre-cleared with IgG, followed by incubation with anti-GFP antibody at 4° C. for 4 h. The RNA-protein complexes were pulled-down by protein A/G agarose beads and RNA extracted with Trizol, followed by detecting apoptosis-related genes with RT-PCR.

mRNA Stability.

MCPIP1 was induced by Dox in MDA-MB-231/Tet-On cells for 36 h. Then 5 μg/ml of ActD and 5 μg/ml of DRB were added to block de novo RNA synthesis. Total RNA was harvested at indicated time points and mRNA expression analyzed by qRT-PCR. The mRNA levels were normalized to GAPDH mRNA and half-life of the mRNA determined by comparing to the levels of mRNA before adding ActD and DRB.

shRNA Lentivirus.

Three lentiviral shRNAs targeting human MCPIP1 mRNA were purchased from Sigma (St. Louis, Mo.), with #1 targeting MCPIP1 CDS (NM_025079.1-1777slcl), #2 targeting 3′UTR (NM_025079.1-2532scl), and #3 targeting CDS (NM_025079.1-260slcl). A scramble non-targeting shRNA was used as control. MCPIP1-shRNA lentiviral particles were packaged in HEK293T cells by co-transfecting shRNA-MCPIP1-pLKO.1, pCMV-dR8.2, and pMD2.G constructs. 48 h later, supernatants were collected and centrifuged to discard cell debris. For infection, virus supernatants with 1 μg/ml Polybrene were added to MDA-MB-231 and MDA-MB-453 cells for overnight. After two rounds infection, the infected cells were selected with puromycin (2.5 μg/ml) for 2 weeks, followed by experimental analysis.

Adenovirus.

HA-tagged human MCPIP1 was cloned into human adenovirus type5 with the E1 and E3 genomic region deleted. Because E1 is essential for the assembly of the virus particles, this adenovirus produces only replication-incompetent adenovirus. The MCPIP1/Ad was amplified in HEK293-TetR cells. The expression of MCPIP1 has been confirmed by Western blot and the titer of virus (pfu) determined.

Immunofluorescence Staining.

Cells in cover slips were fixed with 4% paraformaldehyde and permeabilized with 0.5% Triton X-100 in PBS. After blocking, cells were probed with primary antibody overnight and then incubated with Alexa Fluor 488- or 594-conjugated secondary antibodies. Nuclei were counterstained with DAPI. The images were taken by confocal fluorescence microscopy.

Immunohistochemistry and TUNEL Staining.

Tumor and lung tissues were stained with hematoxylin and eosin (H&E). Images were taken and analysis using Zeiss Imaging System. For frozen section, tumor tissues were embedded with Tissue-Tek OCT compound (VWR) and slowly submerged into liquid nitrogen. The frozen sections were placed on polylysine-treated glass slides and used for GFP, MCPIP1 immunofluorescent staining, and TUNEL staining (Roche).

Luciferase Reporter Assays.

Luciferase assay was performed as described previously (Qian et al., 2011). All transfections were performed in triplicate and repeated at least three times. pGL3 luciferase reporter plasmids containing 3′UTRs of different genes were transfected into HEK293 cells along with Flag-tagged MCPIP1, Flag-ΔZF (zinc finger), Flag-ΔPIN (D141N) and Flag-control plasmids. The luciferase activity was measured 36 h after transfection using a dual-luciferase reporter assay system (Promega).

6-Thioguanine Lung Metastasis Single Clone Selection.

Lungs of tumor-bearing mice were minced and digested with 100 U/ml Collagenase IV (Sigma). Digested lung tissues were passed through a 70-μm strainer. Filtered cell suspension was serially diluted in a 1:10 ratio and plated into 15 cm plates. 60 μM of 6-thioguanine (Sigma) was added for selection of drug-resistant 4T1 tumor cells for 2 weeks. After counted the number of colonies, Dox was added to induce GFP/MCPIP1 expression.

Statistical Analysis.

All data are presented as mean plus SD. Unless indicated otherwise, statistical analysis was performed with the Student's t test. Statistical significance was defined as a p value of <0.05.

Tumor Models.

1×10⁵ 4T1/Tet-On tumor cells in 100 μl phosphate-buffered saline was injected subcutaneously (s.c.) into the abdominal mammary glands of female Balb/c aged 6-8 weeks old. 3×10⁶ MDA-MB-231/Tet-On tumor cells in 100 μl phosphate-buffered saline was injected subcutaneously (s.c.) into the abdominal mammary glands of NSG mice aged 6-8 weeks old. Dox-containing water (1 μg/ml) was fed as indicated. Tumor volume was calculated by the formula length×width×high (mm₃).

Antibodies and Reagents.

Polyclonal rabbit anti-MCPIP1 (sc-136750), anti-GFP (sc-8334), anti-caspase3 (sc-7148), and normal rabbit IgG (sc-2027) were from Santa Cruz (Santa Cruz, Calif.). Polyclonal rabbit anti-PARP1 (9542S) antibody was from Cell Signaling Technology. Monoclonal β-actin (A2066) and anti-Flag (F3165) antibodies were from Sigma. Alexa Fluor® 488 (green), Alexa Fluor® 594 (red) anti-rabbit secondary antibodies, and DAPI (D1306) were from Invitrigen. Doxycycline hydrochloride (D3447) and Thiazolyl Blue Tetrazolium Bromide (MTT) (M2128), Actinomycin D (Act D, A1410), and 5,6-Dichlorobenzimidazole 1-β-D-ribofuranoside (DRB, D1916) were from Sigma-Aldrich (St. Louis, USA). G418 (G8168) and puromycin (P8833) were also from Sigma. Protein G PLUS-Agarose beads were from Santa Cruz Biotechnology. APC Annexin V (640920) was from BioLegend.

RNA ChIP.

RNA-ChIP assays were performed similar to RNA-IP with modifications. In brief, Dox was added to MDA-MB-231/Tet-on cells to induce GFP/MCPIP1 fusion protein expression for 30 h. Then, cells were cross-linked for 10 min by addition of 37% formaldehyde to a final concentration of 1%. Glycine was added to a final concentration of 125 mM to quench crosslinking. The cells were collected at 1500 rpm for 5 min. Cell pellet was washed with cold PBS, re-suspended in 500 μl of polysome lysis buffer, and placed on ice for 5 min. Cell lysates were collected by centrifugation at 10,000 g for 10 min at 4° C., and re-suspended in 500 μl of polysome lysis buffer. Then, the lysates were sonicated and pre-cleared with rabbit IgG-coated beads to remove non-specific binding. Pre-cleared lysates were used for IP with anti-GFP antibody-coated beads or rabbit IgG-coated beads at 4° C. for 4 h. Next, 100 μl supernatants were reserved as Input. Each immune complex was washed five times with ice-cold NT2 buffer. RNA was isolated with Trizol reagent, and resuspended in 50 μl of RNase-free water, followed by DNase I treatment and detection of binding 3′UTRs by RT-PCR.

Western Blotting.

Cells were collected by scraping and lysed with a modified RIPA buffer containing PMSF and a protease inhibitor cocktail (Roche). The concentration of total protein was measured by standard BCA method. Equal amount of cell lysates was subjected to electrophoresis by SDS-PAGE and transferred onto a polyvinylidene membrane. The membrane was then blocked with 5% fat-free milk and incubated with primary antibody overnight. The band was detected with HRP-conjugated secondary antibodies using ECL chemiluminescent detection method. The intensity of the band was measured using ImageJ software.

RNA-EMSA and Supershift.

The single strand RNA oligonucleotides with wild-type sequence and mutant were custom synthesized (IDT) and 3′ end-labeled with biotin using an RNA 3′ End Biotinylation Kit (Pierce) according to the company's instruction. An aliquot of the labeled WT RNA probe (12.5 pmol) was heated at 90-degree for 5 min and then quickly cooled down on ice to disrupt the RNA folding. 20 fmol of the biotin-labeled WT and other probes were incubated with 15 μg of cell lysates extracted from Dox-stimulated MDA-MB-231/Tet-On cells for 30 min at room temperature in the presence of 20 μl of binding buffer containing 10 mM Tris, 50 mM KCl, 1 mM Dithiothreitol (DTT) (pH 7.5), 50 ng/μl Poly (dI.dC). 5 μl of 5× loading buffer was added to the mixtures. RNA complexes were resolved by 6% non-denatured polyacrylamide gel electrophoresis, and the gel transferred onto a nylon membrane (Pierce) and UV-cross linked. The membrane was then subjected to detection by chemiluminescent EMSA kit (Pierce) following the manufacturer's protocol. The supershift assay was performed in the same manner, with 2 μg anti-GFP and anti-MCPIP1 antibodies (Santa Cruz) were added into the reaction.

Sequence Alignments and Stem-Loop Structure Prediction.

For conservation analyses of Bcl2L1, Bcl2A1, RELB, BIRC3 and Bcl3 stem-loop structure, the 3′UTR sequences from different species were extracted from the National Center for Biotechnology Information (NCBI) database: Bcl2L1 3′UTR: human (Homo sapiens; accession number NM_001191), mouse (Mus musculus; NM_001289716), chimpanzee (Pan troglodytes; XM_003316898), macaque (Macaca mulatta; NM_001260717), horse (Equus caballus; XM_005604554), cow (Bos taurus; XM_005214498), dog (Canis lupus familiaris; XM_005634675), mesocricetus (Mesocricetus auratus; XM_005086059), rat (Rattus; XM_006235265); Bcl2A1 3′UTR: human (Homo sapiens; NM_004049), mouse (Mus musculus; NM_009742), chimpanzee (Pan troglodytes; XM_003314817), horse (Equus caballus; XM_001487956), rat (Rattus; NM_133416); RELB 3′UTR: human (Homo sapiens; NM_006509), mouse (Mus musculus; NM_009046), chimpanzee (Pan troglodytes; XM_512742), dog (Canis lupus familiaris; XM_005616459), cow (Bos taurus; XM_002695167.2), rat (Rattus; XM_006223312); BIRC3 3′UTR: human (Homo sapiens; NM_182962.1), mouse (Mus musculus; XM_006509829.1), chimpanzee (Pan troglodytes; XM_001151965), macaque (Macaca fascicularis; XM_005579456.1), gorilla (Gorilla gorilla; XM_004052026), rat (Rattus; NM_023987); Bcl3 3′UTR: human (Homo sapiens; NM_005178), mouse (Mus musculus; NM_003601), chimpanzee (Pan troglodytes; XM_003953521.1), macaque (Macaca mulatta; XM_001109319.2), dog (Canis lupus familiaris; XM_005616874.1), swine (Sus scrofa; XM_003481883.2), rat (Rattus; NM_001109422). Stem-loop sequence conservation analysis was performed by using DNAMAN software. The stem-loop structure was predicted through RNAfold web server (//ma.tbi.univie.ac.at/).

### Example 2—Results

MCPIP1 Expression is Reduced in Breast Tumors and Breast Tumor Cell Lines.

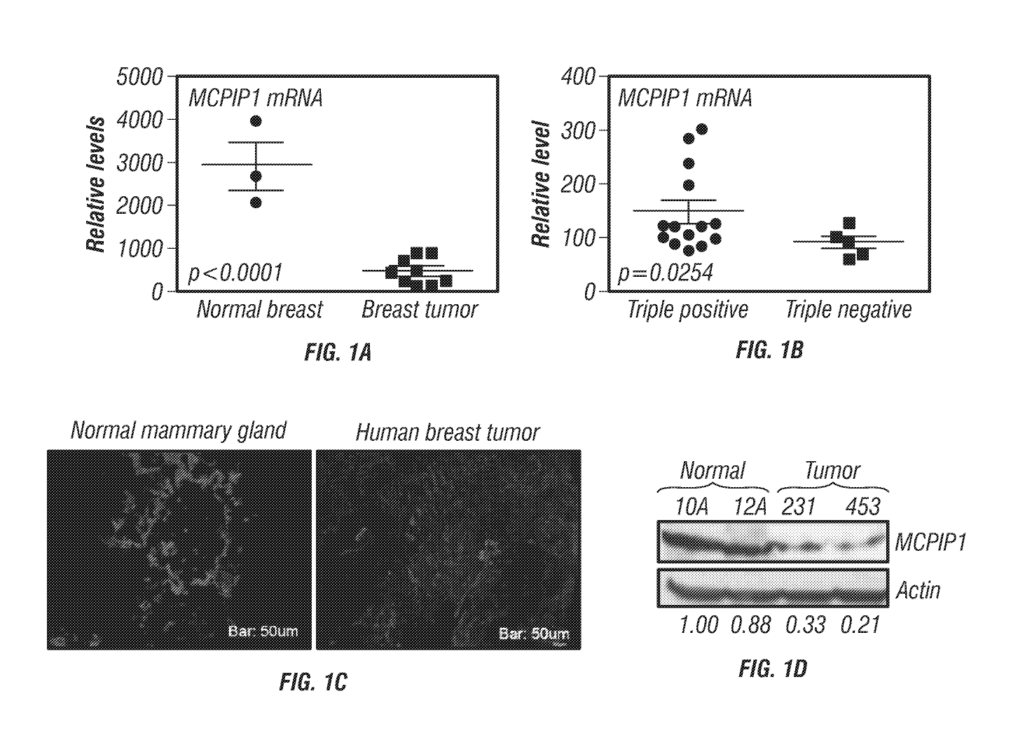

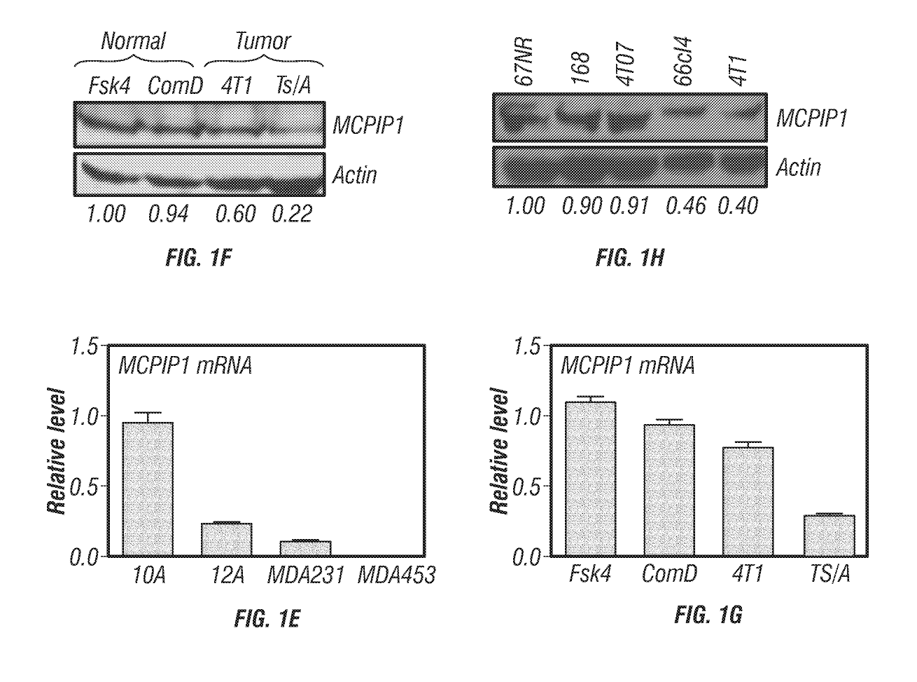

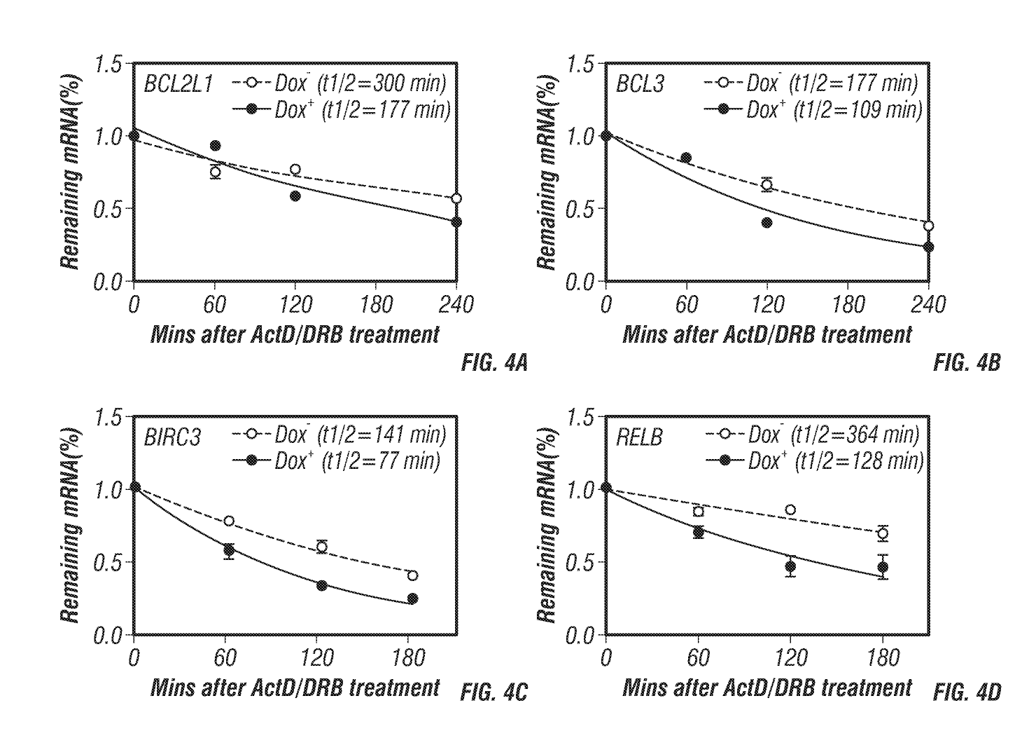

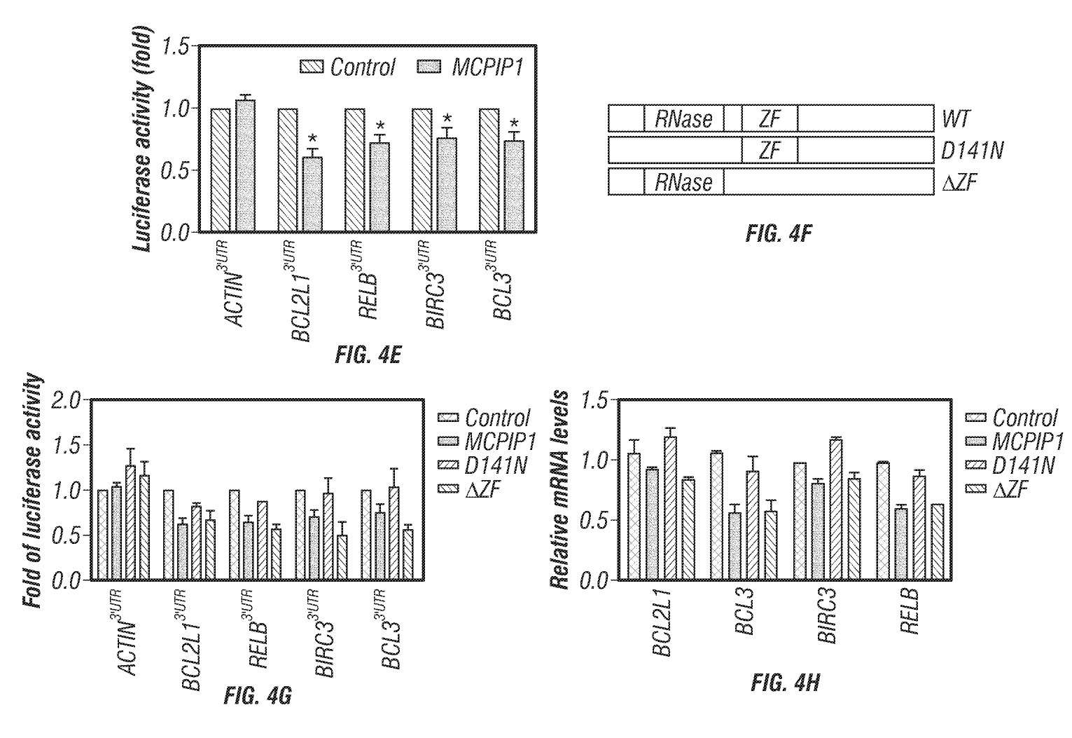

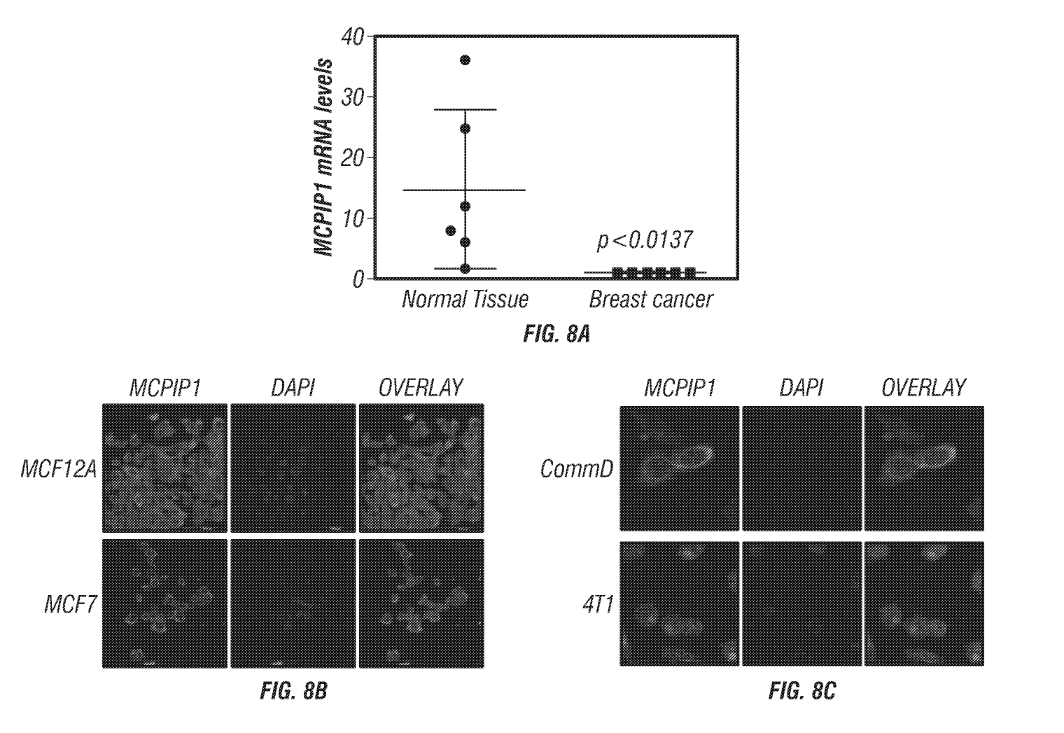

To determine the levels of MCPIP1 in breast tumors, the inventors obtained tumor tissues and the surrounding ‘healthy’ tissues from breast cancer patients undergoing surgery and measured MCPIP1 mRNA expression. MCPIP1 mRNA expression was significantly reduced in tumors compared with ‘healthy’ tissues (FIG. 1A). Since triple negative breast tumors have the worst outcome, the inventors further compared MCPIP1 levels between triple-negative and -positive tumors. MCPIP1 expression was significantly lower in triple negative tumors than it in triple positive tumors (FIG. 1B). Immunofluorescence staining of MCPIP1 protein indicated a lower MCPIP1 levels in breast tumors compared to normal mammary gland tissues (FIG. 1C). MCPIP1 levels were also significantly reduced in breast tumors of nude mice induced with MDA-MB-231 cells (FIG. 8A). To determine if breast tumor cells had MCPIP1 expression repressed, the inventors examined MCPIP1 in human MDA-MB-231 and MDA-MB-453 as well as mouse 4T1 and TS/A breast tumor cell lines and compared them to normal mammary gland epithelial cells. MCPIP1 protein (FIG. 1D) and mRNA (FIG., 1E) were significantly reduced in human breast tumor cells. A similar reduction of MCPIP1 protein (FIG. 1F) and mRNA (FIG. 1G) were also displayed in mouse breast tumor cells. Immunofluorescence staining of MCPIP1 also indicated a lower MCPIP1 levels in MCF7 (FIG. 8B) and 4T1 (FIG. 8C) breast tumor cells, suggesting that the impaired MCPIP1 expression in tumor mass may be due to MCPIP1 reduction in tumor cells. To determine whether MCPIP1 levels were associated with tumor aggressive feature, the inventors measured MCPIP1 in five isogenic tumorigenic lines (67NR, 168FARN, 4T07, 66cl4, and 4T1), with each line having unique tumorigenic feature and increased metastatic capability in order (Lu et al., 2010). The most tumorigenic and metastatic 4T1 cells showed the least levels of MCPIP1 compared to the other metastatic cell lines (FIG. 1H-I), indicating a correlation of MCPIP1 level with tumor progression.

MCPIP1 Induces Apoptosis of Breast Tumor Cells.

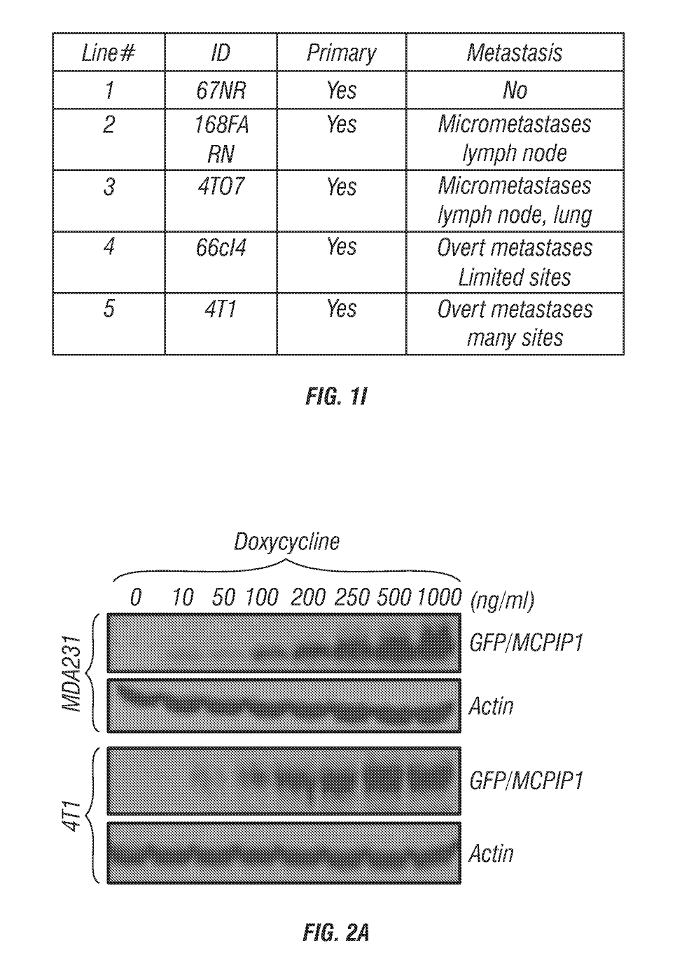

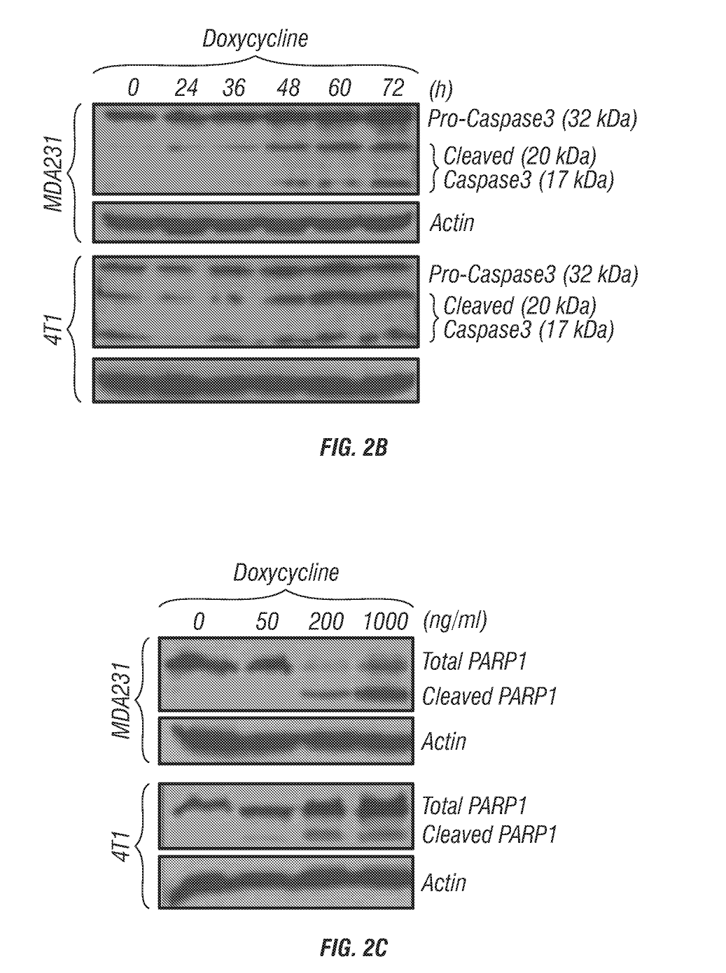

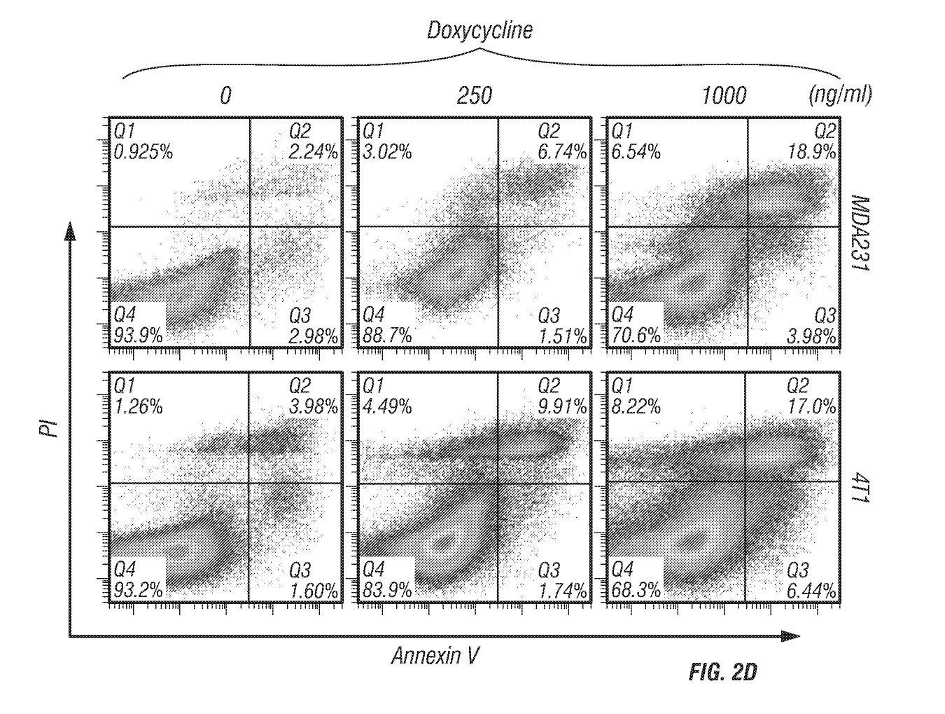

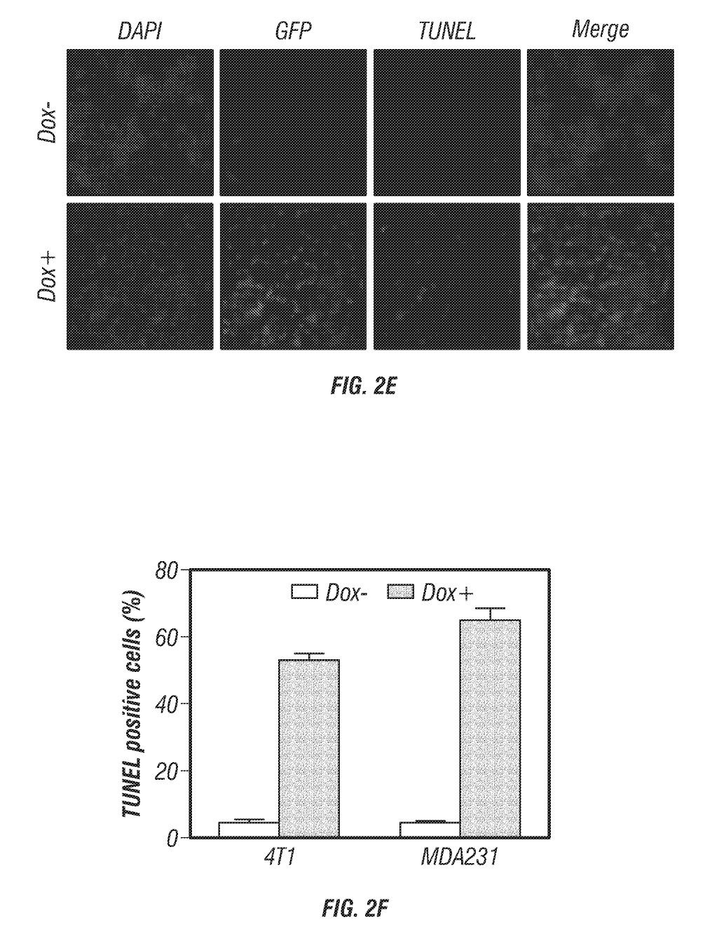

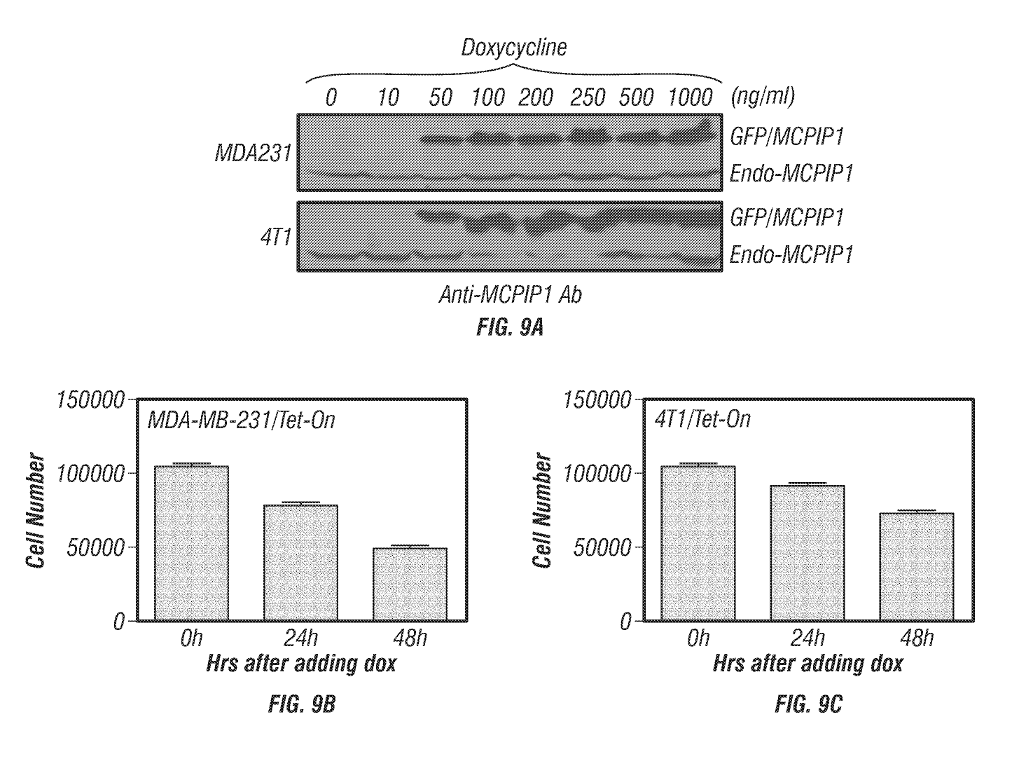

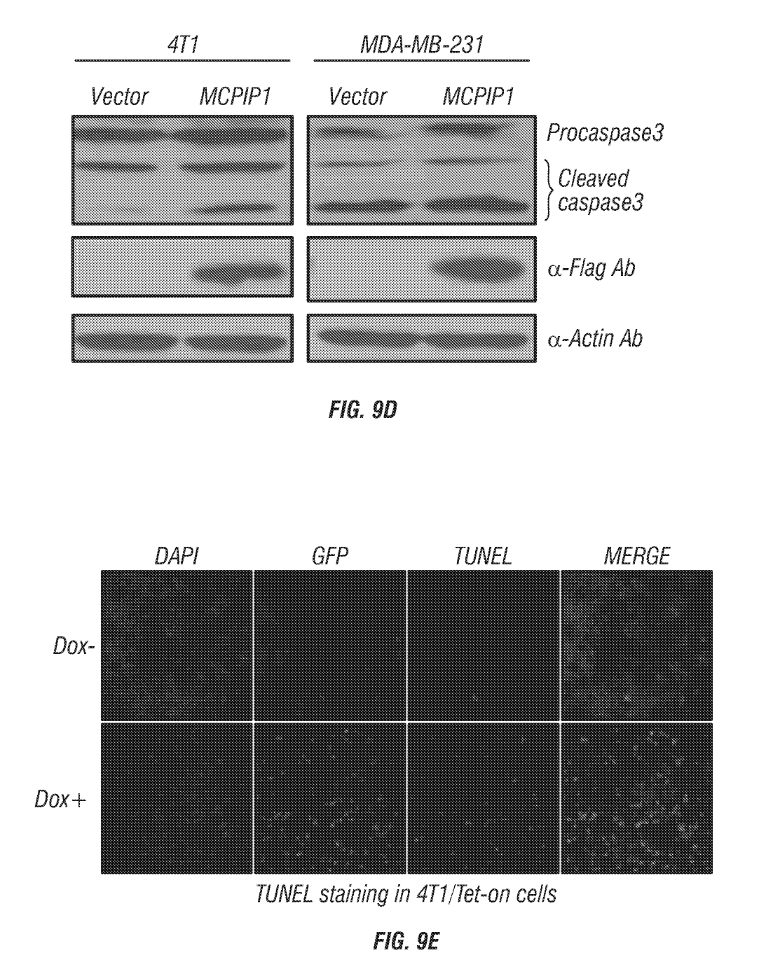

To study MCPIP1 function, the inventors the inventors generated inducible expression of MCPIP1 in human MDA-MB-231 and mouse 4T1 breast tumor cells with the Tet-on system. Addition of Doxycycline (Dox) dose-dependently induced GFP/MCPIP1 fusion protein in both cells (FIGS. 2A and 9A). When MCPIP1 was induced, the inventors noticed a loss of cell number of both tumor cells (FIGS. 9B-C). To determine whether MCPIP1 induced cell death by apoptosis, the inventors measured the expression of two apoptosis markers, Caspase3 and PARP1, in cells expressing MCPIP1. MCPIP1, in a time-dependent and dose-dependent manner, caused cleavages of Caspase3 (FIG. 2B) and PARP1 (FIG. 2C) in MDA-MB-231 cells (upper panel) and 4T1 cells (lower panel). Transient expression of flag-tagged MCPIP1 in 4T1 and MDA-MB-231 cells also induced cleavages of Capase3 (FIG. 9D). In addition, MCPIP1 caused an increase in PI+ and Annexin V+ apoptotic MDA-MB-231 and 4T1 tumor cells in a dose-dependent manner (FIG. 2D). By Tunel staining, the inventors found more Tunel+ apoptotic MDA-MB-231 cells (FIGS. 2E-F) and 4T1 cells (FIG. 9E) after MCPIP1 induced by Dox. These data clearly demonstrate that MCPIP1 induces apoptosis of breast tumor cells.

MCPIP1 Targets Anti-Apoptotic Genes for Apoptosis.

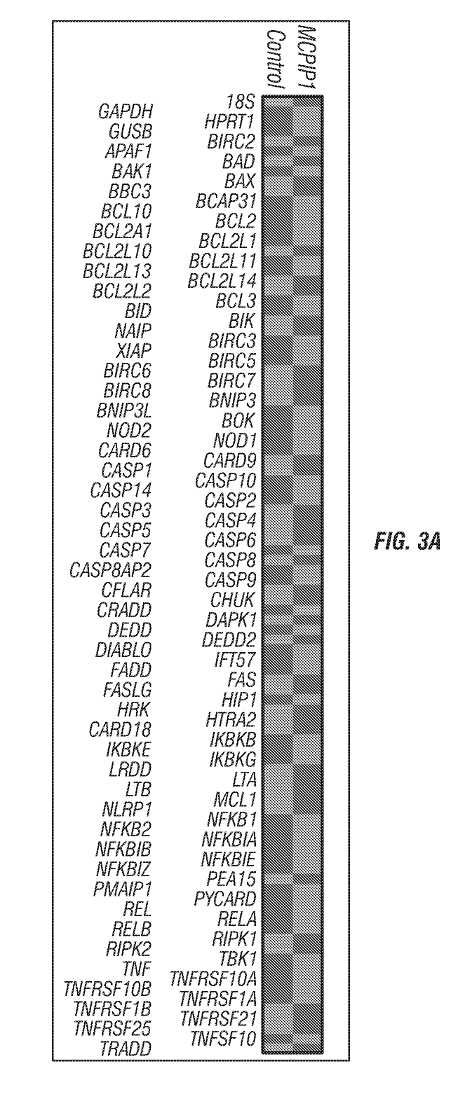

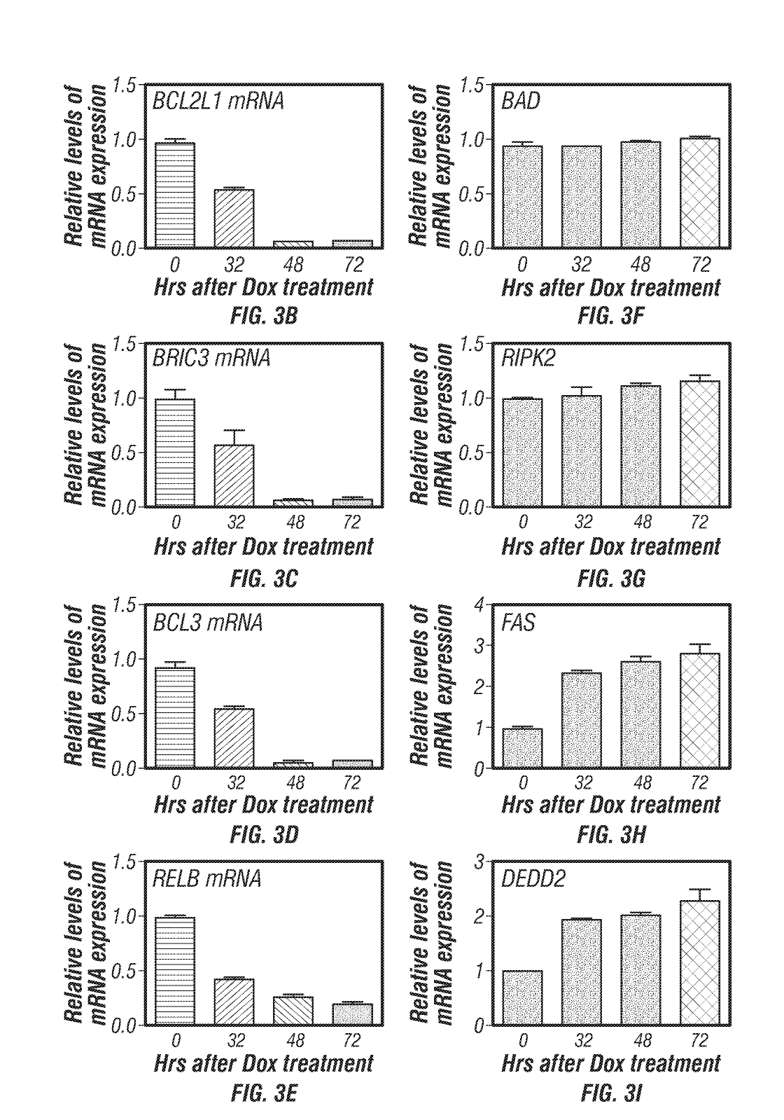

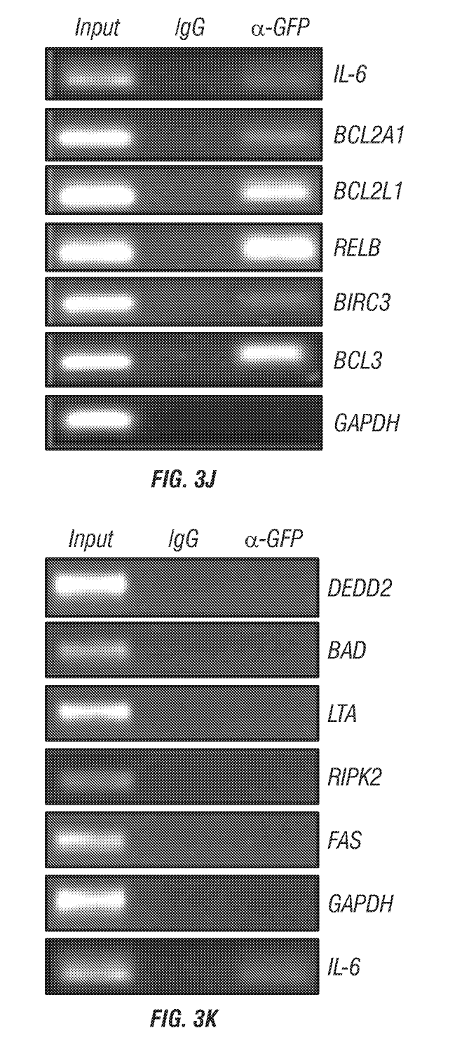

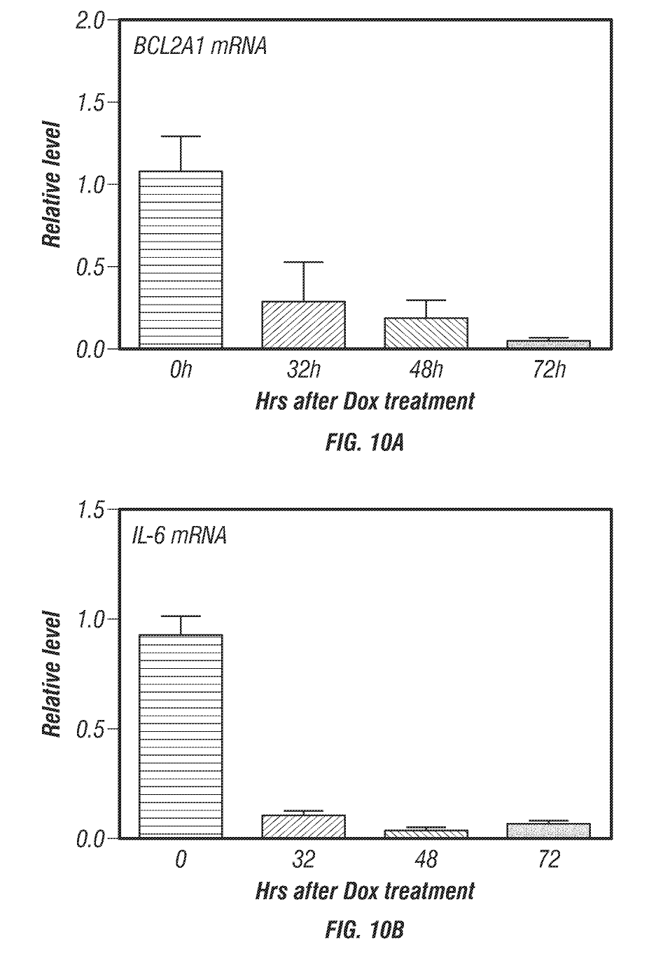

The inventors next identified the genes in apoptosis pathway affected by MCPIP1 using the TaqMan® Human Apoptosis PCR Array kit (Life Technologies). In MCPIP1-expressing MDA-MB-231 cells, they identified 31 transcripts affected by MCPIP1 expression (FIG. 3A), with 6 anti-apoptotic genes down-regulated and 25 pro-apoptotic genes up-regulated (Table S1). To validate the array results, the inventors measured the mRNAs of five down-regulated anti-apoptotic genes (Bcl2L1. Bcl2A1. Birc3. RelB, and Bcl3) and four up-regulated pro-apoptotic genes (Bad. Ripk2. Fas. and Dedd2) by real-time PCR. The expressions of five anti-apoptotic gene transcripts were reduced in a time-dependent manner in cells expressed MCPIP1 (FIGS. 3B-E and 10A). In contrast, no changes in pro-apoptotic BAD (FIG. 3F) and RIPK2 (FIG. 3G) transcripts and minor increase in FAS (FIG. 3H) and DEDD2 (FIG. 3I) transcripts were observed upon MCPIP1 induction. To determine whether MCPIP1 bound to these anti-apoptotic transcripts, the inventors first induced GFP/MCPIP1 fusion protein in MDA-MB-231/Tet-On cells with Dox, and then pull-downed the MCPIP1-binding complex with anti-GFP antibody from cytoplasmic extracts (Keene et al., 2006), followed by detecting the bound mRNAs by RT-PCR. All five anti-apoptotic gene mRNAs were amplified by PCR but not the GAPDH mRNA (FIG. 3J) and the pro-apoptotic mRNAs (FIG. 3K). IL-6 was served as positive control (FIG. 10B). These data indicate that MCPIP1 preferentially inhibits the mRNA expression of anti-apoptotic genes for inducing apoptosis in breast tumor cells.

MCPIP1 Targets the mRNA of Anti-Apoptotic Genes for Degradation Via the PIN Domain.

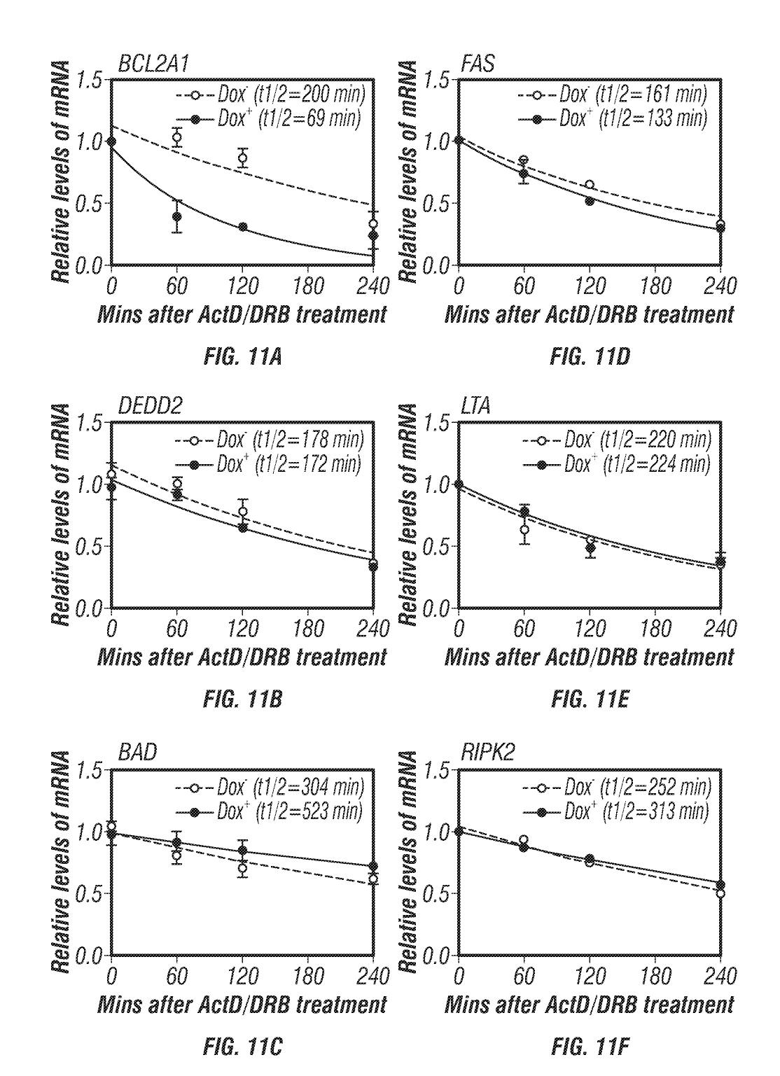

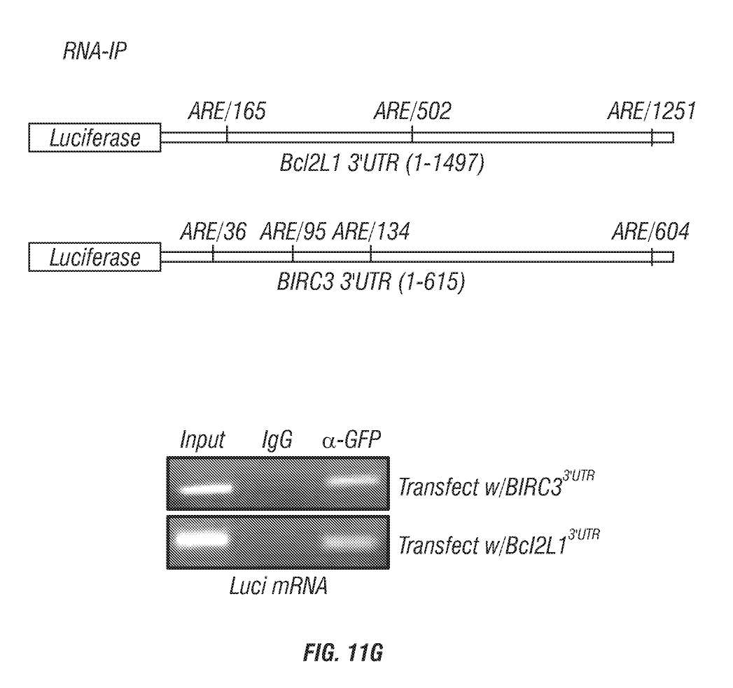

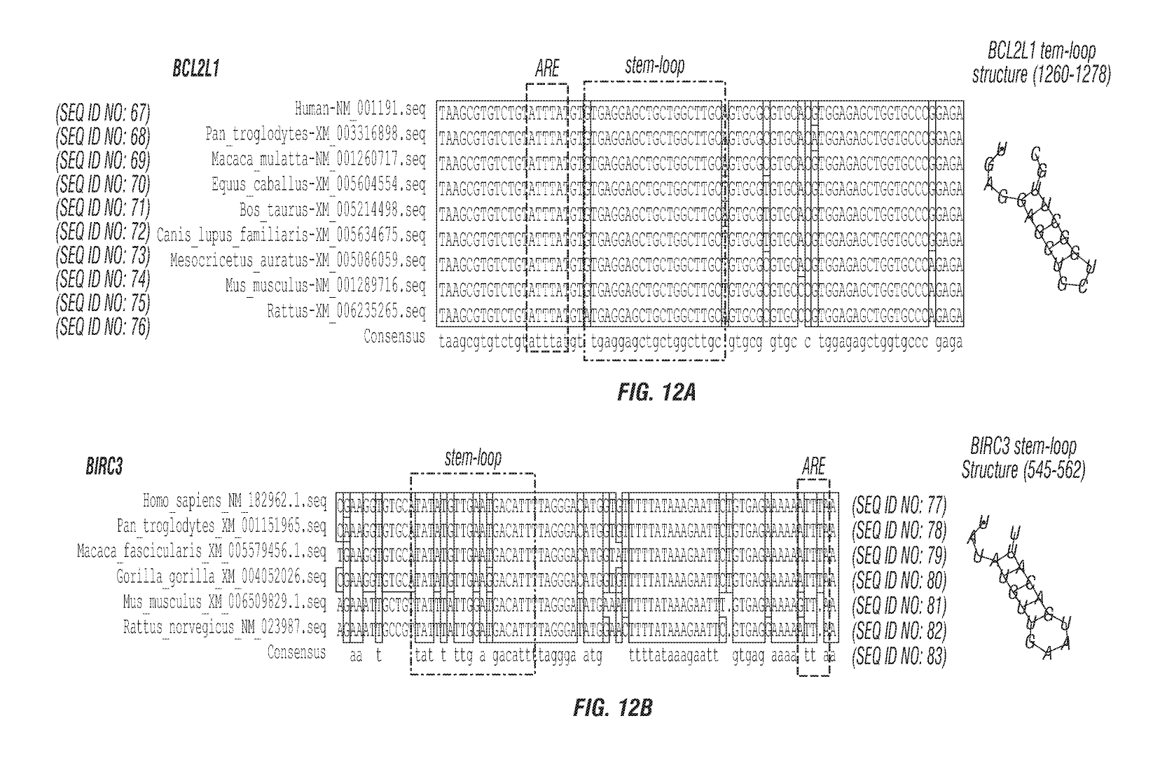

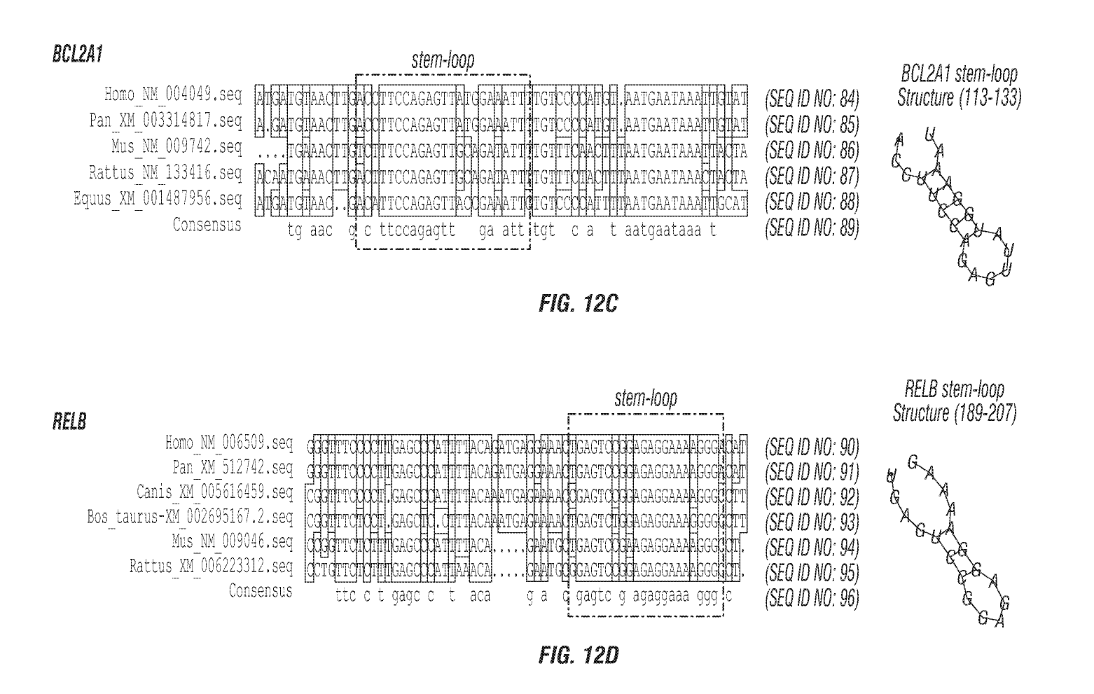

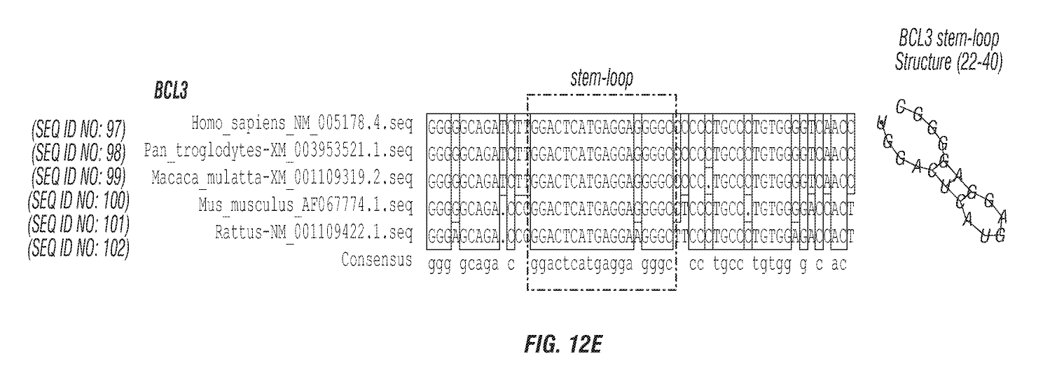

Since MCPIP1 inhibits the mRNA expression of anti-apoptotic genes, the inventors speculated that MCPIP1 might trigger apoptosis through destabilizing the mRNAs of these anti-apoptotic genes. The inventors first induced MCPIP1 expression and then blocked de novo synthesis of mRNA with Actinomycin D and DRB, followed by measuring the remaining mRNAs at different time points. The half-lives of anti-apoptotic mRNAs, including Bcl2L1 (FIG. 4A). Bcl3 (FIG. 4B). BIRC3 (FIG. 4C). RelB (FIG. 4D), and Bcl2A1 (FIG. 11A), were shorten about two folds in MCPIP1-expressing cells (Dox+) compared to cells without MCPIP1 induction (Dox−), whereas MCPIP1 expression had little effects on half-lives of the mRNAs encoding pro-apoptotic DEDD2, BAD, FAS, LTA, and RIPK2 (FIGS. 11B-F), demonstrating that MCPIP1 induces apoptosis through directly targeting mRNAs of the anti-apoptosis genes but not pro-apoptosis gene for degradation.

To determine if the mRNA decay was mediated through 3′UTR, the inventors cloned the 3′UTRs of Bcl2L1. RelB. BIRC3 and Bcl3 downstream of the luciferase gene as previously described (Qian et al., 2011), and then co-transfected the 3′UTR reporter constructs with MCPIP1 expression plasmid into HEK293 cells, followed by measuring luciferase activity. As shown in FIG. 4E, MCPIP1 suppressed luciferase activities of all four 3′UTR reporter constructs compared to cells transfected with control vector. Since β-actin mRNA is quite stable and not affected by its 3′UTR, MCPIP1 did not affect its luciferase activity. Luciferase mRNA linked to the 3′UTRs of Bcl2L1 and BRIC3 was also pulled down by GFP antibody (FIG. 11G). These results indicate that MCPIP1 acts through the 3′UTR to decay anti-apoptotic gene transcripts.

MCPIP1 protein contains a PIN-like domain which is reported to have an RNase activity and a zinc finger domain thought to interact with AU-rich elements (ARE) located in the 3′UTR of mRNAs (Matsushita et al., 2009 and Liang et al., 2010). To determine which domains in MCPIP1 are responsible for mRNA decay, the inventors mutated the PIN domain (D141N) and the ZF domain (ΔZF) as illustrated in FIG. 4F, and respectively co-transfected the mutants as well as wild-type MCPCP1 with different 3′UTR reporter constructs into HEk293 cells. Wild-type and ZF mutant MCPIP1 suppressed the luciferase activities of all four 3′UTR reporter constructs (FIG. 4G) and their mRNA expression (FIG. 4H), while D141N mutant MCPIP1 completely loosed its suppressive effects. In addition, MDA-MB-231 cells transfected with the D141N mutant MCPIP1 did not cause cell death (FIG. 4I) and failed to cleave the PARP1 (FIG. 4J). These results demonstrate that the PIN domain in MCPIP1 is responsible for mRNA decay of the anti-apoptotic genes and for apoptosis induction.

MCPIP1 Depletion Stabilizes Anti-Apoptotic Gene Transcripts and Promotes Tumor Cell Proliferation.

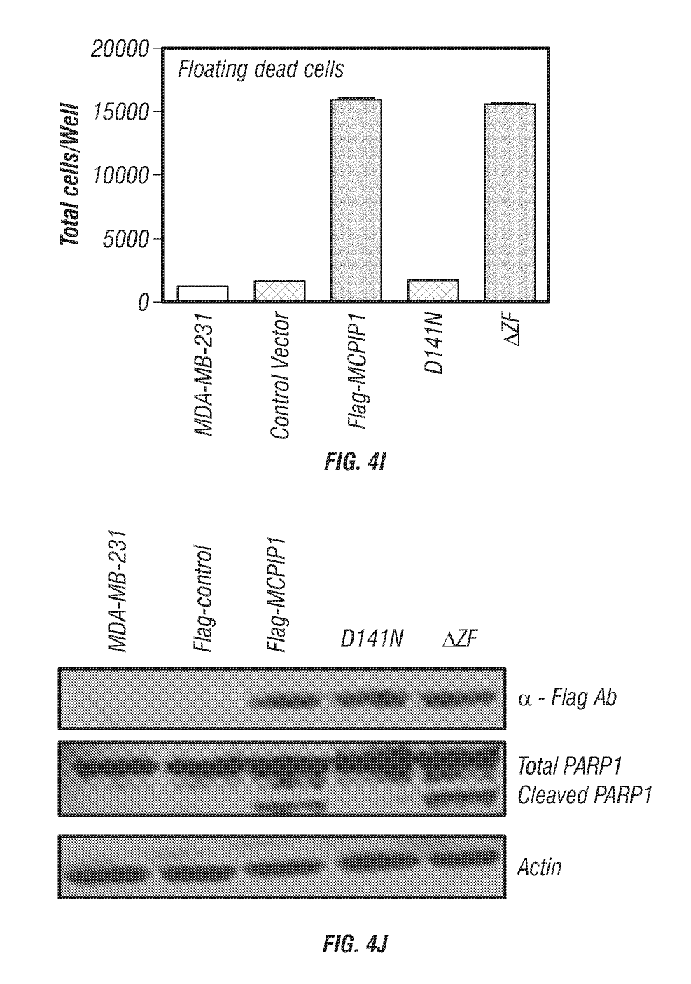

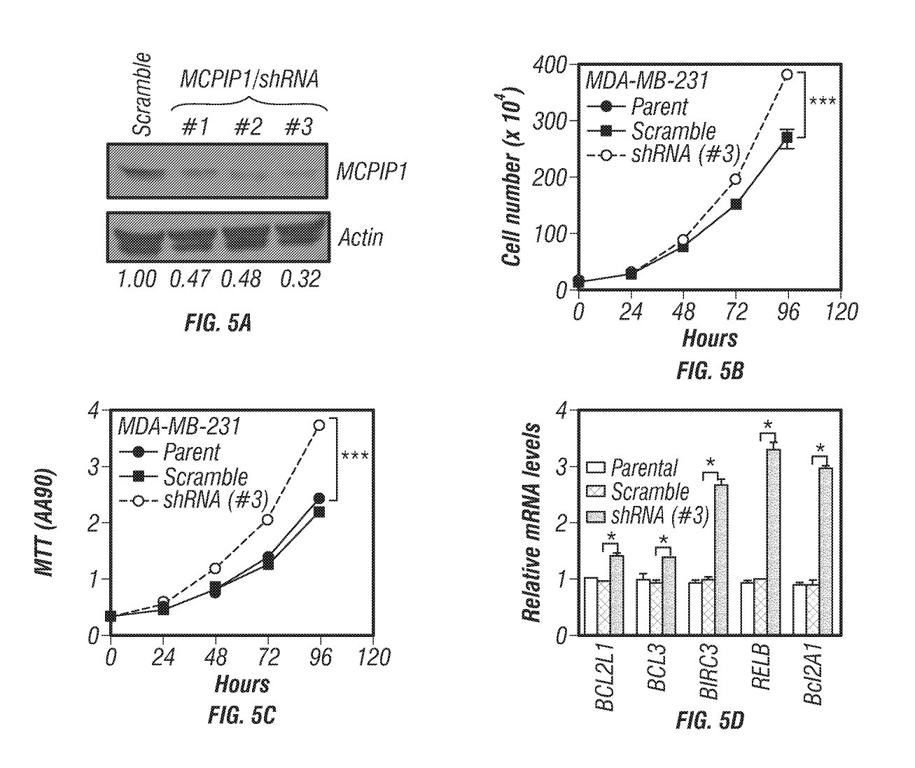

To confirm the inductive effects of MCPIP1 on tumor apoptosis, the inventors further reduced MCPIP1 expression with shRNA in MDA-MB-231 cells. The MCPIP1 protein was knocked-down about 53%-68% by three shRNAs, respectively, with the 3rd shRNA having the highest knockdown efficiency (˜68%) compared with scramble control (FIG. 5A). Therefore, the inventors used the 3rd shRNA/MCPIP1 (#3) for subsequent study. Though MCPIP1 expression is repressed in tumor cells, further knocking-down MCPIP1 by shRNA significantly enhanced tumor cell proliferation as indicated by cell counting (FIG. 5B) and MTT assay (FIG. 5C). In agreement with the overexpression results, knocking-down MCPIP1 expression further increased the mRNA expression of anti-apoptotic genes, including bcl211, bcl3, birc3, relb and bcl2a1 (FIG. 5D), while the mRNA of pro-apoptotic genes (bad, ripk2, fas and dedd2) did not change between MCPIP1 knocking-down and scramble cells (data not shown). Since MCPIP1 overexpression enhanced mRNA degradation of the anti-apoptosis genes, the inventors wondered whether deletion of MCPIP1 could stabilize the mRNAs of the anti-apoptotic genes. Indeed, the half-lives of four MCPIP1 targets were increased in MDA-MB0231 cells after knocking-down MCPIP1 with shRNA/MCPIP1 lentivirus, with the half-life of Bcl211 mRNA being increased from 191 min to 375 min (FIG. 5E), Bcl3 from 170 min to 202 min (FIG. 5F), BIRC3 from 133 min to 187 min (FIG. 5G), and RELB from 355 min to 428 min (FIG. 5H), respectively. Furthermore, the inventors also observed a similar pattern of increase in cell proliferation of MDA-MB-453 cells after knocking-down MCPIP1 (data not shown). These results confirm that MCPIP1 repression indeed promotes breast cancer cell proliferation and induces tumor cell apoptosis-resistance through stabilizing anti-apoptotic transcripts.

MCPIP1 Binds to a Stem-Loop Structure in the 3′UTR of Anti-Apoptotic mRNAs for Degradation.

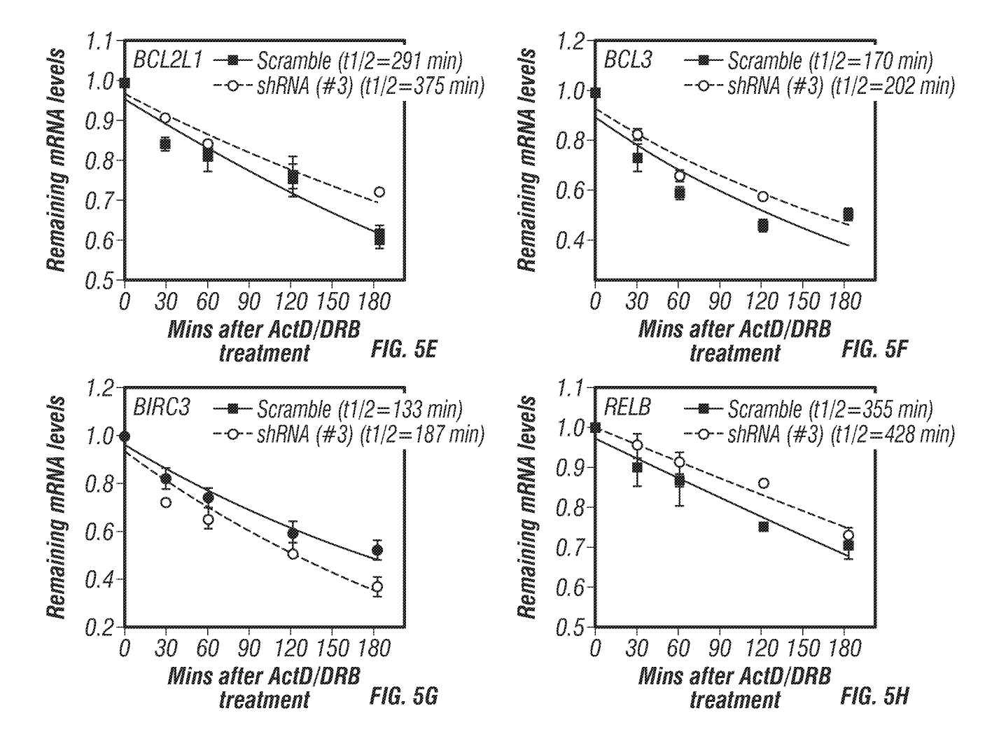

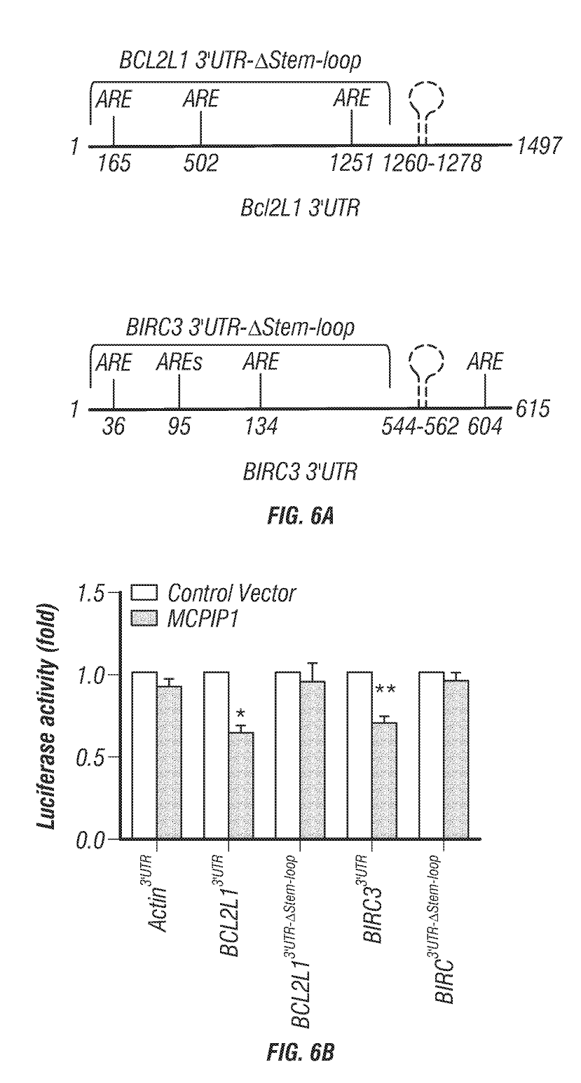

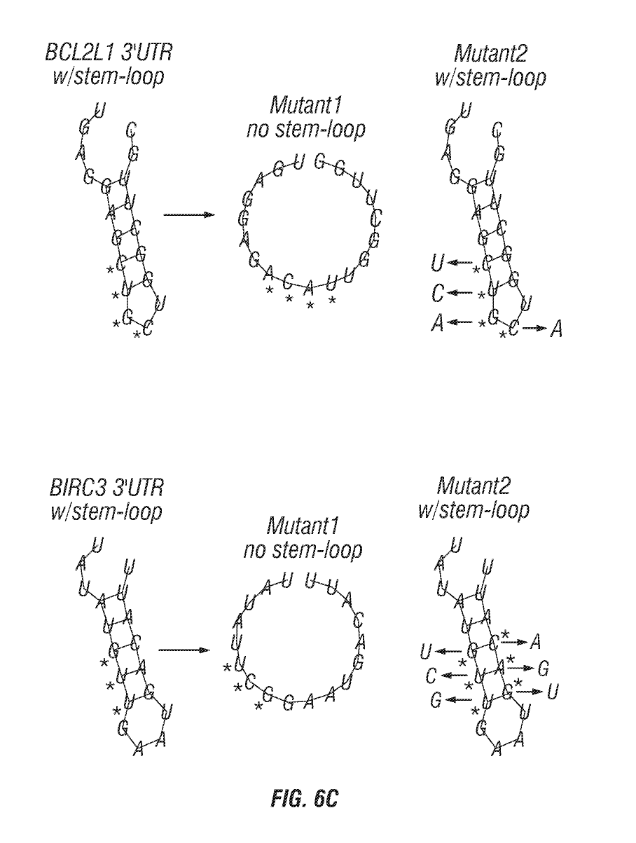

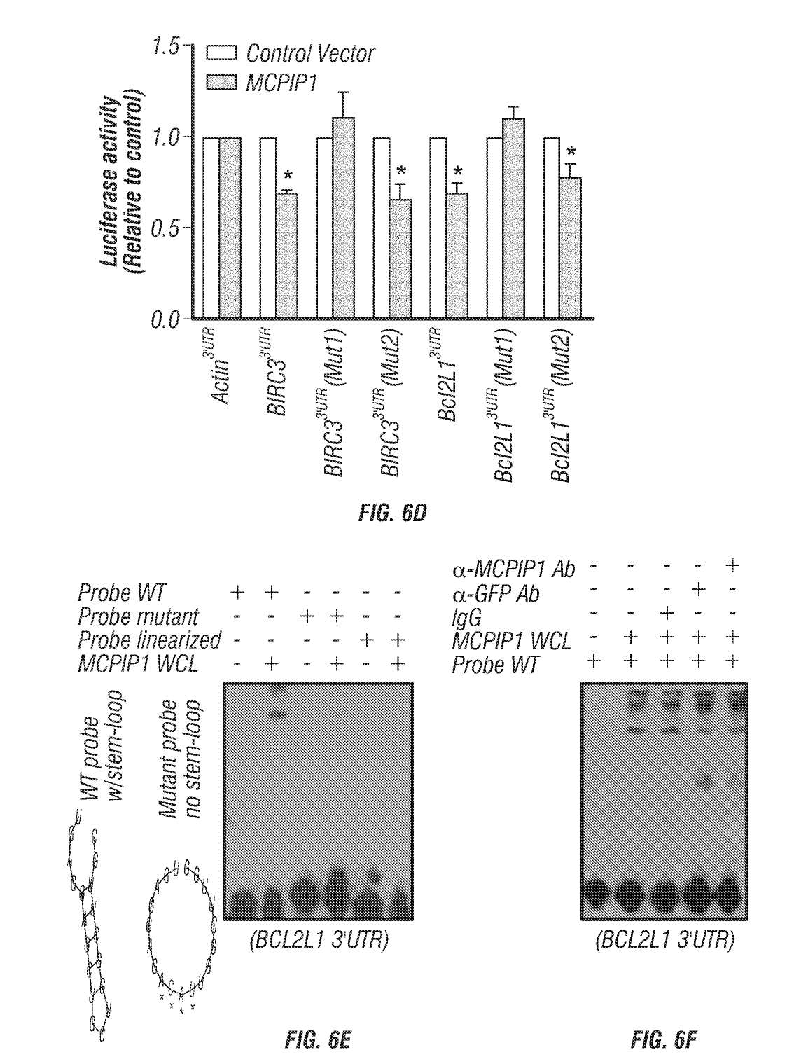

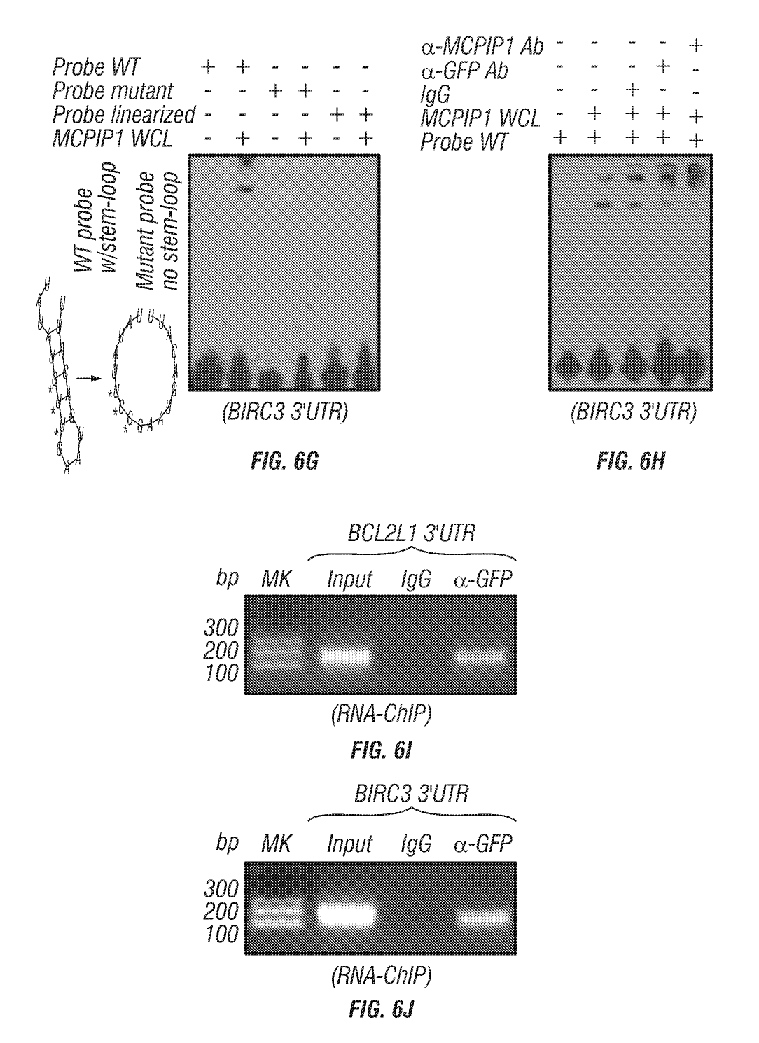

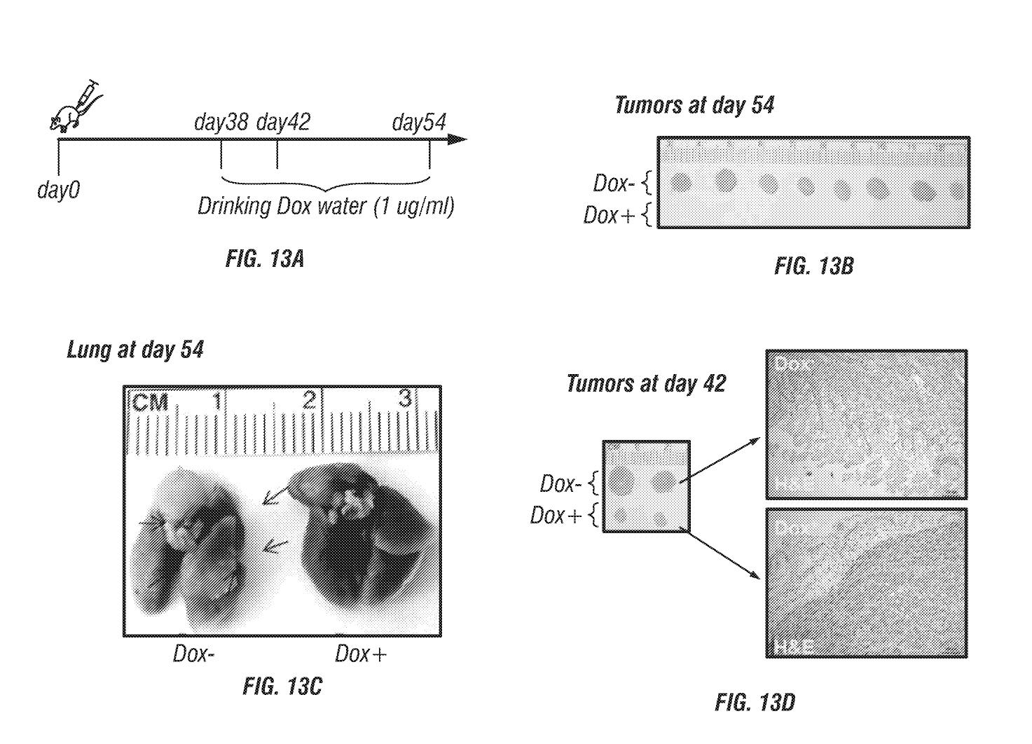

MCPIP1 is known to decay IL6 and IL2 mRNAs through stem-loop structure (Iwasaki et al., 2011 and Li et al., 2012). The inventors compared the 3′UTR sequences of five anti-apoptotic genes among different species and identified a conserved sequence. Interestingly, a stem-loop structure could be formed within the conserved sequences when analyzed the sequences with RNAfold (Hofacker, 2003) (FIGS. 13A-E). To define the role of stem-loop structure in MCPIP1-mediated mRNA decay, the inventors generated deletion constructs by deleting the sequences containing the stem-loop in the 3′UTRs of Bcl2L1 and BIRC3, and then co-transfected wild-type and deletion reporters with MCPIP1 into HEK293 cells, followed by luciferase measurement. MCPIP1 suppressed the luciferase activities in cells transfected with full-length BCL2L1 and BIRC3 3′UTR constructs, but had no effects on cells transfected with the deletion constructs (FIG. 6B), suggesting the requirement for stem-loop structure but not ARE for mRNA decay. To confirm the dependence of MCPIP1-induced mRNA decay on stem-loop structure but not absolute nucleotide, the inventors generated two 3′UTR mutation constructs of BIRC3 and BCL2L1, mutant1 with the step-loop being disrupted by substitution of four nucleotides and mutant2 still kept the stem-loop though substitution of four different nucleotides in stem and loop region as illustrated in FIG. 6C, and then co-transfected the two mutants with MCPIP1 for measuring luciferase activity. Disruption of the stem-loop structure in the 3′UTRs of BIRC3 and Bcl2L1 (mutant1) made them completely resistant to MCPIP1 inhibition, whereas the mutant kept the intact step-loop structure (mutant2) were still susceptible to MCPIP1 suppression (FIG. 6D), further confirming that MCPIP1 destabilizes the mRNAs of anti-apoptotic genes through the stem-loop structure in their 3′UTRs independent of the absolute nucleotide sequence.

To determine if MCPIP1 physically bound to the stem-loop structure in the 3′UTRs of Bcl2L1 and BIRC3, the inventors performed RNA-EMSA with wild-type RNA probes harboring the step-loop structure, and RNA probes with the step-loop structure either disrupted by nucleotide substitution (mutant probe) or eliminated by physical force (linearized probe) according to manufacturer's manual (Fisher). There was a unique RNA-cytoplasmic protein binding complex formed only to the WT probe but not to the mutant and linearized probes of Bcl2L1 (FIG. 6E) and BIRC3 (FIG. 6G). To confirm MCPIP1 binding, the inventors added anti-GFP and anti-MCPIP1 antibodies to the cytoplasmic proteins and performed supershift RNA-EMSA. The binding intensity was significantly reduced by adding either anti-GFP antibody or anti-MCPIP1 antibody but not control IgG (FIGS. 6F and 6H), indicating that MCPIP1 indeed physically binds to the stem-loop structures in the 3′UTRs of Bcl2L1 and BIRC3. Furthermore, the inventors performed a modified RNA immunoprecipitation-chromatin immunoprecipitation assay (RIP-ChIP) (Keene et al., 2006) to determine whether MCPIP1 bound to the 3′UTRs of anti-apoptotic genes in vivo. After induced MCPIP1/GFP fusion protein in MDA-MB-231 cells, the inventors precipitated the complex with anti-GFP antibody and fragmented the binding mRNAs, followed by amplifying the stem-loop sequence by PCR. The stem-loop sequences in the 3′UTRs of Bcl2L1 (FIG. 61) and BIRC3 (FIG. 6J) were amplified in groups pulled down with anti-GFP antibody but not the IgG, indicating the binding of MCPIP1 to the 3′UTRs of these anti-apoptotic mRNAs inside breast tumor cells. Taken together, these data demonstrate that MCPIP1 recognizes the stem-loop structure in the 3′UTRs of anti-apoptotic gene transcripts for degradation and apoptosis induction.

MCPIP1 Suppresses Breast Tumor Growth and Metastasis.

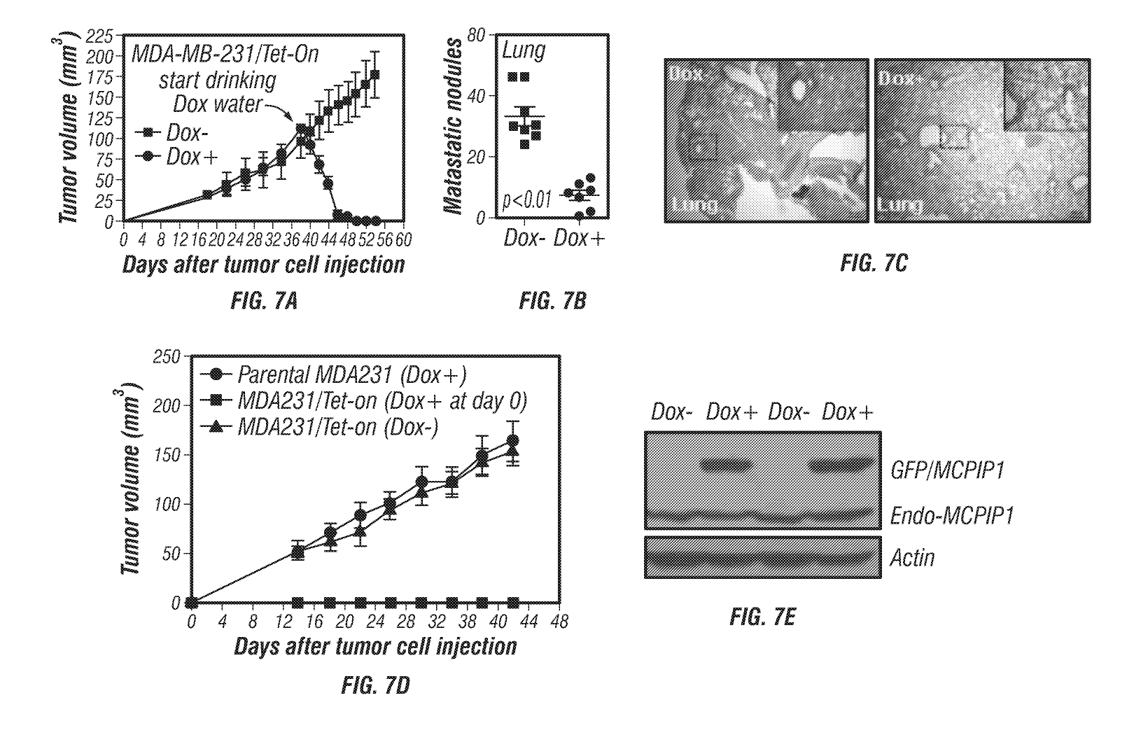

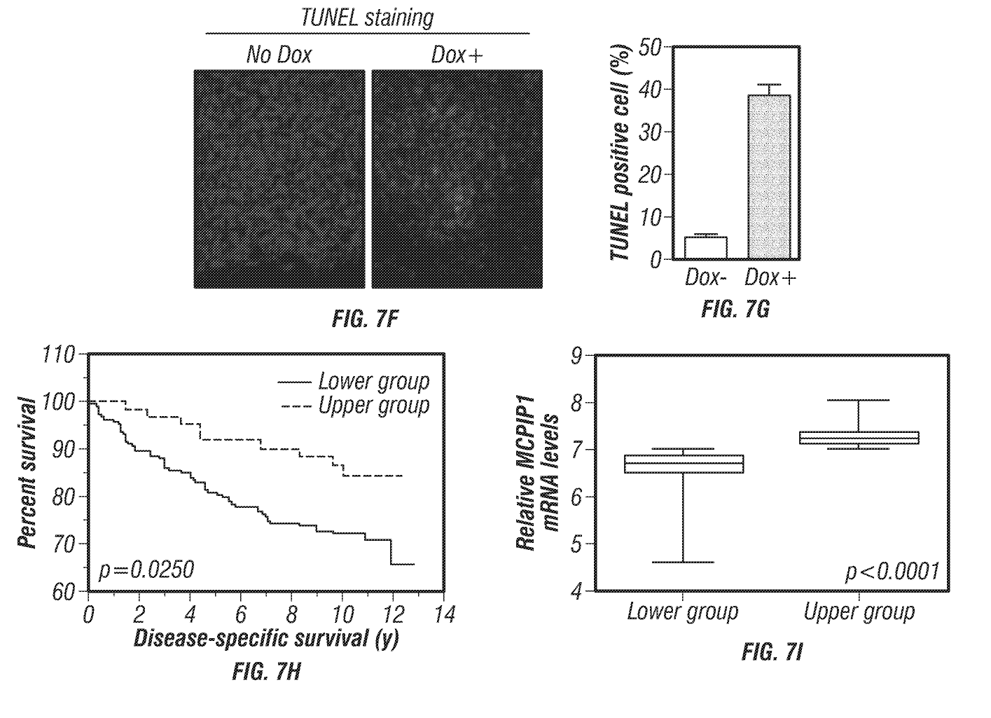

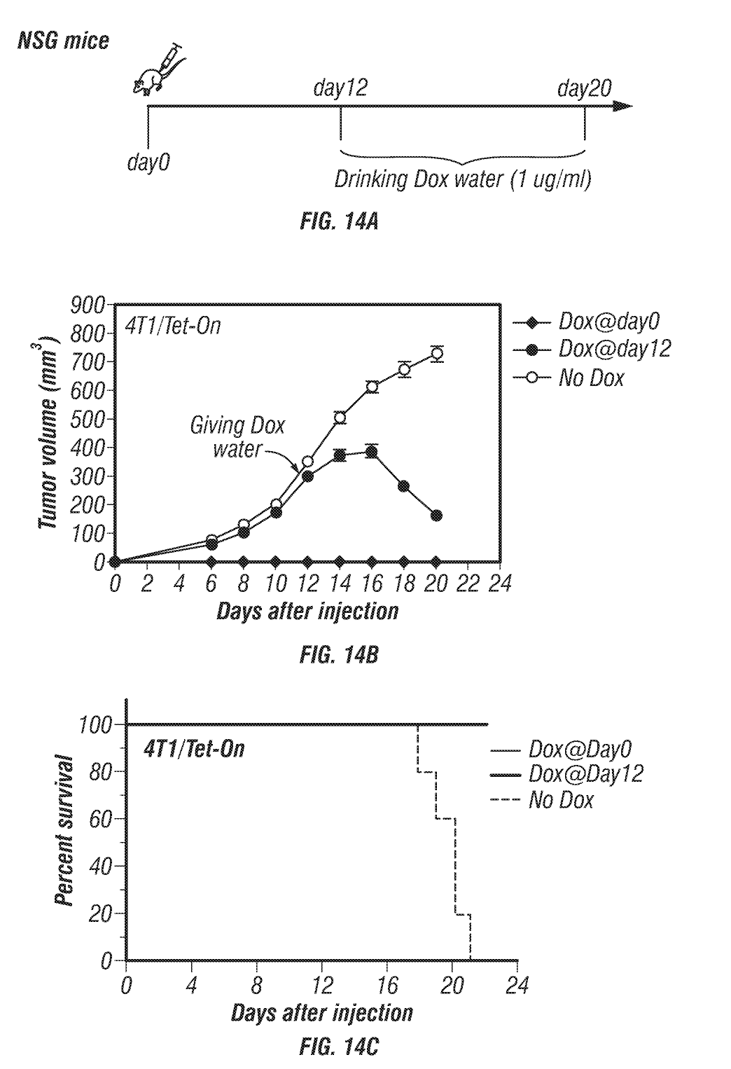

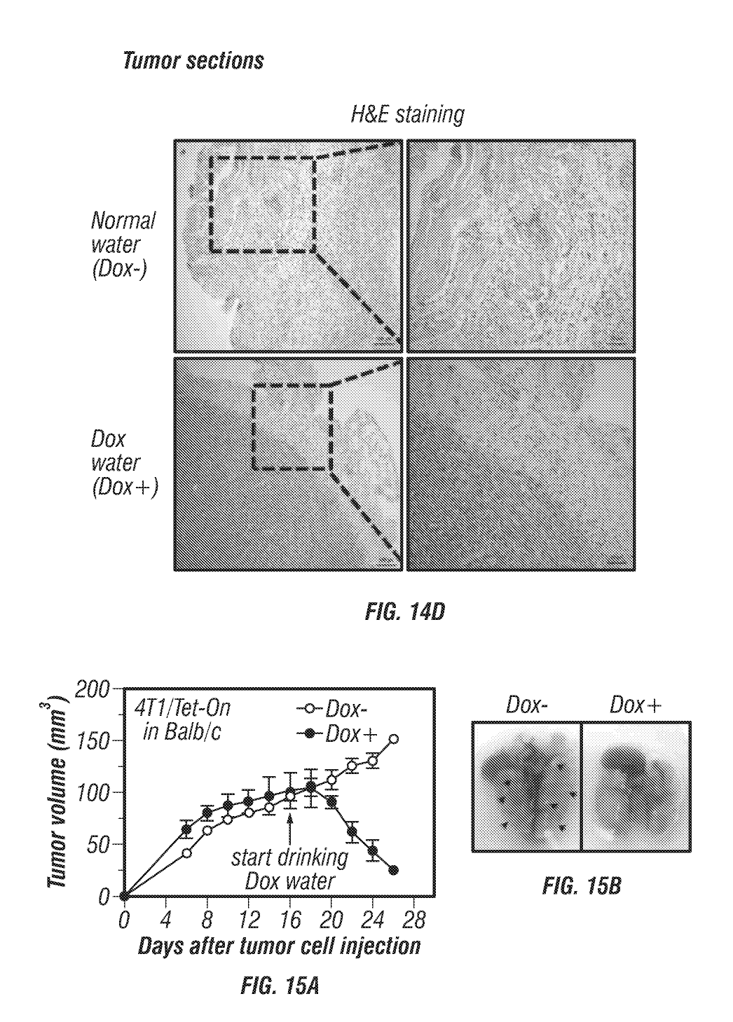

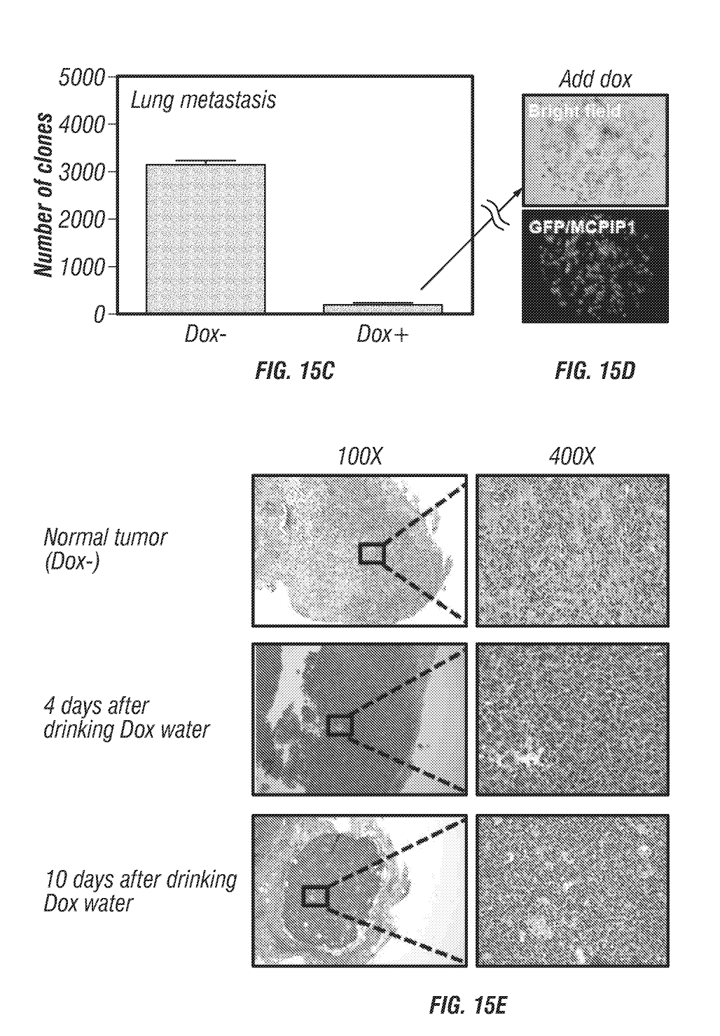

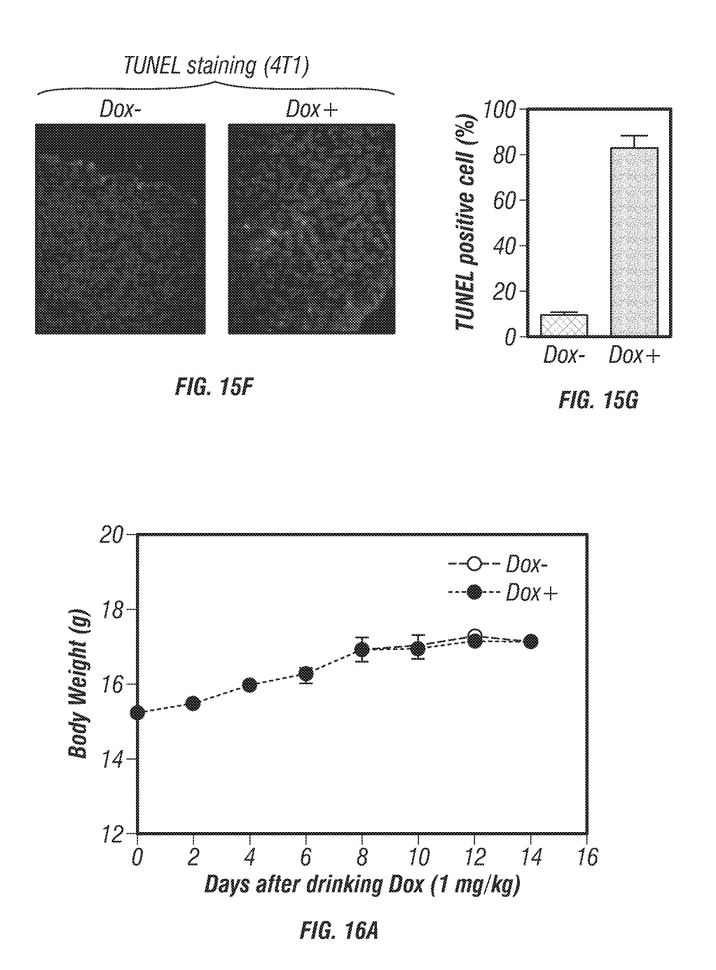

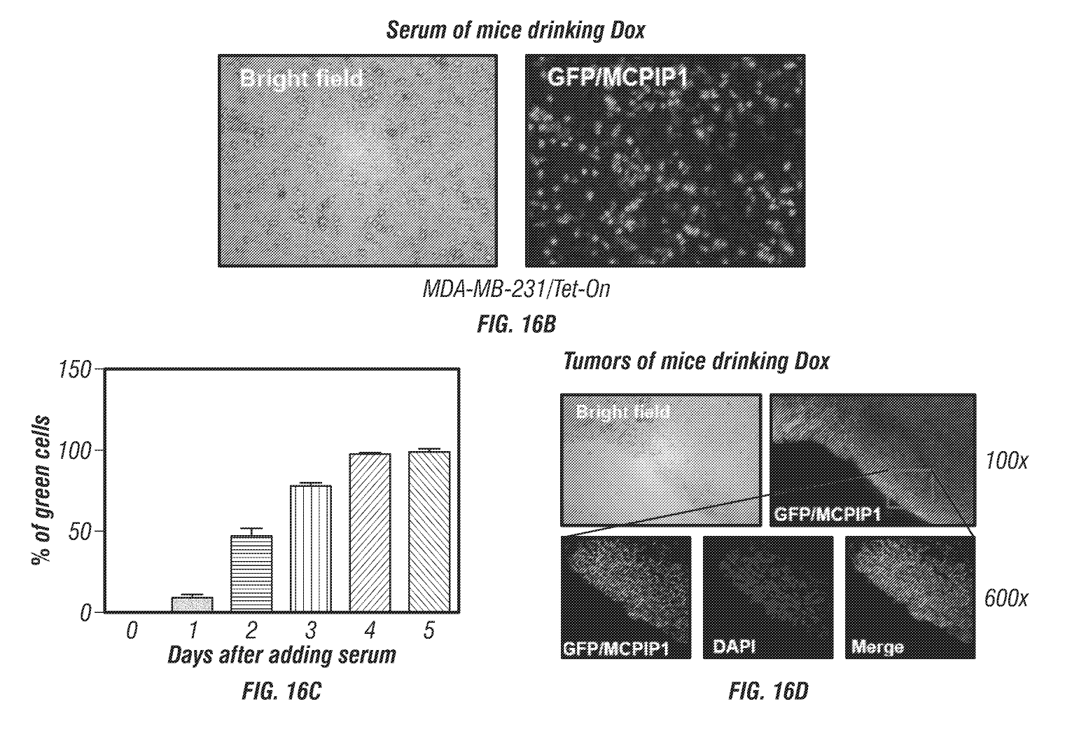

To determine if MCPIP1 suppressed breast tumor progression, the inventors inoculated the MDA-MB-231/Tet-On tumor cells into mammary glands of immune-compromised NSG mice. When tumor mass reaching 5 mm in diameter, the inventors induced MCPIP1 by feeding the tumor-bearing mice with Dox-containing water (Dox+) (FIG. 14A). One day after drinking Dox water, tumors started to shrink and then rapidly disappeared in six days, while tumors in mice fed with normal water (Dox−) kept growing (FIGS. 7A and 14B). 54 days after tumor cell inoculation, the inventors euthanized the mice, counted metastatic lung nodules, and compared it between mice with and without Dox water. There was a significant reduction in lung metastases in mice drinking Dox water compared to mice fed with normal water (FIGS. 7B and 14C). Histological analysis also showed much less micro-metastatic sites in the lungs of Dox-feeding mice compared to mice without Dox water (FIG. 7C). When Dox water was fed at the same time as tumor cell inoculation (day 0), no tumors were formed; again tumors kept growth in mice fed with normal water (Dox−) (FIG. 7D). Meanwhile, Dox-containing water had no effects on tumor growth in mice inoculated with parental tumor cells (FIG. 7D), indicating specific anti-tumor effects of MCPIP1. Indeed, MCPIP1 was highly induced in tumors of mice fed with dox-water (FIG. 7E). To determine whether tumor suppression was due to apoptosis, the inventors performed TUNEL staining and results showed that apoptotic cells were significantly increased in tumors of mice drinking dox-water compared with mice fed with normal water (FIGS. 7F-G). To further confirm broader anti-tumor effects of MCPIP1 in vivo, the inventors inoculated the 4T1/Tet-on tumor cells into mammary glands of immunocompromised NSG mice (FIGS. 14A-D) and into mammary glands of immunocompetent Balb/c mice (FIGS. 15A-G). Similar to MDA-MB-231 cells, a strong suppression on tumor growth and metastases by MCPIP1 were observed in both models (FIGS. 14-15). Taken together, these results demonstrate that MCPIP1 is a potent tumor suppressor that strongly suppresses breast tumor growth and metastasis by activation of apoptosis.

To determine if MCPIP1 was associated with tumor aggressiveness in breast cancer patients, the inventors surveyed MCPIP1 expression across a gene array data set derived from the excised breast tumors of 251 patients (Miller et al., 2005). Using this cohort, the inventors analyzed breast cancer-specific survival of these 251 patients between high and low MCPIP1 expression groups. As shown in FIG. 7H, patients with low tumor MCPIP1 mRNA levels were more likely to die from recurrent breast cancer following tumorectomy than patients whose tumors expressed high levels of MCPIP1 at excision. The difference in MCPIP1 expression between two groups was statistically significant (FIG. 71). These results indicate that MCPIP1 is a new biomarker for prognosis of breast cancer patients and also confirm that MCPIP1 is a new tumor suppressor of breast cancer.

MCPIP1 Suppresses Tumor Cell Growth in General.

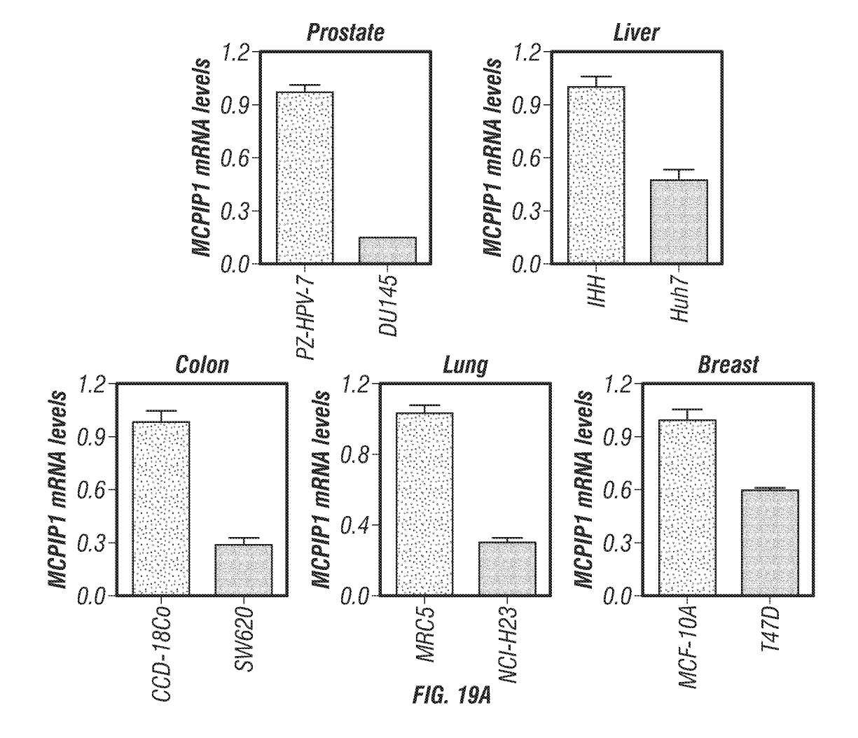

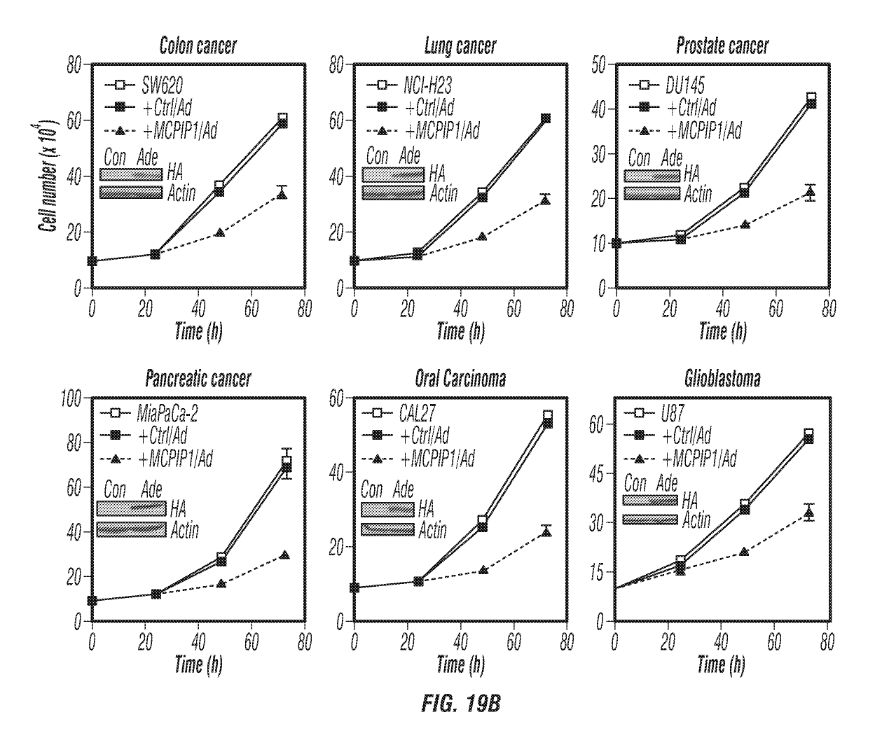

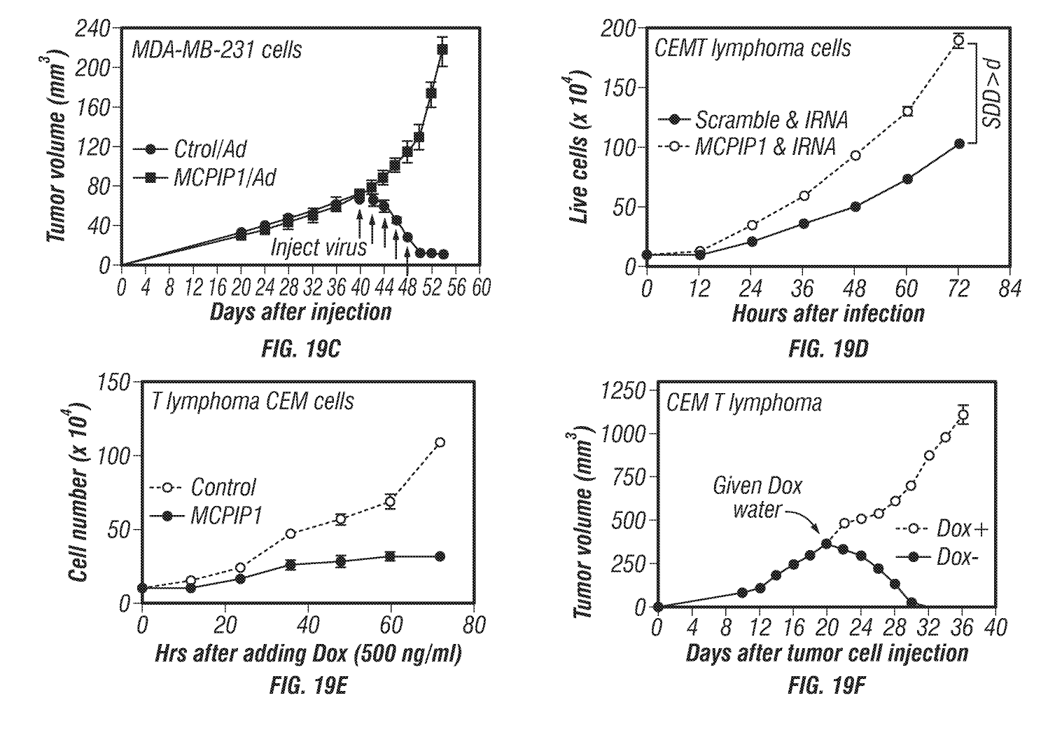

To further determine whether MCPIP1 is a general tumor suppressor, we tested the levels of MCPIP1 in various types of tumors and found that the expression of MCPIP1 mRNA (FIG. 19A) and protein (data not shown) were significantly reduced in tumor cells compared to their respect normal cells. Then we infected these tumor cells by MCPIP1-expressing adenovirus or control empty adenovirus, followed by measuring cell survival. Expressing MCPIP1 by adenovirus significantly suppressed tumor cell growth (FIG. 19B), including cancer cells from colon, lung, prostate, pancreatic, oral carcinoma, glioblastoma, and breast, indicating that MCPIP1 is a potent tumor suppressor that can inhibit multiple tumor growth. In addition, to determine the effects of MCPIP1/adenovirus on tumor growth in vivo, we induced MDA-MB-231 tumors in the NSG mice and injected the MCPIP1/Ad into tumors when tumor reached certain size. As shown in FIG. 19C, MCPIP1/Ad almost completely eliminated the established tumors, further indicating the potential of using MCPIP1/Ad or other means to express MCPIP1 for tumor treatments. Moreover, we tested the effects of MCPIP1 on liquid tumors, such as lymphoma. Deletion of MCPIP1 in T lymphoma cells (CEM-ss) increased cell proliferation (FIG. 19D), while over-expressing MCPIP1 by adding Dox suppressed CEM cell growth (FIG. 19E), indicating that MCPIP1 can also inhibit growth of T cell lymphoma. Indeed, inducing MCPIP1 by feeding the mice harboring the CEM-tumors almost completely regressed the lymphoma (FIG. 19F). Taken together, these results indicate that MCPIP1 is a general tumor suppressor, and expressing or inducing MCPIP1 can be used to treat various tumors, including solid and liquid tumors.

TABLE S1
List of Apoptosis-Related Genes Affected by MCPIP1

| Gene | | | | Fold |
|---|---|---|---|---|
| Symbol | Gene Name | Control | MCPIP1 | Change |
| 18S | Eukaryotic 18S rRNA | 15.99170685 | 14.98009396 | NA |
| GAPDH | glyceraldehyde-3-phosphate dehydrogenase | 16.97334671 | 17.96116066 | NA |
| HPRT1 | hypoxanthine phosphoribosyltransferase 1 | 21.95671082 | 22.93836212 | NA |
| | (Lesch-Nyhan syndrome) | | | |
| GUSB | glucuronidase, beta | 23.96611595 | 24.97103119 | −0.012 |
| BIRC2 | baculoviral IAP repeat-containing 2 | 26.98887062 | 27.02367592 | 0.936 |
| APAF1 | apoptotic peptidase activating factor 1 | 28.97918129 | 29.97677422 | −0.007 |
| BAD | BCL2-antagonist of cell death | 35.97512054 | 35.98209 | 0.993 |
| BAK1 | BCL2-antagonist/killer 1 | 24.97545433 | 25.97936249 | −0.011 |
| BAX | BCL2-associated X protein | 24.98869133 | 24.97661972 | 1 |
| BBC3 | BCL2 binding component 3 | 34.97323608 | 34.9589386 | 1.003 |
| BCAP31 | B-cell receptor-associated protein 31 | 20.97003555 | 21.9615345 | −0.003 |
| BCL10 | B-cell CLL/lymphoma 10 | 25.89720726 | 26.9535923 | −0.046 |
| BCL2 | B-cell CLL/lymphoma 2 | 30.98534203 | 31.99198151 | −0.013 |
| BCL2A1 | BCL2-related protein A1 | 33.95775223 | 34.95228577 | −0.005 |
| BCL2L1 | BCL2-like 1 | 29.06725121 | 31.03013992 | −0.401 |
| BCL2L10 | BCL2-like 10 (apoptosis facilitator) | 40 | 40 | 0.983 |
| BCL2L11 | BCL2-like 11 (apoptosis facilitator) | 26.96254349 | 27.96155167 | −0.008 |
| BCL2L13 | BCL2-like 13 (apoptosis facilitator) | 25.96006966 | 26.96482468 | −0.012 |
| BCL2L14 | BCL2-like 14 (apoptosis facilitator) | 33.96173859 | 33.9633255 | 0.981 |
| BCL2L2 | BCL2-like 2 | 31.99648666 | 31.98180008 | 1 |
| BCL3 | B-cell CLL/lymphoma 3 | 23.97451591 | 24.98425674 | −0.015 |
| BID | BH3 interacting domain death agonist | 23.97233772 | 24.9621315 | −0.001 |
| BIK | BCL2-interacting killer (apoptosis-inducing) | 26.97824669 | 26.96974564 | 0.995 |
| NAIP | NLR family, apoptosis inhibitory protein | 35.92926407 | 33.96829605 | 6.082 |
| BIRC3 | baculoviral IAP repeat-containing 3 | 28.96026993 | 30.95110893 | −0.501 |
| XLAP | X-linked inhibitor of apoptosis | 30.01272583 | 31.02407265 | −0.097 |
| BIRC5, | baculoviral IAP repeat-containing 5 | 22.98095703 | 23.97284889 | −0.085 |
| EPR1 | (survivin), effector cell peptidase receptor 1 | | | |
| BIRC6 | baculoviral IAP repeat-containing 6 (apollon) | 24.98253632 | 24.96587944 | 0.842 |
| BIRC7 | baculoviral IAP repeat-containing 7 (livin) | 36.98274231 | 36.99708557 | 0.802 |
| BIRC8 | baculoviral IAP repeat-containing 8 | 37.18276978 | 37.12633133 | 0.893 |
| BNIP3 | BCL2/adenovirus E1B 19 kDa interacting protein 3 | 24.96159172 | 24.91014862 | 1.055 |
| BNIP3L | BCL2/adenovirus E1B 19 kDa interacting protein | 24.94099426 | 25.96111679 | −0.102 |
| | 3-like | | | |
| BOK | BCL2-related ovarian killer | 27.98378372 | 29.98886299 | −0.546 |
| NOD2 | nucleotide-binding oligomerization domain | 32.00164795 | 32.9875412 | −0.081 |
| | containing 2 | | | |
| NOD1 | nucleotide-binding oligomerization domain | 29.96306801 | 30.9716835 | −0.095 |
| | containing 1 | | | |
| CARD6 | caspase recruitment domain family, member 6 | 31.93818092 | 32.94944382 | −0.097 |
| CARD9 | caspase recruitment domain family, member 9 | 35.96436691 | 35.98964691 | 0.949 |
| CASP1 | caspase 1, apoptosis-related cysteine peptidase | 35.93415833 | 35.96788788 | 0.937 |
| CASP10 | caspase 10, apoptosis-related cysteine peptidase | 31.9473629 | 32.94858932 | −0.091 |
| CASP14 | caspase 14, apoptosis-related cysteine peptidase | 27.96184731 | 28.94890976 | −0.082 |
| CASP2 | caspase 2, apoptosis-related cysteine peptidase | 24.95711708 | 25.94619751 | −0.083 |
| CASP3 | caspase 3, apoptosis-related cysteine peptidase | 40 | 40 | 0.983 |
| CASP4 | caspase 4, apoptosis-related cysteine peptidase | 27.96714211 | 27.95331383 | 1.002 |
| CASP5 | caspase 5, apoptosis-related cysteine peptidase | 35.89599991 | 36.94044113 | −0.117 |
| CASP6 | caspase 6, apoptosis-related cysteine peptidase | 25.9613266 | 25.95199394 | 0.996 |
| CASP7 | caspase 7, apoptosis-related cysteine peptidase | 24.9727211 | 25.96886063 | −0.087 |
| CASP8 | caspase 8, apoptosis-related cysteine peptidase | 27.94451714 | 27.9377212 | 0.993 |
| CASP8AP2 | CASP8 associated protein 2 | 26.92588806 | 27.94488144 | −0.102 |
| CASP9 | caspase 9, apoptosis-related cysteine peptidase | 25.93519974 | 26.94344521 | −0.095 |
| CFLAR | CASP8 and FADD-like apoptosis regulator | 27.97614098 | 27.96595764 | 0.833 |
| CHUK | conserved helix-loop-helix ubiquitous kinase | 25.96746445 | 25.91207314 | 0.892 |
| CRADD | CASP2 and RIPK1 domain containing adaptor | 26.96650314 | 27.96067047 | −0.086 |
| | with death domain | | | |
| DAPK1 | death-associated protein kinase 1 | 40 | 36.99488831 | 14.922 |
| DEDD | death effector domain containing | 23.94953156 | 24.95420265 | −0.093 |
| DEDD2 | death effector domain containing 2 | 33.98667526 | 32.97684479 | 2.993 |
| DIABLO | diablo homolog (Drosophila) | 23.95850945 | 24.96835518 | −0.096 |
| IFT57 | intraflagellar transport 57 homolog | 23.91511154 | 24.94013786 | −0.105 |
| FADD | Fas (TNFRSF6)-associated via death domain | 31.96405983 | 32.9624939 | −0.088 |
| FAS | Fas (TNF receptor superfamily, member 6) | 26.92230988 | 25.96039391 | 2.863 |
| FASLG | Fas ligand (TNF superfamily, member 6) | 37.00829315 | 37.11711121 | 0.839 |
| HIP1 | huntingtin interacting protein 1 | 27.96487236 | 29.00070381 | −0.112 |
| HRK | harakiri, BCL2 interacting protein | 38 | 38.0672493 | 0.893 |
| HTRA2 | HtrA serine peptidase 2 | 26.97640038 | 26.97018623 | 0.992 |
| ICEBERG | ICEBERG caspase-1 inhibitor | 29.96068573 | 29.96435928 | 0.978 |
| IKBKB | inhibitor of kappa light polypeptide gene enhancer | 25.92133904 | 26.94499207 | −0.104 |
| | in B-cells, kinase beta | | | |
| IKBKE | inhibitor of kappa light polypeptide gene enhancer | 26.96494484 | 27.94412041 | −0.077 |
| | in B-cells, kinase epsilon | | | |
| IKBKG | inhibitor of kappa light polypeptide gene enhancer | 23.96603203 | 24.97546959 | −0.096 |
| | in B-cells, kinase gamma | | | |
| LRDD | leucine-rich repeats and death domain containing | 28.99137688 | 28.97518921 | 0.841 |
| LTA | lymphotoxin alpha (TNF superfamily, member 1) | 33.9615593 | 30.96093941 | 14.872 |
| LTB | lymphotoxin beta (TNF superfamily, member 3) | 35.01607132 | 35.97927094 | −0.066 |
| MCL1 | myeloid cell leukemia sequence 1(BCL2-related) | 23.96264458 | 23.97689819 | 0.802 |
| NLRP1 | NLR family, pyrin domain containing 1 | 37.00725174 | 37.01711655 | 0.808 |
| NFKB1 | nuclear factor of kappa light polypeptide gene | 24.96630859 | 25.99078941 | −0.105 |
| | enhancer in B-cells 1 (p105) | | | |
| NFKB2 | nuclear factor of kappa light polypeptide gene | 24.97195053 | 25.98311996 | −0.096 |
| | enhancer in B-cells 2 (p49/p100) | | | |
| NFKBIA | nuclear factor of kappa light polypeptide gene | 24.9489975 | 25.97408104 | −0.105 |
| | enhancer in B-cells inhibitor, alpha | | | |
| NFKBIB | nuclear factor of kappa light polypeptide gene | 26.96529579 | 27.9742012 | −0.095 |
| | enhancer in B-cells inhibitor, beta | | | |
| NFKBIE | nuclear factor of kappa light polypeptide gene | 30.98664474 | 31.97193909 | −0.028 |
| | enhancer in B-cells inhibitor, epsilon | | | |
| NFKBIZ | nuclear factor of kappa light polypeptide gene | 25.96448708 | 26.96246719 | −0.088 |
| | enhancer in B-cells inhibitor, zeta | | | |
| PEA15 | phosphoprotein enriched in astrocytes 15 | 23.96767426 | 23.92144394 | 0.879 |
| PMAIP1 | phorbol-12-myristate-13-acetate-induced protein 1 | 27.97085953 | 28.9851265 | −0.099 |
| PYCARD | PYD and CARD domain containing | 27.97423744 | 29.97471428 | −0.545 |
| REL | v-relreticuloendotheliosis viral oncogene homolog | 26.92234612 | 27.91149902 | −0.083 |
| RELA | v-relreticuloendotheliosis viral oncogene homolog | 24.96393967 | 25.97424889 | −0.096 |
| | A, nuclear factor of kappa light polypeptide gene | | | |
| | enhancer in B-cells 3, p65 (avian) | | | |
| RELB | v-relreticuloendotheliosis viral oncogene homolog | 31.99829483 | 33.98251343 | −0.499 |
| | B, nuclear factor of kappa light polypeptide gene | | | |
| | enhancer in B-cells 3 (avian) | | | |
| RIPK1 | receptor (TNFRSF)-interacting serine-threonine | 26.96415901 | 26.96576691 | 0.981 |
| | kinase 1 | | | |
| RIPK2 | receptor-interacting serine-threonine kinase 2 | 25.00660324 | 24.87814331 | 1.168 |
| TBK1 | TANK-binding kinase 1 | 23.96338844 | 24.95111275 | −0.082 |
| TNF | tumor necrosis factor (TNF superfamily, member | 28.9993515 | 29.9725399 | −0.073 |
| | 2) | | | |
| TNFRSF10A | tumor necrosis factor receptor superfamily, | 26.95544815 | 27.9758873 | −0.103 |
| | member 10a | | | |
| TNFRSF10B | tumor necrosis factor receptor superfamily, | 25.98163033 | 26.9697361 | −0.082 |
| | member 10b | | | |
| TNFRSF1A | tumor necrosis factor receptor superfamily, | 22.94370651 | 23.94876671 | −0.093 |
| | member 1A | | | |
| TNFRSF1B | tumor necrosis factor receptor superfamily, | 40 | 40 | 0.821 |
| | member 1B | | | |
| TNFRSF21 | tumor necrosis factor receptor superfamily, | 25.99085236 | 25.98568726 | 0.991 |
| | member 21 | | | |
| TNFRSF25 | tumor necrosis factor receptor superfamily, | 34.00587845 | 33.98643494 | 1.011 |
| | member 25 | | | |
| TNFSF10 | tumor necrosis factor (ligand) superfamily, | 28.93013954 | 30.95192909 | −0.552 |
| | member 10 | | | |
| TRADD | TNFRSF 1A-associated via death domain | 31.9900951 | 31.9974823 | 0.973 |

TABLE S2
Primers and Probes Used in the Study

| Gene name | Primer (sense) | Primer (anti-sense) |
|---|---|---|
| huMCPIP1 (qPCR) | CCCATCACAGACCAGCACAT (SEQ ID NO: 1) | CTCGTAGGCCAGCTTCACAA (SEQ ID NO: 2) |
| mMCPIP1 (qPCR) | CCCCCTGACGACCCTTTA G (SEQ ID NO: 3) | GGCAGTGGTTTCTTACGAAGGA(SEQ ID NO: 4) |
| huGAPDH (qPCR) | AGTGATGGCATGGACTGTGGTCAT(SEQ ID NO: 5) | CATGTTCGTCATGGGTGTGAACCA (SEQ ID NO: 6) |
| mGAPDH (qPCR) | ACCCGTTTGGCTCCACCCTT (SEQ ID NO: 7) | AATGGGGTGAGGCCGGTGCT (SEQ ID NO: 8) |
| hup21 (qPCR) | GGAGACTCTCAGGGTCGAAA (SEQ ID NO: 9) | GGATTAGGGCTTCCTCTTGG (SEQ ID NO: 10) |
| huCyclin D1 | CCCTCGGTGTCCTACTTCAAA (SEQ ID NO: 11) | CACCTCCTCCTCCTCCTCTTC (SEQ ID NO: 12) |
| (qPCR) | | |
| huCDK2 (qPCR) | CCAGGAGTTACTTCTATGCCTGA (SEQ ID NO: 13) | TTCATCCAGGGGAGGTACAAC (SEQ ID NO: 14) |
| huCDK4 (qPCR) | ATGGCTACCTCTCGATATGAGC (SEQ ID NO: 15) | CATTGGGGACTCTCACACTCT (SEQ ID NO: 16) |
| huCDK6 (qPCR) | CCAGATGGCTCTAACCTCAGT (SEQ ID NO: 17) | AACTTCCACGAAAAAGAGGCTT(SEQ ID NO: 18) |
| huBcl2L1 (qPCR) | GAGCTGGTGGTTGACTTTCTC (SEQ ID NO: 19) | TCCATCTCCGATTCAGTCCCT (SEQ ID NO: 20) |
| huBcl2A1 (qPCR) | TACAGGCTGGCTCAGGACTAT (SEQ ID NO: 21) | CGCAACATTTTGTAGCACTCTG (SEQ ID NO: 22) |
| huBIRC3 (qPCR) | TTTCCGTGGCTCTTATTCAAACT (SEQ ID NO: 23) | GCACAGTGGTAGGAACTTCTCAT (SEQ ID NO: 24) |
| huRELB (qPCR) | CCATTGAGCGGAAGATTCAACT (SEQ ID NO: 25) | CTGCTGGTCCCGATATGAGG (SEQ ID NO: 26) |
| huBcl3 (qPCR) | AACCTGCCTACACCCCTATAC (SEQ ID NO: 27) | CACCACAGCAATATGGAGAGG (SEQ ID NO: 28) |
| huDEDD2 (qPCR) | TGAAGGCAAAGTGACCTGTGA (SEQ ID NO: 29) | AGGCGTCCAGATAGGAGAGC (SEQ ID NO: 30) |
| huFAS (qPCR) | TCTGGTTCTTACGTCTGTTGC (SEQ ID NO: 31) | CTGTGCAGTCCCTAGCTTTCC (SEQ ID NO: 32) |
| huBAD (qPCR) | AGGGAGGGCTGACCCAGAT (SEQ ID NO: 33) | GGCGGAAAACCCAAAACTTC (SEQ ID NO: 34) |
| huRIPK2 (qPCR) | GCCCTTGGTGTAAATTACCTGC (SEQ ID NO: 35) | GGACATCATGCGCCACTTT (SEQ ID NO: 36) |
| huDAPK1 (qPCR) | GCAGGAAAACGTGGATGATT (SEQ ID NO: 37) | CATTTCTTCACAACCGCA AA (SEQ ID NO: 38) |
| Luciferase | ATTTATCGGAGTTGCAGTTGCGCC (SEQ ID NO: | ACAAACACTACGGTAGGCTGCGAA(SEQ ID NO: 40) |
| (qPCR) | 39) | |
| huBIRC3 | GCATACTGAGACCCTGCCTTT (SEQ ID NO: 41) | CTCACAGAATTCTTTATAAAAACACC (SEQ ID NO: |
| (RNA-ChIP) | | 42) |
| huBcl2L1 | CGGGCTCTCTGCTGTACATAT (SEQ ID NO: 43) | AGCATCAGGCCGTCCAATCT (SEQ ID NO: 44) |
| (RNA-ChIP) | | |
| huBcl2L1 | TTCTAGACCAGACACTGACCATCC | GGCCGGCCTTCACTGAGTAAACACAGT |
| (full-length | ACTCTA (SEQ ID NO: 45) | TTAT (SEQ ID NO: 46) |
| 3UTR) | | |
| huBcl2A1 | TTCTAGAGAAGTTTGAACCTAAAT | GGCCGGCCTATAGAGAAAAATACATAC |
| (full-length | CTGGCTGG (SEQ ID NO: 47) | AATTTATTCA (SEQ ID NO: 48) |
| 3UTR) | | |
| huBIRC3 | TTCTAGAGAAGAACCAAAACATCGTC | GGCCGGCCTAAGAGTAATTTTTGTTGC TTT |
| (full-length | (SEQ ID NO: 49) | (SEQ ID NO: 50) |
| 3UTR) | | |
| huRELB | TTCTAGATGCCAGAGGAGGGGCAC TGGGT | GGCCGGCCGAGGCTCAAAAACTCA |
| (full-length | (SEQ ID NO: 51) | TCTTTATT (SEQ ID NO: 52) |
| 3UTR) | | |
| huBcl3 | TTCTAGAGGGGGATGGGGGGGCAG ATCTT | GGCCGGCCTATGGTACAAAAATAGTTT |
| (full-length | (SEQ ID NO: 53) | ATTAC (SEQ ID NO: 54) |
| 3UTR) | | |
| huBcl2L1 | TTCTAGACCAGACACTGACCATCC | AAATACAGACACGCTTAACAAAAATAGTAT |
| (truncated | ACTCTA (SEQ ID NO: 55) | ATCA (SEQ ID NO: 56) |
| 3UTR) | | |
| huBIRC3 | TTCTAGAGAAGAACCAAAACATCGTC | CCTTCGATGTATAGGACACTGATCAAA |
| (truncated | (SEQ ID NO: 57) | (SEQ ID NO: 58) |
| 3UTR) | | |
| huBcl2L1 | ATGTGTGAGGAGACATTGGCTTGCAGTGC | |
| (3′UTR-mut1) | GCG (SEQ ID NO: 59) | |
| huBcl2L1 | ATGTGTGAGGAGTCAATGGCTTGCAGTGC | |
| (3′UTR-mut2) | GCG (SEQ ID NO: 60) | |
| huBIRC3 | TGTGCATATATTCGGAATGACATTTTAGG | |
| (3′UTR-mut1) | (SEQ ID NO: 61) | |
| huBIRC3 | TGTGCATATATTCGGAATTGAATTTTAGGG | |
| (3′UTR-mut2) | ACATGGTG (SEQ ID NO: 62) | |

huBIRC3 (WT-Probe): 5′-UGCAUAUAUGUUGAAUGACAUU-3′ (SEQ ID NO: 63)
huBIRC3 (mut-Probe: 5′-UGCAUAUAUUCGGAAUGACAUU-3′ (SEQ ID NO: 64)
huBcl2L1 (WT-Probe): 5′-UGAGGAGCUGCUGGCUUGC-3′ (SEQ ID NO: 65)
huBcl2L1 (mut-Probe): 5′-UGAGGAGACAUUGGCUUGC-3′ (SEQ ID NO: 66)

### Example 3—Discussion

The inventors and others have demonstrated that the RNA-binding protein (RBP) tristetraprolin plays an important role in suppressing tumor progression through control of mRNA stability of pro-tumor genes (Sanduja et al., 2012, Ross et al., 2012 and Qian et al., 2013). MCPIP1 is a conserved zinc finger RBP with more than 85% homology between human and mouse. MCPIP1 has been shown to play an important role in suppressing chronic inflammation by promoting mRNA decay of proinflammatory cytokines. Using five isogenic tumorigenic lines that originated from one spontaneous tumor in the BALB/cfC3H mouse, the inventors found the most tumorigenic and metastatic 4T1 cells expressed the lowest levels of MCPIP1 (FIGS. 1H-I). This inverse correlation between MCPIP1 expression and metastatic potential suggests that MCPIP1 is a tumor suppressor. Unlike the well-known tumor suppressor p53, MCPIP1 is a zinc finger protein mainly localized in cytoplasm (FIGS. 8B-C). This unique location makes MCPIP1 a second layer to prevent tumorigenesis and tumor progression in addition of the nuclear p53.

When the inventors tried to express MCPIP1 in breast cancer cells, they encountered a difficulty in establishing a standard stable cell line due to massive death of tumor cells after MCPIP1 expression (FIGS. 9B-C). The relationship between MCPIP1 and apoptosis was initially suggested in cardiomyocytes (Zhou et al., 2006). The inventors later found that MCPIP1 itself does not induce apoptosis in macrophages but increases the sensitivity of macrophages to stress signal (Qi et al., 2011). To the inventors' knowledge, so far, there is no direct evidence in inducing apoptosis by MCPIP1 in breast tumor cells. Using different methods, they demonstrate that MCPIP1 can strongly induce apoptosis in breast cancer cells (FIGS. 2A-F). Apoptosis is triggered upon the balance between pro-apoptotic and anti-apoptotic gene being broken. MCPIP1 inhibited the expression of anti-apoptotic gene transcripts (FIGS. 3B-E) while has minor or no effects on pro-apoptotic gene transcripts (FIGS. 3F-I). The binding of MCPIP1 to anti-apoptotic mRNAs but not to pro-apoptotic mRNAs further confirms a select targeting of MCPIP1 in apoptosis pathway, causing imbalance between pro-apoptotic and anti-apoptotic genes. The inventors have also observed that MCPIP1 induce apoptosis of other types of cells, including HEK293 cells, Jurkat cells and CEM cells (data not shown), indicating that MCPIP1 may play a broader role in regulation of apoptosis pathway. Collectively, these results indicate that MCPIP1 suppresses breast tumor progression through induction of cell apoptosis.

Steady-state mRNA level is determined by both transcription and mRNA stability. These results indicate that MCPIP1 suppresses the expression of anti-apoptotic genes at the post-transcriptional level through enhancing mRNA degradation. Among the MCPIP1-targeting genes, Bcl2L1 (also called Bcl-xl) (Eichhom et al., 2013) and Bcl2A1 (Ottina et al., 2012) are mainly involved in intrinsic mitochondria anti-apoptosis signaling pathway, while BIRC3 (Zamegar et al., 2008), RelB (Tando et al., 2010), and Bcl3 (Maldonado and Melendez-Zajgla, 2011) are involved in activation of NF-kB pathway. The broad targets of MCPIP1 in apoptosis pathway suggest that MCPIP1 induces apoptosis through multi molecular events in breast tumor cells. It has been known that the elements regulating mRNA decay are mainly located in the 3′UTR, such as ARE and GRE (Vlasova-St Louis and Bohjanen, 2014 and Ivanov and Anderson, 2013). Indeed, MCPIP1 inhibits luciferase activity through the 3′UTRs of anti-apoptotic genes (FIG. 4E). In contrast to TTP (Sandler et al., 2011) and Roquin (Leppek et al., 2013) which recruit other decadenylases to the 3′UTR for mRNA degradation, MCPIP1 is known to decay its target mRNAs by itself. It has been reported that MCPIP1 possesses a PIN-like domain at its N-terminal with endonuclease activity (Xu et al., 2012). This PIN-domain is essential for MCPIP1's mRNA degradation and anti-viral effects. By mutating PIN and ZF domain, the inventors demonstrate that the PIN-domain is also responsible for apoptosis induction. MCPIP1 destabilizes mRNA stability of IL-6 and IL-2 by targeting stem-loop but ARE in the 3′UTR. These results demonstrate that it is the stem-loop structure but not the absolute nucleotide sequence required for MCPIP1-mediated mRNA decay. Roquin has been shown to recognize a constitutive decay element (CDE) in the 3′UTR of TNF-α mRNAs and this CDE can fold into a stem-loop structure (Leppek et al., 2013). The inventors found no consensus stem-loop sequences among the five anti-apoptotic genes, which support their hypothesis that MCPIP1 mainly recognizes the secondary hairpin structure but not linear sequence in the 3′UTR. MCPIP1 could cleave and process pre-miRNAs via the specific loop structure (Suzuki et al., 2011). These results demonstrate that MCPIP1 seems to modulate not only biogenesis of the miRNAs, but also translation of the mRNAs important for tumor growth and apoptosis. As a RBP localized in cytoplasm, MCPIP1 readily controls the fate of tumor cells by tipping the balance towards apoptosis.

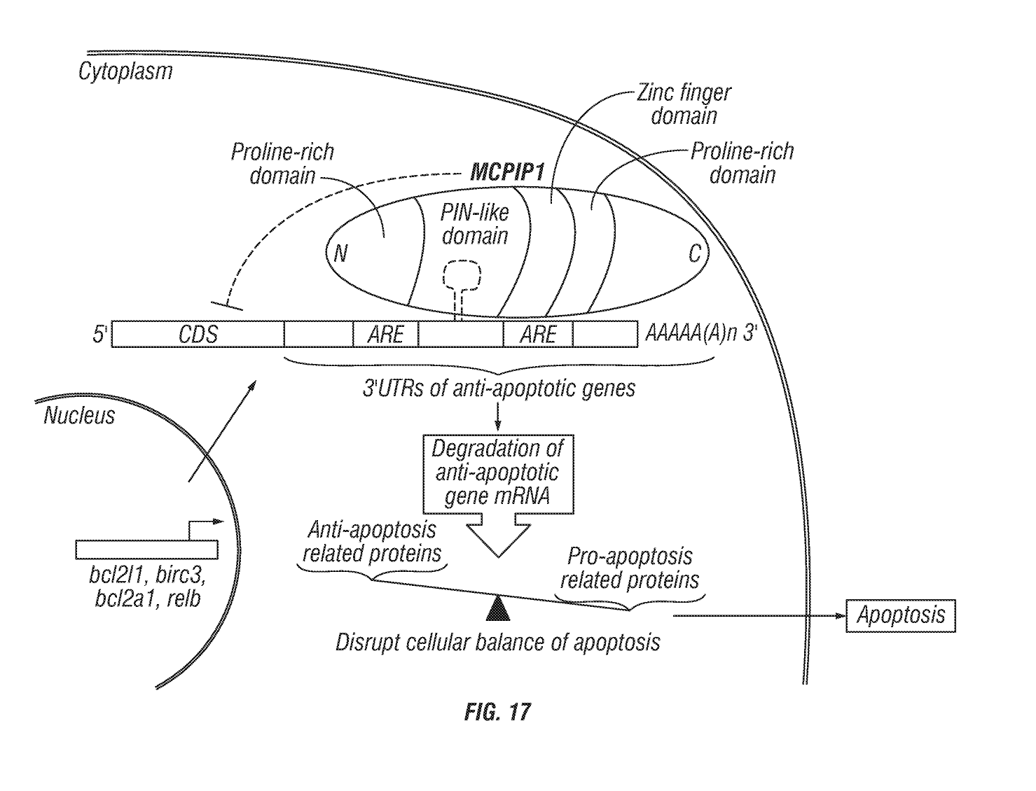

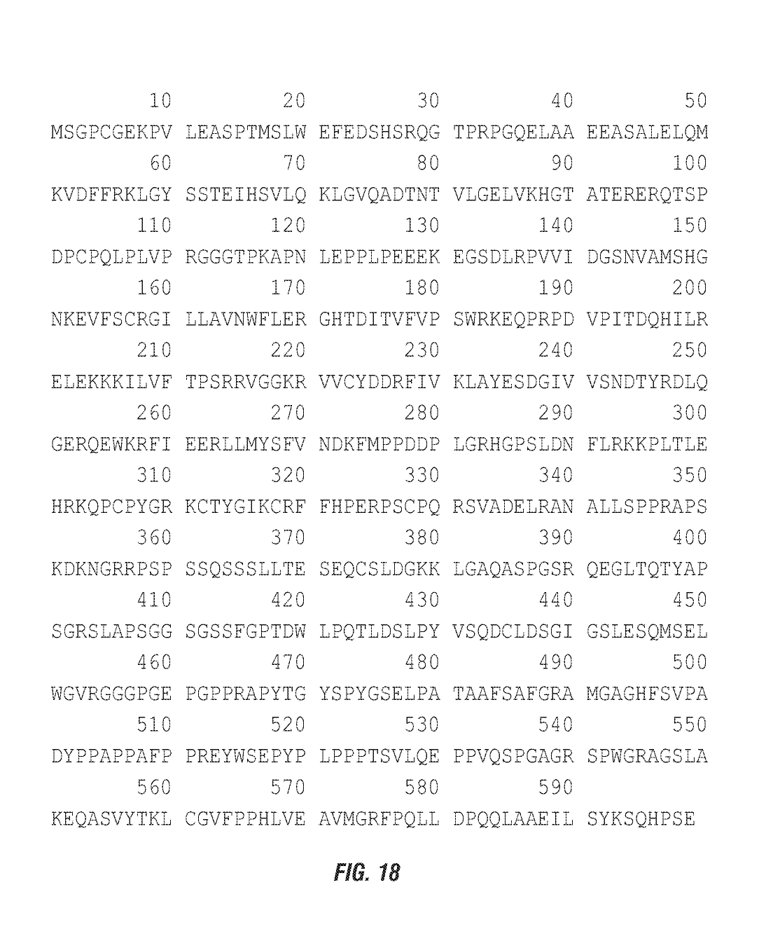

Based on this study, the inventors propose a model to illustrate the potent role for MCPIP1 in suppression of breast tumor growth and metastasis (FIG. 17). Though the primary tumors are completely eliminated in mice fed with Dox water, there are still a few metastatic nodules and cells presented in the lungs. To find out the reason, the inventors isolated the metastatic tumor cells from lung and cultured them in vitro. They found that the remained lung metastatic tumor cells can still express MCPIP1 after adding Dox into culture medium (FIG. 15D) and die within a few days (data not shown), indicating that MCPIP1 was not induced in those cells previously. The possible reason for it may be due to a low concentration of Dox in the lung, which fails to reach sufficient level for inducing MCPIP1 in lung tumor cells. It also suggests that increasing the concentration of Dox in metastatic organs, including lung, should be able to eliminate metastatic cells entirely. Therefore, drugs or small molecules able to induce MCPIP1 expression can be used therapeutically for treatment of metastatic breast cancer. The strong association between levels of MCPIP1 in tumors and survival of patients with breast tumor over 13 years follow-up further demonstrate the biological relevance of inducing MCPIP1 for breast tumor treatment.

Taken together, these results demonstrate that MCPIP1 is a potent tumor suppressor by inducing tumor apoptosis. MCPIP1 selectively targets the mRNAs of anti-apoptotic genes for degradation through the PIN domain via recognizing and binding to a stem-loop structure in the 3′UTRs. As an RNA-binding protein localized in the cytoplasm, MCPIP1 can quickly control cell fate by tipping the balance towards apoptosis in tumor cells. These findings also add a new regulator in the apoptosis pathway for apoptosis.

All of the compositions and methods disclosed and claimed herein can be made and executed without undue experimentation in light of the present disclosure. While the compositions and methods of this disclosure have been described in terms of particular embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference:
U.S. Pat. No. 4,554,101
U.S. Pat. No. 4,683,195
U.S. Pat. No. 4,683,202
U.S. Pat. No. 4,684,611
U.S. Pat. No. 4,879,236
U.S. Pat. No. 4,952,500
U.S. Pat. No. 5,217,879
U.S. Pat. No. 5,302,523
U.S. Pat. No. 5,322,783
U.S. Pat. No. 5,384,253
U.S. Pat. No. 5,464,765
U.S. Pat. No. 5,506,138
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,656,610
U.S. Pat. No. 5,670,488
U.S. Pat. No. 5,702,932
U.S. Pat. No. 5,736,524
U.S. Pat. No. 5,739,018
U.S. Pat. No. 5,780,448
U.S. Pat. No. 5,789,215
U.S. Pat. No. 5,824,544
U.S. Pat. No. 5,830,725
U.S. Pat. No. 5,849,304
U.S. Pat. No. 5,851,826
U.S. Pat. No. 5,858,744
U.S. Pat. No. 5,871,982
U.S. Pat. No. 5,871,983
U.S. Pat. No. 5,871,986
U.S. Pat. No. 5,879,934
U.S. Pat. No. 5,888,502
U.S. Pat. No. 5,925,565
U.S. Pat. No. 5,928,906
U.S. Pat. No. 5,932,210
U.S. Pat. No. 5,945,100
U.S. Pat. No. 5,955,331
U.S. Pat. No. 5,981,274
U.S. Pat. No. 5,994,136
U.S. Pat. No. 5,994,624
U.S. Pat. No. 6,013,516
EPO 0273085
Almendro et al., J. Immunol., 157(12):5411-5421, 1996.
Amado and Chen, Science, 285(5428):674-676, 1999.
Armentano et al., Proc. Natl. Acad. Sci. USA, 87(16):6141-6145, 1990.
Ausubel et al., In: Current Protocols in Molecular Biology, John, Wiley & Sons, Inc, New York, 1994.
Batra et al., Am. J. Respir. Cell Mol. Biol., 21(2):238-245, 1999.
Bellus, J. Macromol. Sci. Pure Appl. Chem., A31(1): 1355-1376, 1994.
Bett et al., J. Virololgy, 67(10):5911-5921, 1993.
Bilbao et al., Transplant Proc., 31(1-2):792-793, 1999.
Blackwell et al., Arch. Otolaryngol. Head. Neck Surg., 125(8):856-863, 1999.
Blomer et al., J. Virol., 71(9):6641-6649, 1997.
Capaldi et al., Biochem. Biophys. Res. Comm., 76:425, 1977.
Caplen et al., Gene Ther., 6(3):454-459, 1999.
Case et al., Proc. Natl. Acad. Sci. USA, 96(6):2988-2893, 1999.
Chandler et al., Proc. Natl. Acad. Sci. USA, 94(8):3596-601, 1997.
Chen and Okayama, Mol. Cell. Biol. 7:2745-2752, 1987.
Chillon et al., J. Virol., 73(3):2537-2540, 1999.
Clay et al., Pathol. Oncol. Res., 5(1):3-15, 1999.
Coffey et al., Science, 282(5392): 1332-1334, 1999.
Culver et al., Science, 256(5063): 1550-1552, 1992.
DeLuca et al., J. Virol., 56(2):558-570, 1985.
Derby et al., Hear Res., 134(1-2): 1-8, 1999.
Dorai et al., Int. J. Cancer, 82(6):846-852, 1999.
Eichhom et al., Cell Death Dis, 4: p. e834, 2013.
Engel and Kohn, Front Biosci., 4:e26-33, 1999.
Fechheimer, et al., Proc Natl. Acad. Sci. USA, 84:8463-8467, 1987.
Feldman et al., Semin. Interv. Cardiol., 1(3):203-208, 1996.
Feng et al., Nat. Biotechnol., 15(9):866-870, 1997.
Fisher et al., Virology, 217(1): 11-22, 1996.
Fraley et al., Proc. Natl. Acad. Sci. USA, 76:3348-3352, 1979.
Fujiwara and Tanaka, Nippon Geka Gakkai Zasshi, 99(7):463-468, 1998.
Garoffand Li, Curr. Opin. Biotechnol., 9(5):464-469, 1998.
Garrido et al., J. Neurovirol., 5(3):280-288, 1999.
Ghosh and Bachhawat, In: Liver Diseases, Targeted Diagnosis and Therapy Using Specific Receptors and Ligands, Wu and Wu (Eds.), Marcel Dekker, New York, 87-104, 1991.
Gnant et al., Cancer Res., 59(14):3396-403, 1999.
Gnant et al., J. Natl. Cancer Inst., 91(20): 1744-1750, 1999.
Gopal, Mol. Cell. Biol., 5:1188-1190, 1985.
Graham and Prevec, Mol. Biotechnol., 3(3):207-220, 1995.
Graham and Van Der Eb, Virology 52:456-467, 1973
Haecker et al., Hum. Gene Ther., 7(15): 1907-1914, 1996.
Han et al., Euro. J. Surgical Oncology, 25:194-198, 1999.
Hanahan and Weinberg, Cell, 144(5): p. 646-74, 2011.
Harland and Weintraub, J. Cell Biol., 101:1094-1099, 1985.
Hayashi et al., Neurosci. Lett., 267(1):37-40, 1999.
He et al., Arterioscler Thromb Vasc Biol, 33(6): p. 1384-91, 2013.
Hermens and Verhaagen, Prog. Neurobiol., 55(4):399-432, 1998.
Hofacker, Nucleic Acids Res, 31(13): p. 3429-31, 2003.
Hollmann et al., Oncogene, 20(52): p. 7645-57, 2001.
Holzer et al., Virology, 253(1):107-114, 1999.
Howard et al., Ann. NY Acad. Sci., 880:352-365, 1999.
Huang et al., Cell Signal, 25(5): p. 1228-34, 2013.
Huard et al., Neuromuscul Disord., 7(5):299-313, 1997.
Igney and Krammer, Nat Rev Cancer, 2(4): p. 277-88, 2002.
Imai et al., J. Virol., 72(5):4371-4378, 1998.
Irie et al., Antisense Nucleic Acid Drug Dev., 9(4):341-349, 1999.
Ivanov and Anderson, Immunol Rev, 253(1): p. 253-72, 2013.
Iwasaki et al., Nat Immunol, 12(12): p. 1167-75, 2011.
Johnston et al., J. Virol., 73(6):4991-5000, 1999.
Kaeppler et al., Plant Cell Reports, 9:415-418, 1990.
Kaneda et al., Science, 243:375-378, 1989.
Kang and Reynolds, Clin Cancer Res, 15(4): p. 1126-32, 2009.
Kato et al., J. Biol. Chem., 266(6):3361-3364, 1991.
Kaufman et al., Surv. Ophthalmol., 43 Suppl 1:S91-97, 1999.
Kay, Haemophilia, 4(4):389-392, 1998.
Keene et al., Nat Protoc, 1(1): p. 302-7, 2006.
Klimatcheva et al., Front Biosci., 4:D481-96, 1999.
Kohut et al., Am. J. Physiol., 275(6 Pt 1):L1089-94, 1998.
Kooby et al., FASEB J., 13(11):1325-1334, 1999.
Kraus et al., FEBS Lett., 428(3):165-170, 1998.
Krisky et al., Gene Ther., 5(11):1517-1530, 1998a
Krisky et al., Gene Ther., 5(12):1593-1603, 1998b.
Kumar et al., Endocr Relat Cancer, 7(4): p. 257-69, 2000.
Lachmann and Efstathiou, Clin. Sci. (Colch), 96(6):533-541, 1999.
Lareyre et al., J. Biol. Chem., 274(12):8282-8290, 1999.
Leibowitz et al., Diabetes, 48(4):745-753, 1999.
Leppek et al., Cell, 153(4): p. 869-81, 2013.
Lesch, Biol. Psychiatry, 45(3):247-253, 1999.
Li et al., PLoS One, 7(11): p. e49841, 2012.
Liang et al., J Biol Chem, 283(10): p. 6337-46, 2008.
Liang et al., J Exp Med, 207(13): p. 2959-73, 2010.
Lin et al., J Immunol, 193(8): p. 4159-68, 2014.
Lin et al., Nucleic Acids Res, 41(5): p. 3314-26, 2013.
Liu et al., Proc Natl Acad Sci USA, 110(47): p. 19083-8, 2013.
Lowe and Lin, Carcinogenesis, 21(3): p. 485-95, 2000.
Lu et al., J Biol Chem, 285(13): p. 9317-21, 2010.
Lundstrom, J. Recept. Signal Transduct. Res., 19(1-4):673-686, 1999.
Lyu et al., J Cell Biochem, 2014.
Maldonado and Melendez-Zajgla, Mol Cancer, 10: p. 152, 2011.
Marienfeld et al., Gene Ther., 6(6): 1101-1113, 1999.
Mastrangelo et al., Cancer Gene Ther., 6(5):409-422 1999.
Matsushita et al., Nature, 458(7242): p. 1185-90, 2009.
Miller et al., Methods Enzymol., 217:581-599, 1993.
Miller et al., Proc Natl Acad Sci USA, 102(38): p. 13550-5, 2005.
Miyatake et al., Gene Ther., 6(4):564-572, 1999.
Moldawer et al., Shock, 12(2):83-101, 1999.
Moriuchi et al., Cancer Res., 58(24):5731-5737, 1998.
Morrison et al., J. Gen. Virol., 78(Pt 4):873-878, 1997.
Naldini et al., Proc. Natl. Acad. Sci. USA, 93(21): 11382-11388, 1996.
Nicolau and Sene, Biochim. Biophys. Acta, 721:185-190, 1982.
Nicolau et al., Methods Enzymol., 149:157-176, 1987
Nomoto et al., Gene, 236(2):259-271, 1999.
Omirulleh et al., Plant Mol. Biol., 21(3):415-28, 1993.
Ottina et al., Blood, 119(25): p. 6032-42, 2012.
Parks et al., J. Virol., 71(4):3293-8, 1997.
Perales et al., Proc. Natl. Acad. Sci. USA, 91:4086-4090, 1994.
Petrof, Eur. Respir. J., 11(2):492-497, 1998.
Placzek et al., Cell Death Dis, 1: p. e40, 2010.
Potrykus et al., Mol. Gen. Genet., 199:183-188, 1985.
Potter et al., Proc. Natl. Acad. Sci. USA, 81:7161-7165, 1984.
Qi et al., FEBS Lett, 584(14): p. 3065-72, 2010.
Qi et al., J Biol Chem, 286(48): p. 41692-700, 2011.
Qian et al., J Immunol, 186(11): p. 6454-64, 2011.
Qian et al., J Immunol, 190(11): p. 5894-902, 2013.
Rabinovitch et al., Diabetes, 48(6): 1223-1229, 1999.
Reddy et al., J. Virol., 72(2):1394-1402, 1998.
Remington's Pharmaceutical Sciences, 15^{th} ed., pages 1035-1038 and 1570-1580, Mack Publishing Company, Easton, Pa., 1980.
Remington's Pharmaceutical Sciences 15th Edition, 33:624-652, 1990
Rippe et al., “DNA-mediated gene transfer into adult rat hepatocytes in primary culture,” Mol. Cell Biol., 10:689-695, 1990.
Robbins and Ghivizzani, Pharmacol. Ther., 80(1):35-47, 1998.
Robbins et al., Proc. Natl. Acad. Sci. USA, 95(17):10182-10187 1998.
Robbins et al., Trends Biotechnol., 16(1):35-40, 1998.
Ross et al., Ageing Res Rev, 11(4): p. 473-84, 2012.
Sambrook et al., In:Molecular Cloning: A Laboratory Manual, Vol. 1, Cold Spring Harbor Laboratory Press, Cold Spring Harbor, N.Y., Ch. 7, 7.19-17.29, 1989.
Sandler et al., Nucleic Acids Res, 39(10): p. 4373-86, 2011.
Sanduja et al., Front Biosci (Landmark Ed), 17: p. 174-88, 2012.
Sawai et al., Mol. Genet. Metab., 67(1):36-42, 1999.
Skalniak et al., FEBS J, 280(11): p. 2665-74, 2013.
Skalniak et al., Oncol Rep. 31(5): p. 2385-92, 2014.
Smith, Arch. Neurol., 55(8):1061-1064, 1998.
Steller, Science, 267(5203): p. 1445-9, 1995.
Stewart et al., Gene Ther., 6(3):350-363, 1999.
Suzuki et al., Biochem. Biophys. Res. Commun., 252(3):686-690, 1998.
Suzuki et al., Mol Cell, 44(3): p. 424-36, 2011.
Tanaka et al., Oncogene, 8:2253-2258, 1993.
Tando et al., J Biol Chem, 285(29): p. 21951-60, 2010.
Thompson, Science, 267(5203): p. 1456-62, 1995.
Timiryasova et al., Int. J. Oncol., 14(5):845-854, 1999.
Timiryasova et al., Oncol. Res.; 11(3): 133-144, 1999.
Tsumaki et al., J. Biol. Chem., 273(36):22861-22864, 1998.
Tur-Kaspa et al., Mol. Cell Biol., 6:716-718, 1986.
Uehata et al., Cell, 153(5): p. 1036-49, 2013.
Vanderkwaak et al., Gynecol. Oncol., 74(2):227-234, 1999.
Vlasova-St Louis and Bohjanen, J Interferon Cytokine Res, 34(4): p. 233-41, 2014.
Wagner et al., Science, 260:1510-1513, 1990.
Wang et al., Gynecol. Oncol., 71(2):278-287, 1998.
Weihl et al., Neurosurgery, 44(2):239-252, 1999.
White et al., J. Virol., 73(4):2832-28340, 1999.
Wilson, J. Clin. Invest., 98(11):2435, 1996.
Wong et al., Gene, 10:87-94, 1980.
Wu and Wu, J. Biol. Chem., 262:4429-4432, 1987.
Wu et al., Biochem. Biophys. Res. Commun., 233(1):221-226, 1997.
Wu, Chung Hua Min Kuo Hsiao Erh Ko I Hsueh Hui Tsa Chih, 39(5):297-300, 1998.
Xu et al., Nucleic Acids Res, 40(14): p. 6957-65, 2012.
Yamada et al., Proc. Natl. Acad. Sci. USA, 96(7):4078-4083, 1999.
Yeung et al., Gene Ther., 6(9):1536-1544, 1999.
Yip and J. C. Reed, Oncogene, 27(50): p. 6398-406, 2008.
Yoon et al., J. Gastrointest. Surg., 3(1):34-48, 1999.
Zarnegar et al., Nat Immunol, 9(12): p. 1371-8, 2008.
Zhao-Emonet et al., Biochim. Biophys. Acta, 1442(2-3): 109-119, 1998.
Zheng et al., J. Gen. Virol., 80(Pt 7):1735-1742, 1999.
Zhou et al., Circ Res, 98(9): p. 1177-85, 2006.
Zufferey et al., Nat. Biotechnol., 15(9):871-875, 1997.

1. An isolated polynucleotide encoding MCPIP1, wherein said isolated polynucleotide is operably connected to a heterologous promoter active in a mammalian cancer cell.

2. The polynucleotide of claim 1, wherein.

3. The polynucleotide of claim 1, wherein said heterologous promoter is operable in human cancer cell.

4. The polynucleotide of claim 1, wherein said heterologous promoter is active in a non-human mammal cancer cell.

5. The polynucleotide of claim 1, wherein said promoter is selected from the group consisting of hsp68, SV40, CMV IE, MKC, GAL4_{UAS}, HSV and β-actin.

6. The polynucleotide of claim 1, wherein said promoter is a tissue specific promoter.

7. The polynucleotide of claim 1, wherein said promoter is an inducible promoter.

8. The polynucleotide of claim 1, wherein said promoter is active in a human breast cancer cell or a human melanoma cell.

9. The polynucleotide of claim 1, wherein said polynucleotide is contained in a replicable expression vector.

10. (canceled)

11. A method for suppressing the growth, proliferation, migration or metastasis of a cancer cell comprising contacting said cells with an expression cassette comprising a polynucleotide encoding MCPIP1, wherein said polynucleotide is under the control of a promoter operable in eukaryotic cells.

12. The method of claim 11, wherein said promoter is heterologous to the polynucleotide sequence.

13. The method of claim 12, wherein said promoter is selected from the group consisting of hsp68, SV40, CMV, MKC, GAL4_{UAS}, HSV and β-actin.

14. The method of claim 11, wherein said promoter is a tissue specific promoter.

15. The method of claim 11, wherein said promoter is an inducible promoter.

16. The method of claim 11, wherein said expression cassette is contained in a replicable expression vector.

17-18. (canceled)

19. The method of claim 11, wherein said expression cassette further comprises a polyadenylation signal.

20-21. (canceled)

22. The method of claim 11, further comprising contacting said cancer cell with a second anti-cancer therapy.

23. (canceled)

24. The method of claim 11, wherein said cancer cell is derived from a tissue selected from the group consisting of brain, lung, liver, spleen, kidney, lymph node, small intestine, blood, pancreas, colon, stomach, breast, endometrium, prostate, testicle, ovary, skin, head and neck, esophagus, bone marrow and blood tissue.

25. The method of claim 11, wherein said cancer cell is located in a human subject.

26-28. (canceled)

29. The method of claim 11, wherein said expression cassette is administered more than once.

30. The method of claim 22, further comprising assessing MCPIP1 structure, expression and/or function in a sample from said subject.

31. A method for suppressing the growth, proliferation, migration or metastasis of a cancer cell comprising the step of contacting a cancer cell with MCPIP1 polypeptide.

32-41. (canceled)
